SEQUENCE LISTING (75 sequences)

SEQ ID NO: 1 - protein, 1368 aa, Streptococcus pyogenes [sequence omitted]

SEQ ID NO: 2 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 8 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 9 - protein, 1306 aa, unidentified
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 10 - protein, 1306 aa, unidentified
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMANSNAATGEDYINSPVRDLNGVCFDSRFQNPEWPMDADANGAYHIALKGQLLLNHLKESKDLKLQNGISNQDWLAYIQELRN

SEQ ID NO: 11 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 12 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 13 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 14 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 15 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 16 - protein, 1285 aa, Herbinix hemicellulosilytica
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 17 - DNA, 18 bp, synthetic construct
  source 1..18: mol_type = other DNA
taatacgactcactatag

SEQ ID NO: 18 - DNA, 18 bp, synthetic construct
  source 1..18: mol_type = other DNA
atttaggtgacactatag

SEQ ID NO: 19 - protein, 9 aa, synthetic construct
LPPLERLTL

SEQ ID NO: 20 - protein, 10 aa, synthetic construct
LALKLAGLDL

SEQ ID NO: 21 - protein, 11 aa, synthetic construct
LSQALASSFSV

SEQ ID NO: 22 - protein, 12 aa, synthetic construct
NELALKLAGLDI

SEQ ID NO: 23 - protein, 9 aa, synthetic construct
LPPLERLTL

SEQ ID NO: 24 - protein, 7 aa, synthetic construct
PKKKRKV

SEQ ID NO: 25 - protein, 16 aa, synthetic construct
KRPAATKKAGQAKKKK

SEQ ID NO: 26 - protein, 9 aa, synthetic construct
PAAKRVKLD

SEQ ID NO: 27 - protein, 11 aa, synthetic construct
RQRRNELKRSP

SEQ ID NO: 28 - protein, 38 aa, synthetic construct
NQSSNFGPMKGGNFGGRSSGPYGGGGQYFAKPRNQGGY

SEQ ID NO: 29 - protein, 42 aa, synthetic construct
RMRIZFKNKGKDTAELRRRRVEVSVELRKAKKDEQILKRRNV

SEQ ID NO: 30 - protein, 8 aa, synthetic construct
VSRKRPRP

SEQ ID NO: 31 - protein, 8 aa, synthetic construct
PPKKARED

SEQ ID NO: 32 - protein, 8 aa, synthetic construct
PQPKKKPL

SEQ ID NO: 33 - protein, 12 aa, synthetic construct
SALIKKKKKMAP

SEQ ID NO: 34 - protein, 5 aa, synthetic construct
DRLRR

SEQ ID NO: 35 - protein, 7 aa, synthetic construct
PKQKKRK

SEQ ID NO: 36 - protein, 10 aa, synthetic construct
RKLKKKIKKL

SEQ ID NO: 37 - protein, 10 aa, synthetic construct
REKKKFLKRR

SEQ ID NO: 38 - protein, 20 aa, synthetic construct
KRKGDEVDGVDEVAKKKSKK

SEQ ID NO: 39 - protein, 17 aa, synthetic construct
RKCLQAGMNLEARKTKK

SEQ ID NO: 40 - protein, 30 aa, synthetic construct
MDSLLMNRRKFLYQFKNVRWAKGRRETYLC

SEQ ID NO: 41 - protein, 20 aa, synthetic construct
GLFXALLXLLXSLWXLLLXA

SEQ ID NO: 42 - protein, 20 aa, synthetic construct
GLFHALLHLLHSLWHLLLHA

SEQ ID NO: 43 - protein, 167 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 44 - protein, 178 aa, synthetic construct
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 45 - protein, 160 aa, synthetic construct
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 46 - protein, 161 aa, synthetic construct
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 47 - protein, 183 aa, synthetic construct
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 48 - protein, 164 aa, synthetic construct
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 49 - protein, 173 aa, synthetic construct
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 50 - protein, 161 aa, synthetic construct
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 51 - protein, 179 aa, synthetic construct
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 52 - protein, 198 aa, synthetic construct [sequence omitted]

SEQ ID NO: 53 - protein, 188 aa, synthetic construct
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 54 - protein, 198 aa, synthetic construct [sequence omitted]

SEQ ID NO: 55 - protein, 11 aa, synthetic construct
YGRKKRRQRRR

SEQ ID NO: 56 - protein, 12 aa, synthetic construct
RRQRRTSKLMKR

SEQ ID NO: 57 - protein, 27 aa, synthetic construct
GWTLNSAGYLLGKINLKALAALAKKIL

SEQ ID NO: 58 - protein, 33 aa, synthetic construct
KALAWEAKLAKALAKALAKHLAKALAKALKCEA

SEQ ID NO: 59 - protein, 16 aa, synthetic construct
RQIKIWFQNRRMKWKK

SEQ ID NO: 60 - protein, 9 aa, synthetic construct
RKKRRQRRR

SEQ ID NO: 61 - protein, 8 aa, synthetic construct
RKKRRQRR

SEQ ID NO: 62 - protein, 11 aa, synthetic construct
YARAAARQARA

SEQ ID NO: 63 - protein, 11 aa, synthetic construct
THRLPRRRRRR

SEQ ID NO: 64 - protein, 11 aa, synthetic construct
GGRRARRRRRR

SEQ ID NO: 65 - protein, 5 aa, synthetic construct
GSGGS

SEQ ID NO: 66 - protein, 6 aa, synthetic construct
GGSGGS

SEQ ID NO: 67 - protein, 5 aa, synthetic construct
GGGGS

SEQ ID NO: 68 - protein, 4 aa, synthetic construct
GGGS

SEQ ID NO: 69 - protein, 4 aa, synthetic construct
GGSG

SEQ ID NO: 70 - protein, 5 aa, synthetic construct
GGSGG

SEQ ID NO: 71 - protein, 5 aa, synthetic construct
GSGSG

SEQ ID NO: 72 - protein, 5 aa, synthetic construct
GSGGG

SEQ ID NO: 73 - protein, 5 aa, synthetic construct
GGGSG

SEQ ID NO: 74 - protein, 5 aa, synthetic construct
GSSSG

SEQ ID NO: 75 - protein, 5 aa, synthetic construct
GGGGS

(19) United States
(12) Patent Application Publication
(10) Pub. No.: US 2026/0357249 A1
(43) Pub. Date: Nov. 12, 2026

(54) CRISPR-Cas Guide Nucleic Acids and Methods of Use Thereof

(71) Applicant: The Board of Trustees of the Leland Stanford Junior University, Stanford, CA (US)

(72) Inventors: Lei S. Qi, Stanford, CA (US); Yanyu Zhu, Redwood City, CA (US)

(21) Appl. No.: 19/546,081

(22) Filed: Feb. 20, 2026

Related U.S. Application Data
(60) Provisional application No. 63/762,301, filed on Feb. 24, 2025.

(51) Int. Cl. C12N 15/11; C12N 9/22; C12N 15/90

(52) U.S. Cl. CPC C12N 15/11; C12N 9/226; C12N 15/907; C12N 2310/20

(57) ABSTRACT

The present disclosure provides methods for generating a list of chromatin accessible target sequences for CRISPR-Cas guide RNAs that target a contiguous genomic region. The present disclosure provides pluralities of in vitro transcribed CRISPR-Cas guide RNAs that target a contiguous genomic region and template nucleic acids encoding single the guide RNAs (e.g., single guide RNAs). The present disclosure additionally provides a CRISPR-Cas guide RNA comprising: a guide sequence that hybridizes to a target sequence; a scaffold sequence that can bind to a CRISPR-Cas effector protein; and a 3′ poly(A) tail, wherein at least one adenosine of the poly(A) tail comprises a heterologous moiety. Lastly, the present disclosure provides methods for preparing a plurality of in vitro transcribed CRISPR-Cas guide RNAs, methods for labeling CRISPR-Cas guide RNAs, and methods for guiding a plurality of CRISPR-Cas effector complexes to a contiguous genomic region of interest.

### CROSS-REFERENCE

This application claims the benefit of U.S. Provisional Patent Application No. 63/762,301 filed Feb. 24, 2025, which application is incorporated herein by reference in its entirety.

### STATEMENT REGARDING FEDERALLY SPONSORED RESEARCH

This invention was made with Government support under contract 2046650 awarded by the National Science Foundation and under contracts DK127405, HG013133, and NS137219 awarded by the National Institutes of Health. The Government has certain rights in the invention.

### INCORPORATION BY REFERENCE OF SEQUENCE LISTING PROVIDED AS AN XML FILE

A Sequence Listing is provided herewith as a Sequence Listing XML, “STAN-2249_SeqList.xml” created on Feb. 18, 2026 and having a size of 88,550 bytes. The contents of the Sequence Listing XML are incorporated by reference herein in their entirety.

### INTRODUCTION

The three-dimensional (3D) organization and dynamic communication of the eukaryotic genome play crucial roles in regulating gene transcription and cellular functions. Aberrations in the 3D genome architecture are linked to many diseases, including cancers, neurological disorders, and developmental abnormalities. However, key questions such as the dynamics of 3D genome communication, the functional relationship between the 3D structure and gene expression, and how alterations in these interactions contribute to disease remain unclear, due to the lack of robust approaches for labeling and tracking non-repetitive genomic regions in live cells in real time.

Recent imaging and sequencing-based methods, such as multiplexed fluorescence in situ hybridization (e.g., MERFISH, seqFISH) and proximity ligation-based chromosome-conformation-capture (e.g., 3C, Hi-C), have revealed various chromatin organizations, including kilobase (kb)-scale loops, megabase (Mb)-scale topological associated domains (TADs), and chromosomal compartments. Beyond these ‘static’ structures, dynamic chromatin communication, i.e. dynamic interaction between DNA elements, plays a critical role in genome functions such as stimulus-mediated transcription, DNA damage repair, and epigenetic remodeling. However, since these techniques capture cells at a fixed time point, they limit our understanding of how real-time chromatin communication influences transcription and cellular processes in living cells and tissues.

Imaging specific chromatin regions in living cells requires a method that recruits sufficient fluorescent labels to achieve a high signal-to-noise ratio (SNR) at the genomic site while preserving the integrity of the 3D organization. Additionally, high spatial and temporal resolution is essential for capturing meaningful chromatin dynamics on different timescales, a challenge for current live-cell chromatin imaging approaches. For instance, distinguishing different models of enhancer-promoter interaction requires a spatial resolution of 30 nm or better. Crucial early live-cell imaging systems addressed this by integrating large artificial arrays such as the lac operator (LacO) into specific genomic regions, which were bound by the lac repressor (LacI)-GFP for the recruitment of sufficient fluorophores over the background to visualize the locus. However, this system relies on generating stable cell lines via laborious genomic insertion of large arrays at each target locus, which is challenging in many cell types. Furthermore, introducing large exogeneous tags (~10 kb) could potentially disrupt native chromatin organization and function.

CRISPR-based imaging methods, using either fluorescent proteins or fluorophores recruited to the engineered gRNA scaffold, provide programmable platforms for visualizing chromatin DNA in live cells. However, these methods have been mostly limited to repetitive regions. Efforts to tag non-repetitive loci have faced challenges in imaging resolution and robustness. For example, using a dCas9-EGFP approach, some studies required introducing 72 to 1124 lentiviruses encoding different gRNAs tiling the same region. The CRISPR-CARGO employed chimeric gRNA arrays encoding tens of gRNAs to track the dynamics of enhancers and promoters, and another approach used as few as 4 gRNAs with 16 the MS2 binding motifs to image a non-repetitive region to amplify the imaging signal. The recently reported Casilio system claims to image non-repetitive regions with a single gRNA by incorporating a RNA motif that recruits 15 copies of Pumilio/FBF (PUF)-tethered fluorescent proteins. While the robustness of imaging a non-repetitive genomic site using a single gRNA remains a question, these methods require creating stable cell lines, finetuning expression of multiple constructs, and cannot be applied to primary cells. Furthermore, most methods suffer from low SNR and resolution across diverse cell types including live primary cells.

### SUMMARY

Compositions and methods are provided for flexibly imaging and/or perturbing both repetitive and non-repetitive genomic loci and studying chromatin dynamics in live cells for flexibly imaging both repetitive and non-repetitive genomic loci and studying chromatin dynamics in live cells. Approaches for designing effective gRNA oligo pools to achieve non-repetitive loci imaging by combining a pool of in vitro transcribed (IVT) crRNAs with a universal fluorescently labeled trans-acting crRNA (tracrRNA) are provided. Oligo-LiveFISH provides a robust and versatile method for visualizing diverse endogenous genomic loci in real-time across various cell types, which can be applied to study a variety of biological questions such as enhancer-promoter interactions in live cell.

The present disclosure provides methods for generating a list of chromatin accessible target sequences for CRISPR-Cas guide RNAs that target a contiguous genomic region, the method comprising using one or more computer processors to: (1) generate a master list of all possible target sequences in a genomic region of interest for a PAM sequence of interest, wherein each target sequence is 13-20 nucleotides in length; and (2) generate a list of chromatin accessible target sequences from said master list, via: (2)(i) removing any target sequence from the master list that would result in any of the following sequences being contained in a CRISPR-Cas guide RNA that targets said target sequence: UUUU; (G)₁₀, CUCGAG, GCUNAGC, or CCANNNNNNUGG; (2) (ii) removing any target sequence from the master list that has more than 100 off-target sites, wherein an off-target site is a sequence outside of the genomic region of interest that is adjacent the PAM sequence of interest and: (2) (ii) (a) has 0 or 1 mismatch to the target sequence for target sequences that are less than 16 nucleotides in length; or (2) (ii) (b) has 0, 1, or 2 mismatches to the target sequence for target sequences that are more than 15 nucleotides in length; (2) (iii) removing any target sequence from the master list that has an off-target site in an off-target region, wherein an off-target region is a genomic region outside the genomic region of interest that: (2) (iii) (a) is less than 2000 nucleotides in length; and (2) (iii) (b) contains two or more off-target sites as defined in (2) (ii); (2) (iv) after performing (2) (i), (2) (ii), and (2) (iii): removing, in 5′ to 3′ order along the genomic region of interest, any remaining target sequence from the master list that is 3′ of, and within 10 nucleotides of, another remaining target sequence of the master list; and (2) (v) selecting chromatin accessible target sequences from the remaining target sequences of the master list by: (2) (v) (a) providing one or more histone modification and/or ATAC-seq datasets corresponding to the genomic region of interest, wherein the one or more histone modification datasets comprise H3K27 acetylation enrichment peak data; and (2) (v) (b) selecting target sequences in the list within 5000 nucleotides in distance from an H3K27 acetylation peak and/or an ATAC-seq peak for inclusion in the list of target sequences, thereby generating a list of chromatin accessible target sequences for CRISPR-Cas guide RNAs that target a genomic region of interest.

The present disclosure also provides a plurality of in vitro transcribed CRISPR-Cas guide RNAs, wherein each guide RNA comprises: (a) a guide sequence that hybridizes to a target sequence; and (b) a scaffold sequence that can bind to a CRISPR-Cas effector protein, wherein the plurality comprises 3 or more of said CRISPR-Cas guide RNAs that hybridize to different target sequences within a genomic region of interest at least 500 contiguous nucleotides in length.

The present disclosure further provides a CRISPR-Cas guide RNA comprising: (a) a guide sequence that hybridizes to a target sequence; (b) a scaffold sequence that can bind to a CRISPR-Cas effector protein; and (c) a 3′ poly(A) tail, wherein at least one adenosine of the poly(A) tail comprises a heterologous moiety.

The present disclosure also provides a plurality of guide RNA template nucleic acids, each template nucleic acid comprising: (a) a first primer binding domain common to a subset of the plurality of template nucleic acids; (b) an RNA-polymerase promoter; and (c) a sequence encoding a guide RNA, wherein the guide RNA comprises: (i) a guide sequence that hybridizes to a target sequence; and (ii) a sequence common to the plurality of template nucleic acids, wherein the guide sequences encoded in the template nucleic acids hybridize to target sequences within a genomic region of interest at least 500 contiguous nucleotides in length.

In addition, the present disclosure provides methods of labeling a CRISPR-Cas guide RNA, the method comprising: contacting a CRISPR-Cas guide RNA with a poly(A) polymerase and adenosine triphosphate (ATP) molecules, wherein at least one ATP molecule comprises a heterologous moiety, thereby generating a CRISPR-Cas guide RNA comprising a poly(A) tail in which at least one adenosine of the poly(A) tail comprises the heterologous moiety.

The present disclosure also provides methods of preparing a plurality of CRISPR-Cas guide RNAs for targeting a contiguous genomic region, the method comprising: (a) contacting a plurality of subject template nucleic acids (e.g., see above), or a subset thereof, with an RNA polymerase; and (b) transcribing the plurality of template nucleic acids, or a subset thereof, to produce a plurality of in vitro transcribed CRISPR-Cas guide RNAs for targeting a contiguous genomic region.

The present disclosure further provides methods of guiding a plurality of CRISPR-Cas effector complexes to a contiguous genomic region of interest, the method comprising contacting a contiguous genomic region in a target cell with a plurality of CRISPR-Cas effector complexes, wherein each CRISPR-Cas effector complex in the plurality of complexes comprises: (a) a CRISPR-Cas effector protein; and (b) an in vitro transcribed guide RNA that hybridizes to a target sequence within the contiguous genomic region, thereby guiding the CRISPR-Cas effector complex to the contiguous genomic region, wherein the plurality comprises 3 or more of said guide RNAs, and wherein the genomic region of interest is at least 500 contiguous nucleotides in length.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1J: Principle of Oligo-LiveFISH and RNA labeling optimization for imaging non-repetitive genomic regions in live cells. (A) Schematic of Oligo-LiveFISH by delivering a pool of in vitro assembled fRNPs into the live cells for imaging. (B-D) Oligo-LiveFISH imaging of a 29-kb repetitive Chr3q29 locus with 529 repeats (ACAP2-rep) by annealing tracrRNA-Cy3 with purchased crRNA-A488 (B, from IDT) and inhouse in vitro transcribed crRNA (C). (D) Comparison of the fluorescence intensity of labeled DNA locus (tracrRNA-Cy3) shown in the dashed line in (B) and (C). (E) FISH validation of Oligo-LiveFISH for Chr3 ACAP2-rep site using tracrRNA-Cy3: IVT crRNA with 96.9% specificity (=number of colocalized loci/number of total loci detected by Oligo-LiveFISH). n=255 loci, N=75 cells. The colors of FISH probes and Oligo-LiveFISH probes are different to exclude the possibility that the FISH signal is the remaining signal from Oligo-LiveFISH probes. (F) Distribution of number of Chr3 ACAP2-rep loci detected using Oligo-LiveFISH and FISH in U2OS cells. (G) Three color Oligo-LiveFISH of Chr3 ACAP2-rep, Chr13, and ChrX repetitive regions using tracrRNA: dye-crRNA followed by three color FISH to confirm the specificity. The distribution of the number of loci detected by Oligo-LiveFISH and FISH of Chr13 and ChrX is shown below and of Chr3 is shown in FIG. 8A. The specificities of Chr13 and ChrX repetitive sites are 95.0% and 92.5% respectively. n=101 and 53 loci for Chr13 and ChrX. N=33 and 56 cells for Chr13 and ChrX. (H) Twocolor imaging of ACAP 2-rep regions using A488-crRNA: tracrRNA and IVT dye-sgRNA with 100% colocalization. sgRNA was in vitro transcribed and then labeled with Cy3 at 3′ end using poly(A) polymerase. The distribution of the number of loci detected by Oligo-LiveFISH is shown below. n=191 loci, N=66 cells. (I) Illustration of using in-house in vitro transcribed crRNA pools, annealed with a universal dye-tracrRNA to tile the non-repetitive region. (J) Two-color imaging of a ACAP2-rep region using sgRNA-3′-Cy5 and its upstream ACAP2-nonrep region tiled by 24 crRNAs annealed with a universal Cy3-tracrRNA. An inset of the selected locus in the figure is shown on the top without nucleus channel. Scale bars, 10 μm for all whole nucleus images and lum for the inset in (J).

FIG. 2A-2H: Investigation of the design principles for non-repetitive Oligo-LiveFISH imaging. (A) Schematic of the position of a repetitive Chr3q29 region (529 repeats) and its upstream non-repetitive region near ACAP-2 gene. We designed 192 unique crRNA: tracrRNA in 8 subgroups (P1-P8) to tile the non-repetitive region. (B) Box-and-whisker plot of SNR using different 24 crRNA: tracrRNA probes to tile the non-repetitive region. Each dot represents one locus. n.s., not significant (p>0.05); ****, p<0.0001; unpaired two-tailed t-test for SNR between groups. The boxes show the 25% to 75% interquartile range with the bar indicating the median and the whiskers extend to 10% and 90%, with individual data points shown for the lowest and highest 10% of each dataset. The detection ratio in each group, defined as the number of loci whose SNR>4 over the total number of loci, are shown below. (C) Scatter plot of the mean SNR value or detection ratio and the mean ATAC-seq or mean H3K27ac intensity for all the probes in each group. Each dot represents the average value from all loci in one experimental condition shown in FIG. 2B and FIG. 10B. Color indicates different number of probes. (D) An elastic net regularized linear regression machine learning model for predicting the parameters affecting Oligo-LiveFISH imaging efficiency. We selected features including the number of probes, GC content, length and secondary structure of crRNA, and epigenomic profiles such as ATAC-seq intensity, distance to H3K27ac peak, H3K4mel intensity and H3K4me3 intensity. (E-F) The calculated relative importance of each feature estimated by the elastic net regularized model for predicting SNR (E) and detection ratio (F). (G-H) The performance of the elastic net regularized linear regression model for predicting SNR (G) and detection ratio. (H) using selected features. 17 samples were used to train the model and 2 samples were used to evaluate the performance of the model. The Pearson correlation coefficient r and its significant values p are shown.

FIG. 3A-3M: Oligo-LiveFISH in various chromatin sites and diverse cell types. (A) Schematic of the position of a repetitive Chr13 region (358 repeats) and its downstream non-repetitive TUBGCP3 gene region. (B) An example U2OS cell of two-color imaging of a repetitive Chr13 locus using sgRNA-3′-Cy5 and non-repetitive TUBGCP3 gene region using crRNA: Cy3-tracrRNA. Time lapse image of allele 1 with 10 s per frame is shown on the right. The distance between the repetitive and non-repetitive regions in allele 1 and 2 is shown in FIG. 12A. (C) Schematic of the position of a repetitive ChrX region (666 repeats) and its downstream non-repetitive PLCXD1 gene region in HeLa cells. (D) An example Hela cell of two-color imaging of a repetitive ChrX locus using sgRNA-3′-Cy5 and its downstream non-repetitive PLCXD1 gene region using crRNA: Cy3-tracrRNA. (E) Relative fluorescence intensity of the locus shown in the dashed line in (D). (F) FISH confirmed that most U2OS cells have only one ChrX. (G) Schematic of the position of a FOS gene promoter and its upstream FOS gene enhancer region. (H) Oligo-LiveFISH using Cy3-sgRNA and subsequent FISH validation for FOS gene promoter. (I-J) Two color imaging of FOS gene promoter labeled by crRNA: A488-tracrRNA and its upstream enhancer region labeled by crRNA: Cy3-tracrRNA. Time lapse images of the loci in the box region with 10 s per frame are shown in (J). (K) Schematic of the position of a MYC gene promoter and its upstream enhancer region. (L) Oligo-LiveFISH using crRNA targeting MYC promoter region annealed with a universal Cy3-tracrRNA. (M) Oligo-LiveFISH using crRNA targeting MYC enhancer region annealed with AF647-sgRNA. Scale bars, 10 μm for the whole nucleus image and 1 μm for the time lapse image in (B), (J).

FIG. 4A-4K: Oligo-LiveFISH in primary human T cells and human embryonic stem cells (hESCs) induced neurons (iNs). (A) Schematic of the position of Chr3 ACAP2-rep region (529 repeats) and its upstream Chr3 ACAP2-nonrep region. (B) Two-color imaging of Chr3 ACAP2-rep region using sgRNA-3′-A488 and ACAP 2-nonrep region using 96 crRNA: Cy3-tracrRNA in human primary T cell. Time lapse images of the loci in the box region with 20 s per frame are shown on the right. (C) Two-color imaging of Chr3 ACAP2-rep and ACAP 2-nonrep regions with 98.8% colocalization in primary human T cells. n=94 loci, N=54 cells. (D-E) An example of doublet loci in U20S cell (D) and primary T cell (E) Time lapse images of the doublet loci in the box region are shown on the right. (F) Comparison of the fluorescence intensity of the doublet loci shown in the dashed line in (E). (G) 2D distance between the two loci of the doublet in (E) over time. (H) Fraction of doublet loci in total number of detected loci in primary human T cells and U20S cells. n=98 loci and 297 loci for T cells and U2OS cells. (I) Histogram depicting VyBrant DyeCycle Ruby (left) and CellTrace Far Red (right) fluorescence as measured by flow cytometry. T cells are representative of one donor. (J) Schematic of the position of a FOS gene promoter and its upstream FOS gene enhancer region. (K) Two color imaging of FOS gene promoter labeled by crRNA: Cy3-tracrRNA and its upstream enhancer region labeled by sgRNA-5′-AF647 in human embryonic stem cell. Scale bars, 10 μm for the whole nucleus images and 1 μm for the time lapse images in (B), (D), (E).

FIG. 5A-5L: Oligo-LiveFISH with 2D and 3D super localization microscopy. (A) Principle of superlocalization (SL) microscopy. The intensity profile of the chromatin region that we target (usually <10 kb), can be approximated as a diffraction-limited spot with the width determined by the point spread function (PSF) of the microscope. By fitting the measured fluorescence intensity profile to a 2D Gaussian function, the localization uncertainty (10-20 nm) of the centroid position of DNA loci becomes much smaller than the width of the diffraction-limited PSF (200-300 nm) of the microscope. (B) Diffraction-limited (DL) image and super-localization (SL) image of a Chr3 ACAP2-rep locus. Right: a zoom-in version of the selected locus in the figure. Calibration bar in DL image is the fluorescence intensity (a.u.) and in SL image is the count of localizations in successive frames. (C) Single-molecule bleaching experiment to characterize the number of bound Oligo-LiveFISH fRNPs in each site. Scatter plot of the number of bound fRNPs in the non-repetitive FOS gene promoter using 237 crRNA probes to tile it and in a Chr3 ACAP2-rep site (×529 repeats). There are 44±44 (mean±1×S.D.) (n=29 loci) bound fRNP for the FOS gene promoter and 194±110 (mean±1×S.D.) bound fRNPs for the Chr3 ACAP2-rep site (n=41 loci). Line indicates mean±1×S.D. Bound fraction, defined as the number of bound fRNP over the total available binding sites, is shown on the left. (D) Probability distribution of localization uncertainty for Chr3 ACAP 2-rep loci using 50 ms exposure time (13.6±6.4 nm, mean±1×S.D., n=12951 localizations), Chr3 ACAP2-nonrep loci using 50 ms exposure time (22.2±7.1 nm, mean±1×S.D., n=7700 localization) and FOS gene promoter using 18 ms exposure time (29.0±3.4 nm, mean±1×S.D., n=268 localizations,). (E) Illustration of different timescales associated with chromatin dynamics. (F) An example cell with labeled FOS promoter loci tracked at 8.2 s per frame. Time-lapse images of locus in the box region with 8.2 s per frame is shown on the right. (G-H) Super-fast tracking of non-repetitive FOS promoter region with 18 ms per frame. (G) An example cell with two FOS promoter loci. Time dependent images of locus 2 in the box region with 18 ms per frame is shown on the right. (H) Time trajectory of the two loci in (G). Color indicates time. (I) Principle of 3D double-helix point spread function (DHPSF) imaging. The rotation angle of the lobes reflects the z position of the locus over a large axial range. (J-L) 3D tracking of FOS promoter with 50 ms per frame using DHPSF. The example cell is shown in (J) with 3D trajectory in (K). The time-dependent variation of the position in (x,y,z) directions is shown in (L). Scale bars: 500 nm and 50 nm for DL and SL inset images in (B); 1 μm in the inset images in (F), (G) and (I); 10 μm in the whole nucleus images.

FIG. 6A-6K: Two-color 2D and 3D Oligo-LiveFISH tracking of two sites and biophysical analysis. (A) Illustration of the position of two sites on Chr3: site 1 in 195.46 Mb and site 2 in 195.49 Mb. We track sites 1 and 2 simultaneously with 50 ms per frame in both 2D (B-G) and 3D (H-J). (B) Representative trajectories of sites 1 and 2 in two alleles from the same cell. Color indicates time. (C) The time-dependent variation of the position in X and Y of sites 1 and 2 in two alleles in (B). There is strong correlation in their motion between site 1 and site 2 (28 kb apart). (D) 2D distance between site 1 and site 2 versus time for two alleles in (B). (E) Mean square displacement (MSD), corrected for localization error, for site 1 and site 2 from all trajectories. Orange curve is the fit to Eq. 5 in Methods. Both sites show sub-diffusive behavior. (F) Normalized velocity auto correlation (VAC) function for 2D trajectories for site 2 as a function of lag time t (left) or rescaled by 8 (right) with 8 ranging from 0.05 s to 2.5 s in 0.05 s intervals indicated by the color bar. The blue line on the right is the theoretical prediction of fBM using Eq. 9 and a=0.38. (G) Normalized experimental and theoretical velocity cross correlation (VCC) function between site 1 and site 2 as a function of rescaled lag time t/8 for five 8 values ranging from 0.05 s to 2.5 s. The experimental VCC is calculated from the Oligo-LiveFISH trajectories. The theoretical VCC is calculated from a viscoelastic Rouse polymer model with Rouse relaxation time (communication time) TR (An) as a fitting variable to fit the experimental VCC. Mean squared error between the experimental and theoretical VCC TR (An) as variable is shown on the bottom left. TR (An-28 kb) is found to be 0.44 s for the VCC shown here, which is the with TR (An) that has minimum mean squared error between experimental and theoretical VCC. (H-J) Representative images (H) and trajectories (I) of site 1 and site 2 tracked at 50 ms per frame for ~1500 frames in 3D by DHPSF. The projections of the trajectories in xy, xz, yz planes are also shown in (J). (J) 3D distance between sites 1 & 2 over time for loci pair in (I). The raw distance is shown in black and 10-frame (0.5 s) sliding mean distance is shown in orange. (K) 2D vs 3D distance distributions between site 1 and site 2 from all trajectories. The mean distance is 75.5±57.3 nm (mean±S.D., n=4444 localizations) in 2D and 101.3±44.1 nm (mean±S.D., n=4834 localizations) in 3D. Scale bars, 1 μm in (H).

FIG. 7A-7L: Multi-color Oligo-LiveFISH tracking for different sites reveals two modes of chromatin interaction. (A) Illustration of the position of four sites on Chr3: site 1 at 195.46 Mb, site 2 at 195.49 Mb, site 3 at 195.7 Mb and site 4 at 198.1 Mb. We focus on dynamical organization of sites 2,3 and 4, whose fRNPs are labeled with -A488 (green), -A565 (yellow), -A647 (red) respectively. (B-I) Simultaneous three color tracking of sites 2,3 and 4 on Chr3. (B) A representative image of the three Chr3 alleles (loci groups) in one U2OS cell. Each allele has three sites. (C) Time lapse image of allele 1 and 2 in (B) with 24 seconds per frame. For allele 1, the proximal loci pairs 2 and 3 are closer in spatial position, therefore assigned to the canonical group. For allele 2, the distal loci pairs 3 and 4 are closer in spatial position and therefore assigned to the variant group. Z-projected images are shown in (B-C). (D) 3D Pair-wise distances of sites 2 & 3, sites 2 & 4 and sites 3 & 4 in allele 1 and 2 over time. (E) Scatter plot of the mean 3D distance between sites 2 & 3 (proximal pair) and 3 & 4 (distal pair) of the trajectories (N=67 trajectory groups). Each trajectory group has 3 trajectories with each one corresponding to one site. (F) Scatter plot of the 3D distance between sites 2 & 3 (proximal pair) and 3 & 4 (distal pair) per localization. We chose a cut off distance of spatial proximity of 600 nm. 53% of sites 2 & 3 and 27% of sites 3&4 are in spatial proximity. (G) The 3D inter-locus distance probability distribution between each site in canonical (left) and variant (right) group. In the canonical group, the 3D inter-locus distances between sites 2 & 3, sites 3 & 4, sites 2 & 4 (right y-axis) are 430±242 nm, 1141±491 nm, and 1214±531 nm respectively (mean #1S.D., n=1111, 638, 676 localizations). In the variant group, the inter-locus distance between sites 2 & 3, sites 3 & 4, sites 2 & 4 (right y-axis) are 1167±425 nm, 662±276 nm, and 1425±536 nm (mean±1S.D., n=575, 379, 414 localizations). The 3D distance between sites 1 & 2, adopted from FIG. 6L, is also shown as reference (left y-axis). (H) Scatter plot of mean square 3D inter-locus distance <r2> (mean±1S.D.) with genomic distance in canonical (left) and variant (right) groups. For canonical group, line indicates linear fit, with the slope=(6.70±0.06)×10⁻⁴ μm²/kb (mean±1S.D.). Typical topologically associating domains (TAD) size (40 kb-3 Mb) is shown. (I) The mean squared error between experimental and theoretical 2D VCC between sites 2 & 3 and sites 3 & 4 in canonical and variant groups. The magnified region of the left figure with communication time between 0-500 s is shown on the right. The minimum value of each curve indicates the communicate time TR (An) between two loci, as indicated by the dash line. (J) The Hi-C map (GSM4194449) of Chr3 generated from Juicebox at 5 kb resolution. The four sites are labeled. (K) The relationship between genome-averaged contact probability P(s) and genomic communication distance, adopted from previous simulations with steady-state extrusion. (L) Cartoon illustrating two modes of interaction in live chromatin: the 1D cis-communication, through the elastic property of chromatin polymer and 3D trans-communication, through the linking protein complex. Scale bars, 10 μm in (B) and 1 μm in (C).

FIG. 8A-8G: Specificity of Oligo-LiveFISH using dye-crRNA: tracrRNA and dye-sgRNA, Related to FIG. 1. (A) Principle of sequential Oligo-LiveFISH-FISH to confirm Oligo-LiveFISH specificity. (B) FISH validation of tracrRNA: crRNA-dye targeting the Chr3 ACAP2-rep site with 95.9% specificity (specificity=number of colocalized loci/number of total loci detected by Oligo-LiveFISH). n=97 loci, N=36 cells. The distribution of the number of loci detected by Oligo-LiveFISH and FISH is shown below. (C) Labeling telomere in live cells by transfecting U2OS cells with a plasmid expressing telomere binding protein TRF1-HaloTag and sequential delivery of Oligo-LiveFISH probe targeting telomere. The signals using two methods colocalized, indicating the specificity of Oligo-LiveFISH. (D) Oligo-LiveFISH imaging of Chr3 ACAP2-rep loci using IVT dye-sgRNA. sgRNA was in vitro transcribed and then labeled with Cy3 at 5′ end using (1-ethyl-3-(3-dimethylamino) propyl carbodiimide, hydrochloride) (EDC) mediated reaction. (E) The chemical mechanism of labeling RNA with fluorophore at 5′-end using (1-ethyl-3-(3-dimethylamino) propyl carbodiimide, hydrochloride) (EDC) mediated reaction (F) Two-color imaging of Chr3 ACAP2-rep loci using crRNA-3′-Cy3 annealed with either unlabeled tracrRNA (top) or Cy5-5′-tracrRNA (bottom). (G) Twocolor imaging of a repetitive Chr13 locus using A565-crRNA: tracrRNA and dye-sgRNA. The sgRNA was in vitro transcribed and then labeled with Cy5 at 3′-end using poly(A) polymerase. Scale bars, 10 μm.

FIG. 9A-9G: Control experiments to confirm the specificity of Oligo-LiveFISH, Related to FIG. 1. (A) Imaging Chr3 ACAP2-rep loci using crRNA: Cy3-tracrRNA at different concentrations. The control group with Hoechst only indicates that Hoechst signal has no leakage into the Cy3 (561 nm) channel. The control group with dCas9 only has no detectable loci signal. The control group in 1 pmol and 3 pmol crRNA: Cy3-tracrRNA without dCas9 has no detectable loci signal in the nucleus. Inset, calibration bar of signal intensity. (B) Increasing concentration of Cy3-tracrRNA with non-targeting crRNA results in increasing diffuse background but no detectable loci signal inside the nucleus (white dash line). (C) Oligo-LiveFISH labeling of Chr3 ACAP2-nonrep gene region using 96 crRNA: Cy3-tracrRNA without labeling the repetitive site (D) Two color imaging of Chr3 ACAP2-nonrep loci using 96 crRNAs: Cy3-tracrRNA with dCas9-EGFP. (E) Comparison of the fluorescence intensity in crRNA: Cy3-tracrRNA channel (561 nm) and dCas9-EGFP channel (488 nm) of the ACAP 2-nonrep region in the dashed line in (D). (F) Two color imaging of Chr3 ACAP2-rep loci using crRNA: Cy3-tracrRNA with dCas9-EGFP. (G) Two-color imaging of Chr3 ACAP2-rep region using crRNA-A488: tracrRNA and its upstream Chr3 ACAP2-nonrep region using crRNA: Cy3-tracrRNA. The 136 crRNAs targeting Chr3 ACAP2-nonrep region are generated from a DNA pool ordered from Twist Bioscience. Scale bars, 10 μm.

FIG. 10A-10F: Quantification of SNR in different conditions, Related to FIG. 2. (A) Scatter plot of the SNR in repetitive and non-repetitive color channels in each ROI in one experimental condition (24 gRNAs, group P7 with 18-20 bp in spacer length). Each dot represents one pair of Chr3 ACAP 2-rep and ACAP 2-nonrep genomic regions. (B) Box-and-whisker plot of SNR using different probes to tile the Chr3 ACAP2-nonrep region. Each dot represents one locus. n.s., not significant (p>0.05); ***, p<0.001; ****, p<0.0001; unpaired t-test for SNR between groups. The boxes show 25% to 75% with the bar indicating the median and the whiskers extend to 10% and 90%, with individual data points shown for the lowest and highest 10% of each dataset. The detection ratio in each group, defined as the number of loci whose SNR>4 over the total number of loci, are shown below. (C-F) Scatter plot of the mean SNR value and detection ratio in the group using 12 gRNAs (C), 24 gRNAs (D), 48 gRNAs (E), 96 gRNAs (F), and its mean ATAC-seq value and H3K27ac value. For (C)-(F), each dot represents the average value from all loci in one experimental condition shown in FIG. 2(B) and FIG. 10(B). For (C)-(F), Pearson correlation coefficient r and p value from two-tailed t-test are shown in the figure.

FIG. 11A-11L: Machine learning model, Related to FIG. 2. (A) Heatmap of the correlation between different features in the elastic net regularized linear regression machine learning model. We examined 15 features with nonzero coefficients including: number of distinct gRNA species, the average GC percentage in the spacer sequence, the average length of crRNA, the average secondary structure scores of crRNAs, the average secondary structure scores of crRNA: tracrRNA, the average distance of the target regions to the closest H3K27ac peak, the average distance of the target regions to the closest ATAC peak, average H3K27ac signal, average ATAC signal, average H3K4melsignal, average H3K18ac signal, average H3K56ac signal, average H3K9ac signal, average H3K9me3 signal, and average H3K4me3 signal. (B) The calculated relative importance of each feature estimated by the elastic net regularized model for predicting SNR using all features in (A). (C) Performance of the elastic net regularized linear regression model for predicting SNR using all the features in (A). 17 samples were used to train the model and 2 samples were used to evaluate the performance of the model. The Pearson correlation coefficient (r) and its significant values are shown. Using all features for fitting does not perform as well as using selected features. (D) Schematic showing the difference between signal-to-noise ratio (SNR) and signal-to-background noise ratio (SBN). (E-F) Scatter plot of the mean SBN value and the mean ATAC-seq (E) or mean H3K27ac (F) intensity for all the probes in each group. Each dot represents the average value from all loci in one experimental condition shown in FIG. 2B and FIG. 10B. Color indicates different number of probes. (G-J) The calculated relative importance of each feature estimated by the elastic net regularized model for predicting SBN using selected features (G), SBN using all features (H), detection ratio using selected features (I), and detection ratio using all features (J). (K-L) The performance of the elastic net regularized linear regression model for predicting SBN (K) and detection ratio (L) using selected features. 17 samples were used to train the model and 2 samples were used to evaluate the performance of the model. The detection ratio in (I), (J) and (L) is defined as the fraction of loci whose SBN>5.

FIG. 12A-12H: Oligo-LiveFISH for different genes and different cell types, including primary human T and hESC induced neurons, Related to FIG. 3-4. (A) 3D pair-wise distance between Chr13 repetitive and non-repetitive TUBGCP3 gene over time for the two alleles in FIG. 3B. (B) Schematic of the position of a repetitive Chr3q29 region (529 repeats) and its downstream MUC4 gene non-repetitive region. (C) Time lapse images of the repetitive Chr3q29 region labeled by sgRNA-3′-Cy5 and non-repetitive MUC4 gene labeled by crRNA: Cy3-tracrRNA with 10 sec per frame. (D) 3D pair-wise distance between Chr3 repetitive and non-repetitive MUC4 gene over time for the two loci in (C). (E) The distribution of the number of loci of Chr3 repetitive and non-repetitive ACAP2 regions in activated primary human T cells detected by Oligo-LiveFISH. (F-G) Sample gating strategy for flow cytometry. Samples were gated for live cells(lymphocytes), then single cells. Samples were then compared for Vybrant DyeCycle Ruby fluorescence (F) and CellTrace Far Red fluorescence (G), as indicated. The left two panels are representative of primary human T cell samples and the right two panels are representative of U20S cell samples. (H) Example images of Chr3 ACAP2-rep sites in human embryonic stem cell (hESC) induced neurons (iNs) 6 h and 24 h after electroporation of fRNP. The dendrites and axons indicate the differentiation and normal growth of iN is not affected by Oligo-LiveFISH. Scale bars, 10 μm.

FIG. 13A-13P: Dynamic tracking and analysis of chromatin and beads using 2D and 3D superlocalization Oligo-LiveFISH at different timescales, Related to FIG. 5-6. (A) An example single-molecule bleaching curve and representative image of a FOS promoter locus. There are 91.7 bound fRNPs for this example locus. (B) An example cell with FOS gene promoter labeled with 5′-Cy3-sgRNA tracked with 2.2 sec per frame. (C) An example time-dependent trajectory of FOS gene promoter labeled with crRNA: Cy3-tracrRNA tracked with 50 ms per frame. Color indicates time. (D) Mean square displacement (MSD) of the FOS gene promoter tracked at 18 ms per frame. Black curve is the fitting curve by Eq. 5 in Method. (E) The calibration between the lobe angle and the axial z position in the DHPSF microscope. (F) The time-dependent variation of the position in X and Y directions of one example trajectory of Tetraspeck beads (probe0 in 647 nm and probel in 561 nm) using the same setting as FIG. 6C. (G) Standard deviations of the positions in X and Y directions of trajectories of Tetraspeck beads. Each dot represents S.D. of one trajectory. The standard deviations in both directions of beads tracking are ~20 nm, indicating that the correlated 2D chromatin motion of sites 1 & 2 are not due to stage drift. (H) The time-dependent variation of the centroid position of nucleoid using the same setting as FIG. 6C. Small variation in nucleoid position indicates that the correlated 2D chromatin motion of sites 1 & 2 are not due to the nucleus movement. (I) Representative trajectories of sites 1 & 2 tracked at 50 ms per frame for ~1500 frames in 3D by DHPSF. The projections of the trajectories in xy, xz, yz planes are also shown. (J) The time-dependent variation of the position in (X,Y,Z) directions of sites 1 & 2 in FIG. 6I. (K) The time-dependent variation of the position in (X,Y,Z) directions of one example trajectory of Tetraspeck beads (probe0 in 647 nm and probel in 561 nm) using the same setting as FIG. 13I-13J. (L) Standard deviations of the positions in (X,Y,Z) directions of trajectories of Tetraspeck beads. Each dot represents S.D. of one trajectory. The standard deviations in all directions of beads tracking are ~25 nm, indicating that the correlated 3D chromatin motion of sites 1 & 2 is not due to stage drift. (M) Distribution of the mean distance for each trajectory between sites 1 & 2 (mean±S.D. is 72.3±32.0 nm. N=28 trajectories). (N) Normalized velocity auto correlation (VAC) function for 2D trajectories of site 1 as a function of lag time t with 8 range from 0.05 s to 2.5 s in 0.05 s interval indicated by the color bar. (O) Normalized VAC function for 2D trajectories of beads as a function of rescaled lag time t/8 (right) with & ranging from 0.05 s to 2.5 s in 0.05 s intervals indicated by the color bar. The blue line is the theoretical prediction of fBM using Eq. 9 and a=0.38. No correlation is observed for beads trajectories. (P) Normalized velocity auto correlation function for 3D trajectories of site 1 (left) and site 2 (right) as a function of lag time t with 8 ranging from 0.05 s to 2.5 s in 0.05 s intervals indicated by the color bar. Scale bars, 10 μm.

FIG. 14A-14L: Dynamic analysis of the multi-color tracking of four sites on Chr3, Related to FIG. 7. (A) The 3D inter-locus distance probability distribution between each site in all groups. The mean 3D values for sites 2 & 3, sites 3 & 4, sites 2 & 4 (right y-axis) are 682±471 nm, 963±482 nm, and 1294±542 nm (mean±1SD, n=1686, 1017, 1090 localizations). The 3D distance between sites 1 & 2, adopted from FIG. 6L, is also shown as reference (left y-axis). (B) The distribution of the number of variant loci groups detected in a cell. Only cells with three total detected loci groups are counted here. (C) Gaussian Mixture Model (GMM) with Bayesian Information Criterion (BIC) was applied to fit the distribution of the mean 3D distance between site 2 & 3 (proximal pair) and 3 & 4 (distal pair) of the trajectories (N=67 trajectory groups) by expectation maximization. (Left) BIC value as a function of the number of clusters k in a GMM model. The minimum BIC is obtained when cluster number k=2. (Right) The scatter plot of each data point and the cluster it belongs to. A point was assigned to cluster 1 if the probability of being a member of cluster 1 is larger than 0.5 and otherwise assigned to cluster 2. The centers of the ellipses indicate the means of the individual Gaussian components, and the contours indicate their 1× and 2×S.D. (D) Scatter plot of mean 3D (left) and 2D (right) spatial distance <r> (mean+1S.D.) with genomic distance s in canonical group. Line indicates linear fit in log space, with a scaling exponent 1/d=0.52±0.01 (mean±1S.D.) in 3D and 1/d=0.54±0.01 (mean±1S.D.) in 2D. (E) Scatter plot of mean 3D (left) and 2D (right) spatial distance <r> (mean±1S.D.) with genomic distance s in all groups. Line indicates linear fit in log space, with a scaling exponent 1/d=0.41±0.01 (mean±1S.D.) in 3D and 1/d=0.42=0.01 (mean±1S.D.) in 2D. Typical topologically associating domains (TAD) size (40 kb-3 Mb) is shown. (F) Box-and-whisker plot of mean square 3D inter-locus distance <r2> between each site in canonical group. Each dot represents one loci pair. The boxes show 25% to 75% with the bar indicating the median and the whiskers extend to 10% and 90%, with individual data points shown for the lowest and highest 10% of each dataset. (G) Scatter plot of mean square 2D inter-locus distance <p> (mean±1S.D.) with genomic distance s in canonical and variant groups. For canonical group, line indicates linear fit, with the slope=(6.07±0.06)×10⁻⁴ μm²/kb (mean±1S.D.). Assuming a DNA length of 0.34 nm/bp, this corresponds to a Kuhn length of (1.79±0.02) nm (mean±1S.D.). (H) Mean square change in displacement (MSCD) for the sites 1 & 2 from all trajectories. (I-J) Mean square change in displacement (MSCD) for the three loci pair in canonical group in 2D (I) and in 3D (J). (K) Mean square change in displacement (MSCD) for the three loci pairs in variant group in 2D. (L) The mean squared error between experimental and theoretical 3D VCC between sites 2&3 and sites 3&4 in canonical and variant groups. The minimum value of each curve indicates the communicate time TR (An) between two loci, which is similar as using 2D VCC (data not shown).

FIG. 15A-15P provide amino acid sequences of examples of CRISPR-Cas effector proteins.

### DEFINITIONS

“Heterologous,” as used herein, means a nucleotide or polypeptide sequence that is not found in the native nucleic acid or protein, respectively. For example, relative to a CRISPR-Cas effector polypeptide, a heterologous polypeptide comprises an amino acid sequence from a protein other than the CRISPR-Cas effector polypeptide. As another example, a CRISPR-Cas effector protein (e.g., a dead CRISPR-Cas effector protein) can be fused to an active domain from a non-CRISPR-Cas effector protein (e.g., a cytidine deaminase), and the sequence of the active domain could be considered a heterologous polypeptide (it is heterologous to the CRISPR-Cas effector protein).

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See J. Mol. Biol. 48:443-453 (1970).

“Operatively linked” or “operably linked” refers to a juxtaposition of genetic elements, wherein the elements are in a relationship permitting them to operate in the expected manner. For instance, a promoter is operatively linked to a sequence of interest (the sequence of interest can also be said to be operatively linked to the promoter) if the promoter helps initiate transcription of the sequence of interest. There may be intervening residues between the promoter and sequence of interest so long as this functional relationship is maintained.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (dsRNA duplex) of a guide RNA molecule; of a target nucleic acid base pairing with guide RNA) is considered complementary to both a U and to C. For example, when a G/U base-pair can be made at a given nucleotide position of a protein-binding segment (e.g., dsRNA duplex) of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g. complementarity over 35 or fewer, 30 or fewer, 25 or fewer, 22 or fewer, 20 or fewer, or 18 or fewer nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). The temperature and wash solution salt concentration may be adjusted as necessary according to factors such as length of the region of complementation and the degree of complementation.

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Exemplary methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a CRISP-Cas effector complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. As such, the articles “a” and “an” are used herein to refer to one or to more than one (i.e., to at least one) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods for Generating Lists of Chromatin Accessible Target Sequences for CRISPR-Cas Guide RNAs

The present disclosure provides methods for generating a list of chromatin accessible target sequences for CRISPR-Cas guide RNAs that target a contiguous genomic region, the methods comprising using one or more computer processors to (1): generate a master list of all possible target sequences in a genomic region of interest for a PAM sequence of interest; and (2) generate a list of chromatin accessible target sequences from said master list, via: (i) removing any target sequence from the master list that would result in a CRISPR-Cas guide RNA that is unsuitable for synthesis by in vitro transcription; (ii) removing any target sequence from the master list that has off-target sites; (iii) removing any target sequence from the master list that has an off-target site in an off-target region; (iv) after performing (i), (ii), and (iii): removing, in 5′ to 3′ order along the genomic region of interest, any remaining target sequence from the master list that is 3′ of, and within 10 nucleotides of, another remaining target sequence of the master list; and (v) selecting chromatin accessible target sequences from the remaining target sequences of the master list, thereby generating a list of chromatin accessible target sequences for CRISPR-Cas guide RNAs that target a genomic region of interest. In some embodiments, the methods further comprise producing a plurality of CRISPR-Cas guide RNAs that target sequences from the list of chromatin accessible target sequences generated by the subject methods.

### (1) Generating a Master List of all Possible Target Sequences in a Genomic Region of Interest for a PAM Sequence of Interest

The subject methods for generating a list of chromatin accessible target sequences for CRISPR-Cas guide RNAs that target a contiguous genomic region comprise using one or more computer processors to (1): generate a master list of all possible target sequences in a genomic region of interest for a PAM sequence of interest. The possible target sequences generated for the master list in step (1) can be any desired sequence in the genomic region of interest, with the exception that the protospacer adjacent motif (PAM) sequence of interest is to be taken into account. The PAM sequence of interest may be the PAM sequence for any desired CRISPR-Cas effector protein.

In some embodiments, the possible target sequences generated in the master list of step (1) can have a length of 13 nt to 30 nt (e.g., 13 to 25 nt, 13 to 24 nt, 13 to 23 nt, 13 to 22 nt, 13 to 21 nt, 13 to 20 nt, 13 to 19 nt, 13 to 18 nt, 15 to 25 nt, 15 to 24 nt, 15 to 23 nt, 15 to 22 nt, 15 to 21 nt, 15 to 20 nt, 15 to 19 nt, 15 to 18 nt, 16 to 30 nt, 16 to 25 nt, 16 to 24 nt, 16 to 23 nt, 16 to 22 nt, 16 to 21 nt, 16 to 20 nt, 16 to 19 nt, 16 to 18 nt, 17 to 30 nt, 17 to 25 nt, 17 to 24 nt, 17 to 23 nt, 17 to 22 nt, 17 to 21 nt, 17 to 20 nt, 17 to 19 nt, 17 to 18 nt, 18 to 30 nt, 18 to 25 nt, 18 to 24 nt, 18 to 23 nt, 18 to 22 nt, 18 to 21 nt, 18 to 20 nt, 18 to 19 nt, 19 to 30 nt, 19 to 25 nt, 19 to 24 nt, 19 to 23 nt, 19 to 22 nt, 19 to 21 nt, 19 to 20 nt, 20 to 30 nt, 20 to 25 nt, 20 to 24 nt, 20 to 23 nt, 20 to 22 nt, 20 to 21 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt). In some cases, the possible target sequences have a length of 13 nt. In some cases, the possible target sequences have a length of 14 nt. In some cases, the possible target sequences have a length of 15 nt. In some cases, the possible target sequences have a length of 16 nt. In some cases, the possible target sequences have a length of 17 nt. In some cases, the possible target sequences have a length of 18 nt. In some cases, the possible target sequences have a length of 19 nt. In some cases, the possible target sequences have a length of 20 nt. In some cases, the possible target sequences have a length of 21 nt. In some cases, the possible target sequences have a length of 22 nt. In some cases, the possible target sequences have a length of 23 nt. In some cases, the possible target sequences have a length of 24 nt. In some cases, the possible target sequences have a length of from 13 to 50 nucleotides (e.g., from 13 nucleotides (nt) to 20 nt, from 20 nt to 25 nt, from 25 nt to 30 nt, from 30 nt to 35 nt, from 35 nt to 40 nt, from 40 nt to 45 nt, or from 45 nt to 50 nt). In a particular embodiment, the possible target sequences have a length of 13-20 nt.

(2) (i) Removing any Target Sequence from the Master List that would Result in a CRISPR-Cas Guide RNA that is Unsuitable for Synthesis by In Vitro Transcription.

The subject methods for generating a list of chromatin accessible target sequences for CRISPR-Cas guide RNAs that target a contiguous genomic region further comprise using one or more computer processors to: (2) generate a list of chromatin accessible target sequences from said master list, via: (i) removing any target sequence from the master list that would result in a CRISPR-Cas guide RNA that is unsuitable for synthesis by in vitro transcription. In some embodiments, the removing target sequences from the master list of step (2) (i) comprises removing any target sequence from the master list that would result in any of the following sequences being contained in a CRISPR-Cas guide RNA that targets said target sequence: UUUU; (G) 10, CUCGAG, GCUNAGC, or CCANNNNNNUGG. In some cases, such a sequence would act as an unintended early termination sequence for a Pol-III RNA polymerase (e.g., when expressing from a U6 promoter).

As an example, for an in vitro transcribed guide RNA comprising a protein-binding segment and a guide segment comprising a guide sequence that is complementary to (i.e., targets) said target sequence, and where the protein-binding segment and the guide segment are present on the same nucleic acid molecule, if the combined protein-binding segment and the guide segment sequences would contain: UUUU; (G) 10, CUCGAG, GCUNAGC, or CCANNNNNNUGG, then said target sequence would be removed from the master list. As another example, for an in vitro transcribed guide RNA comprising a protein-binding segment and a guide segment comprising a guide sequence that is complementary to (i.e., targets) said target sequence, and where the protein-binding segment and a guide segment are not present on the same nucleic acid molecule, if the nucleic acid molecule comprising said guide segment would contain: UUUU; (G) 10, CUCGAG, GCUNAGC, or CCANNNNNNUGG, then said target sequence would be removed from the master list.

In some embodiments, the removing target sequences from the master list of step (2) (i) may further comprise removing any target sequence from the master list that would result in a CRISPR-Cas guide RNA that has a GC content that is 40% or less (e.g., 39% or less, 38% or less, 37% or less, 36% or less, 35% or less, 34% or less, 33% or less, 32% or less, 31% or less, or 30% or less) or a GC content that is 75% or more (e.g., 76% or more, 77% or more, 78% or more, 79% or more, 80% or more, 81% or more, 82% or more, 83% or more, 84% or more, or 85% or more). As an example, for an in vitro transcribed guide RNA comprising a scaffold sequence and a guide sequence that is complementary to (i.e., targets) said target sequence, where the scaffold and guide sequences are present on the same nucleic acid molecule, if the nucleic acid molecule has a GC content that is 40% or less or a GC content that is 75% or more, then said target sequence would be removed from the master list. As another example, for an in vitro transcribed guide RNA comprising a scaffold sequence and a guide sequence that is complementary to (i.e., targets) said target sequence, where the scaffold and guide sequences are not present on the same nucleic acid molecule, if the nucleic acid molecule comprising said guide sequence has a GC content that is 40% or less or a GC content that is 75% or more, then said target sequence would be removed from the master list.

(2) (ii) Removing any Target Sequence from the Master List that would Result in a Guide RNA with Off-Target Sites

The subject methods for generating a list of chromatin accessible target sequences for CRISPR-Cas guide RNAs that target a contiguous genomic region further comprise using one or more computer processors to: (2) generate a list of chromatin accessible target sequences from said master list, via: (ii) removing any target sequence from the master list that has off-target sites. In some embodiments, the removing target sequences from the master list of step (2) (ii) comprises removing any target sequence from the master list that has 75 or more off-target sites (e.g., 80 or more off-target sites, 85 or more off-target sites, 90 or more off-target sites, 95 or more off-target sites, 100 or more off-target sites, 105 or more off-target sites, or 110 or more off-target sites). In some cases, the removing target sequences from the master list of step (2) (ii) comprises removing any target sequence from the master list that has more than 100 off-target sites.

In some embodiments, an off-target site can be defined as any sequence outside the genomic region of interest (and in some cases any sequence at a location other than the intended location, even if it is within the genomic region of interest) that is adjacent to a PAM sequence of interest and has 0, 1, or 2 mismatches for target sequences of the master list that are less than 16 nucleotides in length; or has 0, 1, 2, or 3 mismatches for target sequences of the master list that are more than 15 nucleotides in length. In some cases, an off-target site is defined as any sequence outside the genomic region of interest (and in some cases any sequence at a location other than the intended location, even if it is within the genomic region of interest) that is adjacent to a PAM sequence of interest and has 0, or 1 mismatches for target sequences of the master list that are less than 16 nucleotides in length; or has 0, 1, or 2 mismatches for target sequences of the master list that are more than 15 nucleotides in length.

In a particular embodiment, the removing target sequences from the master list of step (2) (ii) comprises removing any target sequence from the master list that has more than 100 off-target sites, wherein an off-target site is defined as any sequence outside the genomic region of interest (and in some cases any sequence at a location other than the intended location, even if it is within the genomic region of interest) that is adjacent to a PAM sequence of interest and has (a) 0, or 1 mismatches for target sequences of the master list that are less than 16 nucleotides in length; or (b) has 0, 1, or 2 mismatches for target sequences of the master list that are more than 15 nucleotides in length.

(2) (iii) Removing any Target Sequence from the Master List that has an Off-Target Site in an Off-Target Region

The subject methods for generating a list of chromatin accessible target sequences for CRISPR-Cas guide RNAs that target a contiguous genomic region further comprise using one or more computer processors to: (2) generate a list of chromatin accessible target sequences from said master list, via: (iii) removing any target sequence from the master list that has an off-target site in an off-target region.

In some embodiments, an off-target region is defined as a genomic region outside the genomic region of interest that is less than 3000 nucleotides in length (e.g., less than 2500 nucleotides in length, less than 2000 nucleotides in length, less than 1500 nucleotides in length, less than 1000 nucleotides in length); and contains one or more subject off-target sites as described herein (e.g., contains one or more off-target sites, contains two or more off-target sites, contains three or more off-target sites). In some cases, an off-target region is defined as a genomic region outside the genomic region of interest that is less than 2000 nucleotides in length; and contains two or more off-target sites as described herein.

(2) (iv) Removing Remaining Target Sequences from the Master List that are Near Other Remaining Target Sequences of the Master List

The subject methods for generating a list of chromatin accessible target sequences for CRISPR-Cas guide RNAs that target a contiguous genomic region further comprise using one or more computer processors to: (2) generate a list of chromatin accessible target sequences from said master list, via: (iv) after performing (2) (i), (2) (ii), and (2) (iii): removing, in 5′ to 3′ order along the genomic region of interest, any remaining target sequence from the master list that is 3′ of, and within 10 nucleotides of (e.g., within 9 nucleotides of, within 8 nucleotides of, within 7 nucleotides of, within 6 nucleotides of, within 5 nucleotides of, within 4 nucleotides of, within 3 nucleotides of, within 2 nucleotides of, within 1 nucleotide), another remaining target sequence of the master list. By a target sequence being 3′ of, and within 10 nucleotides of, another remaining target sequence, it is meant that the 5′ end of said target sequence is separated from the 3′ end of another remaining target sequence by 10 nucleotides or less (e.g., by 9 nucleotides or less, by 8 nucleotides or less, by 8 nucleotides or less, by 7 nucleotides or less, by 6 nucleotides or less, by 5 nucleotides or less, by 4 nucleotides or less, by 3 nucleotides or less, by 2 nucleotides or less, by 1 nucleotides or less), including when the 5′ end of said target sequence is immediately adjacent to the 3′ end of another remaining target sequence or when said target sequence overlaps with the 3′ end of another remaining target sequence.

(2) (v) Selecting Chromatin Accessible Target Sequences from the Remaining Target Sequences of the Master List

The subject methods for generating a list of chromatin accessible target sequences for CRISPR-Cas guide RNAs that target a contiguous genomic region further comprise using one or more computer processors to: (2) generate a list of chromatin accessible target sequences from said master list, via: (v) selecting chromatin accessible target sequences from the remaining target sequences of the master list. In some embodiments, the generating a list of chromatin accessible target sequences of step (2) (v) includes providing one or more histone modification and/or ATAC-seq datasets corresponding to the genomic region of interest; and selecting target sequences in the list within 5000 nucleotides (e.g., within 4500 nucleotides, within 4000 nucleotides, within 3500 nucleotides, within 3000 nucleotides, within 2500 nucleotides, within 2000 nucleotides, within 1500 nucleotides, within 1000 nucleotides) in distance from a histone modification peak and/or an ATAC-seq peak for inclusion in the list of target sequences.

The histone modification peak and/or an ATAC-seq peaks may be assessed based on the counts of sequencing reads in a histone modification and/or ATAC-seq dataset that map to a particular locus in the genome of interest. In some cases, the histone modification peak and/or an ATAC-seq peaks may be assigned and/or annotated by any suitable method or computational program known in the art. Suitable computational programs for assigning/annotating histone modification peak and/or an ATAC-seq peaks include, without limitation, MACS2, HOMER, and SICER/epic2, ZINBA, F-seq, PeakDEck.

In some embodiments, the one or more histone modification dataset may comprise, without limitation, H3K27 acetylation (H3K27ac) enrichment peak data, H3K4 mono-methylation (H3K4me1) enrichment peak data, H3K18 acetylation (H3K18ac) enrichment peak data, H3K56 acetylation (H3K56ac) enrichment peak data, H3K9 acetylation (H3K9ac) enrichment peak data, H3K9 tri-methylation (H3K9me3) enrichment peak data, and/or H3K4 tri-methylation (H3K4me3) enrichment peak data. In some cases, the histone modification and/or ATAC-seq dataset may be generated by known methods in the art. For example, methods for generating datasets comprising H3K27ac enrichment peak data are described in, e.g., Pei, Jiayi, et al. “H3K27ac acetylome signatures reveal the epigenomic reorganization in remodeled non-failing human hearts.” Clinical epigenetics 12 (2020): 1-18, the disclosure of which is incorporated by reference herein. As another example, methods for generating ATAC-seq datasets are described in, e.g., Grandi, Fiorella C., et al. “Chromatin accessibility profiling by ATAC-seq.” Nature protocols 17.6 (2022): 1518-1552, the disclosure of which is incorporated by reference herein. In some cases, the histone modification and/or ATAC-seq datasets may be previously generated histone modification and/or ATAC-seq datasets. For example, suitable histone modification and/or ATAC-seq datasets are available on the Gene Expression Omnibus (GEO) database (ncbi(.)nlm(.)nih(.)gov/geo/). Exemplary histone modification and/or ATAC-seq datasets from GEO include, without limitation, the GSM3734223, GSM4861712, GSM1709274, GSM1709278, GSM1709280, GSM1709282, GSM4079841, and GSM4133297 GEO datasets.

By a target sequence being within 5000 nucleotides (e.g., within 4500 nucleotides, within 4000 nucleotides, within 3500 nucleotides, within 3000 nucleotides, within 2500 nucleotides, within 2000 nucleotides, within 1500 nucleotides, within 1000 nucleotides) in distance from a histone modification peak and/or ATAC-seq peak, it is meant that at least part of said target sequence occurs within, or overlaps with, the 5000 nucleotides (e.g., 4500 nucleotides, 4000 nucleotides, 3500 nucleotides, 3000 nucleotides, 2500 nucleotides, 2000 nucleotides, 1500 nucleotides, 1000 nucleotides) 5′ of the center of a histone modification peak and/or ATAC-seq enrichment peak or occurs within, or overlaps with, the 5000 nucleotides (e.g., 4500 nucleotides, 4000 nucleotides, 3500 nucleotides, 3000 nucleotides, 2500 nucleotides, 2000 nucleotides, 1500 nucleotides, 1000 nucleotides) 3′ of the center of a histone modification peak and/or ATAC-seq enrichment peak.

In a particular embodiment, the selecting chromatin accessible target sequences from the remaining target sequences of the master list comprises providing one or more histone modification and/or ATAC-seq datasets corresponding to the genomic region of interest, wherein the one or more histone modification datasets comprise H3K27 acetylation enrichment peak data; and selecting target sequences in the list within 5000 nucleotides (e.g., within 4500 nucleotides, within 4000 nucleotides, within 3500 nucleotides, within 3000 nucleotides, within 2500 nucleotides, within 2000 nucleotides, within 1500 nucleotides, within 1000 nucleotides) in distance from an H3K27ac and/or ATAC-seq enrichment peak.

### PAM Sequences

As discussed above, the possible target sequences generated for the master list in step (1) can be any desired sequence in the genomic region of interest, with the exception that the protospacer adjacent motif (PAM) sequence of interest be taken into account. The PAM sequence of interest may be the PAM sequence for any desired CRISPR-Cas effector protein, e.g., any subject PAM sequence as described herein.

In some cases, site-specific targeting of a CRISPR-Cas effector protein to a target nucleic acid occurs at locations determined by both (i) base-pairing complementarity between the guide nucleic acid and the target nucleic acid; and (ii) a short motif referred to as the “protospacer adjacent motif” (PAM) in the target nucleic acid. For example, when a Cas9 protein binds to (in some cases cleaves) a dsDNA target nucleic acid, the PAM sequence that is recognized (bound) by the Cas9 protein is present on the non-complementary strand (the strand that does not hybridize with the targeting segment of the guide nucleic acid) of the target DNA. For Cas9, the PAM is immediately 3′ of the target sequence (i.e., is positioned downstream of the targeted sequence). In some cases, a PAM sequence has a length in a range of from 1 nt to 15 nt (e.g., 1 nt to 14 nt, 1 nt to 13 nt, 1 nt to 12 nt, 1 nt to 11 nt, 1 nt to 10 nt, 1 nt to 9 nt, 1 nt to 9 nt, 1 nt to 8 nt, 1 nt to 7 nt, 1 nt to 6 nt, 1 nt to 5 nt, 1 nt to 4 nt, 1 nt to 3 nt, 2 nt to 15 nt, 2 nt to 14 nt, 2 nt to 13 nt, 2 nt to 12 nt, 2 nt to 11 nt, 2 nt to 10 nt, 2 nt to 9 nt, 2 nt to 8 nt, 2 nt to 7 nt, 2 nt to 6 nt, 2 nt to 5 nt, 2 nt to 4 nt, 2 nt to 3 nt, 2 nt, or 3 nt).

CRISPR-Cas effector proteins (e.g., Cas9) from different species can have different PAM sequence requirements. For example, in some embodiments (e.g., when the CRISPR-Cas effector protein of interest is a Cas9 protein is derived from S. pyogenes or a closely related Cas9; see for example, Chylinski et al., RNA Biol. 2013 May; 10 (5): 726-37; and Jinek et al., Science. 2012 Aug. 17; 337 (6096): 816-21; both of which are hereby incorporated by reference in their entirety), the PAM sequence of interest can be NRG because the S. pyogenes Cas9 PAM (PAM sequence) is NAG or NGG (or NRG where “R” is A or G). For example, a Cas9 PAM sequence for S. pyogenes Cas9 can be: NGG, NAG, AGG, CGG, GGG, TGG, AAG, CAG, GAG, and TAG. In some cases, the PAM is NGG.

In some cases (e.g., when the CRISPR-Cas effector protein of interest is a Cas9 protein derived from the Cas9 protein of Neisseria meningitidis or a closely related Cas9 is used), the PAM sequence of interest can be 5′-NNNNGANN-3′, 5′-NNNNGTTN-3′, 5′-NNNNGNNT-3′, 5′-NNNNGTNN-3′, 5′-NNNNGNTN-3′, or 5′-NNNNGATT-3′, where N is any nucleotide. In some embodiments (e.g., when a Cas9 protein is derived from Streptococcus thermophilus #1 or a closely related Cas9 is used), the PAM sequence (e.g., of a target nucleic acid) can be 5′-NNAGAA-3′, 5′-NNAGGA-3′, 5′-NNGGAA-3′, 5′-NNANAA-3′, or 5′-NNGGGA-3′ where N is any nucleotide. In some embodiments (e.g., when the CRISPR-Cas effector protein of interest is a Cas9 protein is derived from Treponema denticola (TD) or a closely related Cas9), the PAM sequence of interest can be 5′-NAAAAN-3′, 5′-NAAAAC-3′, 5′-NAAANC-3′, 5′-NANAAC-3′, or 5′-NNAAAC-3′, where N is any nucleotide. As would be known by one of ordinary skill in the art, additional PAM sequences for other Cas9 proteins and other CRISPR-Cas effector proteins are known in the art and/or can readily be determined using bioinformatic analysis (e.g., analysis of genomic sequencing data), and/or routine experimentation. See Esvelt et al., Nat Methods. 2013 November; 10 (11): 1116-21, for additional information. In some cases, the PAM sequence for S. aureus Cas9 is 5′-NNGRR (N)-3′ (in some cases 5′-NNGRRT-3′).

For Cas12a proteins, the PAM is immediately 5′ of the target sequence (i.e., is positioned upstream of the targeted sequence) (5′ of the non-complementary strand of the target DNA, where the complementary strand hybridizes to the guide sequence of the guide RNA while the non-complementary strand does not directly hybridize with the guide RNA). For example, the wild type protein of LbCas12a has a PAM preference of 5′-TTTV-3′, where Vis A, C, or G. As such, LbCas12a can utilize the following PAMs: 5′-TTTA-3′, 5′-TTTC-3′, and 5′-TTTG-3′. Example PAMs for wild type Cas12a proteins include: PAM: 5′-TTTV-3′: LbCas12a; PAM: 5′-TTTV-3′: AsCas12a; PAM: 5′-TTN-3′: FnCas12a, PmCas12a, MbCas12a, Mb2Cas12a, Mb3Cas12a, TsCas12a, and BsCas12a; * where N=(A, C, G, or T); V=(A, C, or G); Y=(C or T); K=(G or T); S=(C or G).

### Genomic Regions of Interest

The genomic region of interest for the subject methods may be any desired contiguous genomic region in a genome of interest. In some embodiments, the genomic region of interest is a non-repetitive genomic region, as opposed to, e.g., a repetitive genomic region. In other words, a non-repetitive genomic region can be recognized as any genomic region that is not a repetitive genomic region.

Repetitive genomic regions are characterized by repeated patterns of nucleic acid sequences that occur, sometimes in tandem, in multiple copies throughout a genome. For example, repetitive genomic regions can include, without limitation, tandem repeats, microsatellites, minisatellites, DNA transposons (e.g., MITEs, Mavericks), and RNA transposons (e.g., LTRs, LINEs, SINEs). Repetitive genomic regions are known in the art and described, e.g., in Liao, Xingyu, et al. “Repetitive DNA sequence detection and its role in the human genome.” Communications Biology 6.1 (2023): 954; and Jurka, Jerzy, et al. “Repetitive sequences in complex genomes: structure and evolution.” Annu. Rev. Genomics Hum. Genet. 8.1 (2007): 241-259, the disclosures of which are incorporated by reference herein.

In some embodiments of the subject methods, the genomic region of interest is a genomic region that is at least 500 contiguous nucleotides in length (e.g., at least 1000 contiguous nucleotides in length, at least 2000 contiguous nucleotides in length, at least 3000 contiguous nucleotides in length, at least 4000 contiguous nucleotides in length, at least 5000 contiguous nucleotides in length, at least 10,000 contiguous nucleotides in length, at least 20,000 contiguous nucleotides in length, at least 30,000 contiguous nucleotides in length, at least 40,000 contiguous nucleotides in length, at least 50,000 contiguous nucleotides in length, at least 100,000 contiguous nucleotides in length, at least at least 200,000 contiguous nucleotides in length, at least at least 300,000 contiguous nucleotides in length, at least at least 400,000 contiguous nucleotides in length, at least at least 500,000 contiguous nucleotides in length, at least 1,000,000 contiguous nucleotides in length, at least 1,000,000 contiguous nucleotides in length, at least 1,250,000 contiguous nucleotides in length, at least 1,500,000 contiguous nucleotides in length, at least 1,750,000 contiguous nucleotides in length, or at least 2,000,000 contiguous nucleotides in length).

In some embodiments of the subject methods, the genomic region of interest is 500 to 2,000,000 contiguous nucleotides in length. For example, in some cases, the genomic region of interest is 500 to 5000 contiguous nucleotides (e.g., 1000 to 4500 contiguous nucleotides, 1500 to 4000 contiguous nucleotides, 2000 to 3500 contiguous nucleotides, 2500 to 3000 contiguous nucleotides) in length. In some cases, the genomic region of interest is 5000 to 20,000 contiguous nucleotides (e.g., 6000 to 19,000 contiguous nucleotides, 7000 to 18,000 contiguous nucleotides, 8000 to 17,000 contiguous nucleotides, 9000 to 16,000 contiguous nucleotides, 10,000 to 15,000 contiguous nucleotides, 11,000 to 14,000 contiguous nucleotides, 12,000 to 13,000 contiguous nucleotides) in length. In some cases, the genomic region of interest is 20,000 to 100,000 contiguous nucleotides (e.g., 30,000 to 90,000 contiguous nucleotides, 40,000 to 80,000 contiguous nucleotides, 50,000 to 70,000 contiguous nucleotides, 55,000 to 65,000 contiguous nucleotides) in length. In some cases, the genomic region of interest is 100,000 to 1,000,000 contiguous nucleotides (e.g., 200,000 to 900,000 contiguous nucleotides, 300,000 to 800,000 contiguous nucleotides, 400,000 to 700,000 contiguous nucleotides, 500,000 to 600,000 contiguous nucleotides) in length. In some cases, the genomic region of interest is 1,000,000 to 2,000,000 contiguous nucleotides (e.g., 1,100,000 to 1,900,000 contiguous nucleotides, 1,200,000 to 1,800,000 contiguous nucleotides, 1,300,000 to 1,700,000 contiguous nucleotides, 1,400,000 to 1,600,000 contiguous nucleotides) in length.

The genomic region of interest may a region in any genome of interest. In some embodiments, the genome is the nuclear genome of a eukaryotic cell (e.g., a protist, fungi, plant, and vertebrate eukaryotic cell). In some cases, the genome is a genome of a eukaryotic cell is a mammalian cell (e.g., a mammalian cell of humans; non-human primates; domestic and farm animals; and zoo, laboratory, sports, or pet animals, such as dogs, horses, cats, cows, mice, rats, rabbits, etc.). In some cases, the genome is a genome of a human cell. In some cases, the genome is a mitochondrial or chloroplast genome.

### Lists of Chromatin Accessible Target Sequences for CRISPR-Cas Guide RNAs

As discussed above, the subject methods generate a list of chromatin accessible target sequences for CRISPR-Cas guide RNAs that target a genomic region of interest. In some embodiments, the subject methods may produce a list of at least 3 (e.g., at least 5, at least 10, at least 20, at least 50, at least 100, at least 250, at least 500, at least 1000, at least 2000, at least 5000, at least 10,000, at least 15,000, at least 20,000) chromatin accessible sequences for CRISPR-Cas guide RNAs that target a genomic region of interest.

In some embodiments, the subject methods may produce a list of 3 to 20,000 chromatin accessible target sequences. For example, in some cases, the subject methods may produce a list of 3 to 10 (e.g., 4 to 9, 5 to 8, 6 to 7) chromatin accessible target sequences. In some cases, the subject methods may produce a list of 10 to 100 (e.g., 20 to 90, 30 to 80, 40 to 70, 50 to 60) chromatin accessible target sequences. In some cases, the subject methods may produce a list of 100 to 1000 (e.g., 200 to 900, 300 to 800, 400 to 700, 500 to 600) chromatin accessible target sequences. In some cases, the subject methods may produce a list of 1000 to 10,000 (e.g., 2000 to 9000, 3000 to 8000, 4000 to 7000, 5000 to 6000) chromatin accessible target sequences. In some cases, the subject methods may produce a list of 10,000 to 20,000 (e.g., 11,000 to 19,000, 12,000 to 18,000, 13,000 to 17,000, 14,000 to 16,000) chromatin accessible target sequences.

In some embodiments, the subject methods may produce a list of one chromatin accessible target sequence for every 100 base pairs (bp)-150 base pairs (bp) in the genomic region of interest. In other words, the subject methods may produce list of chromatin accessible target sequences that occur, on average, every 100 bp-150 bp in the genomic region of interest. In some embodiments, the subject methods may produce a list of one chromatin accessible target sequence for every 100 bp-110 bp (e.g., 101 bp-109 bp, 102 bp-108 bp, 103 bp-107 bp, 104 bp-106 bp) in the genomic region of interest. In some embodiments, the subject methods may produce a list of one chromatin accessible target sequence for every 110 bp-120 bp (e.g., 111 bp-119 bp, 112 bp-118 bp, 113 bp-117 bp, 114 bp-116 bp) in the genomic region of interest. In some embodiments, the subject methods may produce a list of one chromatin accessible target sequence for every 120 bp-130 bp (e.g., 121 bp-129 bp, 122 bp-128 bp, 123 bp-127 bp, 124 bp-126 bp) in the genomic region of interest. In some embodiments, the subject methods may produce a list of one chromatin accessible target sequence for every 130 bp-140 bp (e.g., 131 bp-139 bp, 132 bp-138 bp, 133 bp-137 bp, 134 bp-136 bp) in the genomic region of interest. In some embodiments, the subject methods may produce a list of one chromatin accessible target sequence for every 140 bp-150 bp (e.g., 141 bp-149 bp, 142 bp-148 bp, 143 bp-147 bp, 144 bp-146 bp) in the genomic region of interest.

### Computers & Computer Processors

Also provided are one or more computational systems (e.g., a computer or a computer processor) that may be used in the methods of the present disclosure. In some embodiments, a computational system of the present disclosure may be used to (1) generate a master list of all possible target sequences in a genomic region of interest for a PAM sequence of interest, as described herein; and (2) generate a list of chromatin accessible target sequences from said master list, via: (i) removing any target sequence from the master list that would result in a CRISPR-Cas guide RNA that is unsuitable for synthesis by in vitro transcription, as described herein; (ii) removing any target sequence from the master list that has off-target sites, as described herein; (iii) removing any target sequence from the master list that has an off-target site in an off-target region, as described herein; (iv) after performing (i), (ii), and (iii): removing, in 5′ to 3′ order along the genomic region of interest, any remaining target sequence from the master list that is 3′ of, and within 10 nucleotides of, another remaining target sequence of the master list, as described herein; and (v) selecting chromatin accessible target sequences from the remaining target sequences of the master list, as described herein. Each computational system of the present disclosure may perform one or more functions as described above.

A computational unit may include any suitable components to perform the functions as described above. Thus, the computational unit may include one or more of the following: a processor; a non-transient, computer-readable memory, such as a computer-readable medium; an input device, such as a keyboard, mouse, touchscreen, etc.; an output device, such as a monitor, screen, speaker, etc.; a network interface, such as a wired or wireless network interface; and the like. The data of interest, such as the sequence of the genomic region interest, and the like, can be analyzed and stored on a computer-based system. As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present disclosure. The data storage means may comprise any manufacture comprising a recording of the present information as described above, or a memory access means that can access such a manufacture.

Performance of the described functions may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets, lists, and data comparisons of the present disclosure. In some embodiments, the function is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

The computer also comprises a program of instructions. The program of instructions can comprise a machine learning algorithm used for selecting chromatin accessible target sequences from the remaining target sequences of the master list, as described herein. Any machine learning algorithms deemed useful may be used. Useful machine learning algorithms include, without limitation, elastic-net regularized generalized linear models, two-cluster Gaussian mixture models, logistic regression, random forest, gradient boosted tree, support vector machine, linear/quadratic discriminant analysis, k nearest neighbors, naïve bayes, neural network, etc.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein. A variety of structural formats for the input and output means can be used to input and output the information in the computer-based systems of the present invention.

The data and analysis thereof can be provided in a variety of media to facilitate their use. “Media” refers to a manufacture that contains the signature pattern information of the present disclosure. The data of the present disclosure can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present data and information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

Further provided herein is a method of storing and/or transmitting, via computer, sequence, and other, data collected by the methods disclosed herein. Any computer or computer accessory including, but not limited to software and storage devices, can be utilized to practice the present disclosure. Sequence or other data can be input into a computer by a user either directly or indirectly. Additionally, any devices which can be used to sequence DNA or analyze DNA can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

### Producing a Plurality of CRISPR-Cas Guide RNAs

As discussed above, the subject methods generate a list of chromatin accessible target sequences for CRISPR-Cas guide RNAs that target a contiguous genomic region. The term “targets” with respect to a guide RNA can be used to describe 100% complementarity between nucleic acid molecules. As a non-limiting example, a guide RNA that hybridizes (with 100% complementarity) to a particular target sequence within a target DNA (the guide sequence of the guide RNA hybridizes to the target sequence of a target DNA) can be said to “target” that particular sequence. As such, a plurality of guide RNAs can be said to target a set of target sequences if they hybridize to those sequences with 100% complementarity. In some cases, a guide RNA can also hybridize with less than 100% complementarity to another site. For example, a given guide RNA may ‘target’ a first target site to which it has 100% complementarity, but it may also be able to hybridize to another site (a second site) with which it has less than 100% complementarity. The guide RNA would not be considered to ‘target’ the second site, even though it can hybridize with that second site. In some cases, the second site would be considered an off-target site. Off-target sites are discussed elsewhere herein.

In some embodiments, the subject methods further comprise producing a plurality of CRISPR-Cas guide RNAs that target sequences from the list of chromatin accessible target sequences generated by the subject methods.

In some embodiments, the methods comprise producing one or more CRISPR-Cas guide RNAs that target sequences from the list of chromatin accessible target sequences. In some cases, the methods comprise producing one or more CRISPR-Cas guide RNAs that target a subset of the target sequences from the list of chromatin accessible target sequences. In some cases, the methods comprise producing a plurality of CRISPR-Cas guide RNAs that target all the target sequences from the list of chromatin accessible target sequences. In some cases, the plurality of CRISPR-Cas guide RNAs may be any of the subject plurality of in vitro CRISPR-Cas guide RNAs described herein. In some cases, each CRISPR-Cas guide RNA, of the plurality of guide RNAs, may be a subject CRISPR-Cas guide RNA as described herein.

As will be appreciated by the person of skill, the plurality of CRISPR-Cas guide RNAs may be produced by any of the methods known in the art for producing guide RNAs. In some embodiments, the methods of producing the CRISPR-Cas guide RNAs comprise in vitro transcribing a plurality of CRISPR-Cas guide RNAs from a plurality of template nucleic acids. In some cases, the template nucleic acids are single-stranded (e.g., single-stranded oligonucleotides). In some cases, the template nucleic acids are double-stranded (e.g., double-stranded oligonucleotides). In some embodiments, the template nucleic acid may be a subject template nucleic acid as described herein. In some embodiments, the plurality of CRISPR-Cas guide RNAs may be produced according to the subject methods described herein.

### CRISPR-CAS Guide RNAs

A CRISPR-Cas guide RNA can be said to include two segments, a first segment (referred to herein as a “targeting segment”); and a second segment (referred to herein as a “protein-binding segment” or “scaffold” or “common region” or “constant region”). By “segment” it is meant a segment/section/region of a molecule, e.g., a contiguous stretch of nucleotides in a nucleic acid molecule. A segment can also mean a region/section of a complex such that a segment may comprise regions of more than one molecule. The “targeting segment” is also referred to herein as a “variable region” of a guide RNA. The “protein-binding segment” is also referred to herein as a “constant region” or “scaffold sequence” of a guide RNA.

In many instances, the targeting segment and the protein-binding segment are heterologous to one another. The first segment (targeting segment) of a guide RNA includes a nucleotide sequence (a “guide sequence”) that is complementary to (and therefore hybridizes with) a specific target sequence (a target site) within a target nucleic acid (e.g., a target DNA). The protein-binding segment (or “protein-binding sequence”) includes a nucleotide sequence (a “scaffold sequence” or simply “scaffold”) that interacts with (binds to) a CRISPR-Cas effector polypeptide. The protein-binding segment of a guide RNA includes two complementary stretches of nucleotides that hybridize to one another to form a double stranded RNA duplex (dsRNA duplex). Site-specific binding and/or cleavage of a target nucleic acid (e.g., a target sequence of a list of chromatin accessible target sequences generated by the subject methods described herein) is determined by base-pairing complementarity between the guide RNA (the guide sequence of the guide RNA) and the target nucleic acid sequence.

A guide RNA and a CRISPR-Cas effector polypeptide form a complex (e.g., bind via non-covalent interactions). The guide RNA provides target specificity to the complex by including a targeting segment, which includes a guide sequence (a nucleotide sequence that is complementary to a sequence of a target nucleic acid, e.g., a target nucleic acid selected from a list of chromatin accessible target sequences generated by the subject methods). The CRISPR-Cas effector polypeptide of the complex provides the site-specific activity (e.g., cleavage activity or an activity provided by the CRISPR-Cas effector polypeptide when the CRISPR-Cas effector polypeptide is a CRISPR-Cas effector polypeptide fusion polypeptide, i.e., has a fusion partner). In other words, the CRISPR-Cas effector polypeptide is guided to a target nucleic acid sequence (e.g. a target sequence in a target DNA) by virtue of its association with the guide RNA.

The term “CRISPR-Cas guide RNA” (or simply “guide RNA”) is generic to a guide RNA in general. When a particular type of guide RNA is intended, e.g., one that interacts with a particular CRISPR-Cas effector polypeptide (e.g., Cas9), then the effector polypeptide's name is used in the guide RNA name—e.g., a “CRISPR-Cas9 guide RNA” (or simply “Cas9 guide RNA”) is a CRISPR-Cas guide RNA that forms a complex with (and functions with) Cas9. The guide RNA can be any desired CRISPR-Cas guide RNA. For example, in some cases, the guide RNA(s) is a Cas9 guide RNA(s). In some cases, the guide RNA(s) is a Cas12 guide RNA(s). In some cases, the guide RNA(s) is a Cas13 guide RNA(s).

The “guide sequence” also referred to as the “targeting sequence” (or “spacer”) of a guide RNA can be modified so that the guide RNA can target a CRISPR-Cas effector polypeptide to any desired sequence of a target DNA (e.g., a target DNA sequence selected from a list of chromatin accessible target sequences generated by the subject methods), with the exception that the protospacer adjacent motif (PAM) sequence (present on the target DNA) should be taken into account when targeting double stranded DNA.

In some cases, a guide RNA includes two separate nucleic acid molecules (e.g., crRNA and tracrRNA, sometimes referred to as targeter-RNA and activator-RNA, respectively) and is referred to herein as a “dual guide RNA”, a “double-molecule guide RNA”, or a “two-molecule guide RNA” a “dual guide RNA”, or a “dgRNA.” In some cases, a guide RNA is a single-molecule RNA and is referred to as a “single guide RNA”, or simply “sgRNA.”

In some embodiments, the targeting segment has a length of 7 or more nucleotides (nt) (e.g., 8 or more, 9 or more, 10 or more, 12 or more, 15 or more, 20 or more, 25 or more, 30 or more, or 40 or more nucleotides). In some cases, the targeting segment can have a length of from 7 to 100 nucleotides (nt) (e.g., from 7 to 80 nt, from 7 to 60 nt, from 7 to 40 nt, from 7 to 30 nt, from 7 to 25 nt, from 7 to 22 nt, from 7 to 20 nt, from 7 to 18 nt, from 8 to 80 nt, from 8 to 60 nt, from 8 to 40 nt, from 8 to 30 nt, from 8 to 25 nt, from 8 to 22 nt, from 8 to 20 nt, from 8 to 18 nt, from 10 to 100 nt, from 10 to 80 nt, from 10 to 60 nt, from 10 to 40 nt, from 10 to 30 nt, from 10 to 25 nt, from 10 to 22 nt, from 10 to 20 nt, from 10 to 18 nt, from 12 to 100 nt, from 12 to 80 nt, from 12 to 60 nt, from 12 to 40 nt, from 12 to 30 nt, from 12 to 25 nt, from 12 to 22 nt, from 12 to 20 nt, from 12 to 18 nt, from 14 to 100 nt, from 14 to 80 nt, from 14 to 60 nt, from 14 to 40 nt, from 14 to 30 nt, from 14 to 25 nt, from 14 to 22 nt, from 14 to 20 nt, from 14 to 18 nt, from 16 to 100 nt, from 16 to 80 nt, from 16 to 60 nt, from 16 to 40 nt, from 16 to 30 nt, from 16 to 25 nt, from 16 to 22 nt, from 16 to 20 nt, from 16 to 18 nt, from 18 to 100 nt, from 18 to 80 nt, from 18 to 60 nt, from 18 to 40 nt, from 18 to 30 nt, from 18 to 25 nt, from 18 to 22 nt, or from 18 to 20 nt).

In some embodiments, the guide sequence of a guide RNA has a length of 12 nt to 30 nt (e.g., 12 to 25 nt, 12 to 24 nt, 12 to 23 nt, 12 to 22 nt, 12 to 21 nt, 12 to 20 nt, 12 to 19 nt, 12 to 18 nt, 13 to 30 nt, 13 to 25 nt, 13 to 24 nt, 13 to 23 nt, 13 to 22 nt, 13 to 21 nt, 13 to 20 nt, 13 to 19 nt, 13 to 18 nt, 15 to 25 nt, 15 to 24 nt, 15 to 23 nt, 15 to 22 nt, 15 to 21 nt, 15 to 20 nt, 15 to 19 nt, 15 to 18 nt, 16 to 30 nt, 16 to 25 nt, 16 to 24 nt, 16 to 23 nt, 16 to 22 nt, 16 to 21 nt, 16 to 20 nt, 16 to 19 nt, 16 to 18 nt, 17 to 30 nt, 17 to 25 nt, 17 to 24 nt, 17 to 23 nt, 17 to 22 nt, 17 to 21 nt, 17 to 20 nt, 17 to 19 nt, 17 to 18 nt, 18 to 30 nt, 18 to 25 nt, 18 to 24 nt, 18 to 23 nt, 18 to 22 nt, 18 to 21 nt, 18 to 20 nt, 18 to 19 nt, 19 to 30 nt, 19 to 25 nt, 19 to 24 nt, 19 to 23 nt, 19 to 22 nt, 19 to 21 nt, 19 to 20 nt, 20 to 30 nt, 20 to 25 nt, 20 to 24 nt, 20 to 23 nt, 20 to 22 nt, 20 to 21 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt). In some cases, the guide sequence has a length of 11 nt. In some cases, the guide sequence has a length of 12 nt. In some cases, the guide sequence has a length of 12-19 nt. In some cases, the guide sequence has a length of 12-18 nt. In some cases, the guide sequence has a length of 13 nt. In some cases, the guide sequence has a length of 14 nt. In some cases, the guide sequence has a length of 15 nt. In some cases, the guide sequence has a length of 16 nt. In some cases, the guide sequence has a length of 17 nt. In some cases, the guide sequence has a length of 18 nt.

In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt. In some cases, the guide sequence has a length of 24 nt. In some cases, the guide sequence has a length of from 13 to 50 nucleotides (e.g., from 13 nucleotides (nt) to 20 nt, from 20 nt to 25 nt, from 25 nt to 30 nt, from 30 nt to 35 nt, from 35 nt to 40 nt, from 40 nt to 45 nt, or from 45 nt to 50 nt). In a particular embodiment, the guide sequence has a length of 13-20 nt.

In some cases, a guide RNA may include one or more A-U flips that may, for example, improve the utility of the guide RNA for imaging applications. A-U flips are known in the art and described, e.g., in Chen, Baohui, et al. “Dynamic imaging of genomic loci in living human cells by an optimized CRISPR-Cas system.” Cell 155.7 (2013): 1479-1491, the disclosure of which is incorporated by reference herein.

In some cases, the nucleic acid molecules of the guide RNA may begin with G which may, for example, improve the production of said guide RNAs via in vitro transcription.

### In Vitro Transcribed CRISPR-CAS Guide RNAs

The present disclosure provides a plurality of in vitro transcribed CRISPR-Cas guide RNAs, each guide RNA comprising: (a) a guide sequence that hybridizes to a target sequence; and (b) a scaffold sequence that can bind to a CRISPR-Cas effector protein, wherein the plurality comprises 3 or more (e.g., 5 or more, 10 or more, 20 or more, 50 or more, 100 or more, 250 or more, 500 or more, 1000 or more, 2000 or more, 5000 or more, 10,000 or more, 15,000 or more, 20,000 or more) of said CRISPR-Cas guide RNAs that hybridize to different target sequences within a genomic region of interest at least 500 (e.g., at least 1000, at least 2000, at least 3000, at least 4000, at least 5000, at least 10,000, at least 20,000, at least 30,000, at least 40,000, at least 50,000, at least 100,000, at least at least 200,000, at least at least 300,000, at least at least 400,000, at least at least 500,000, at least 1,000,000, at least 1,000,000, at least 1,250,000, at least 1,500,000, at least 1,750,000, or at least 2,000,000) contiguous nucleotides in length. In some embodiments, the in vitro transcribed CRISPR-Cas guide RNAs may be produced according to the subject methods described herein.

In some embodiments, the plurality of in vitro transcribed CRISPR-Cas guide RNAs comprises 3 to 20,000 of said CRISPR-Cas guide RNAs that hybridize to different target sequences within the genomic region of interest. For example, in some cases, the plurality of in vitro transcribed CRISPR-Cas guide RNAs comprises 10 to 100 (e.g., 20 to 90, 30 to 80, 40 to 70, 50 to 60) of said guide RNAs. In some cases, the plurality of in vitro transcribed CRISPR-Cas guide RNAs comprises 100 to 1000 (e.g., 200 to 900, 300 to 800, 400 to 700, 500 to 600) of said guide RNAs. In some cases, the plurality of in vitro transcribed CRISPR-Cas guide RNAs comprises 1000 to 10,000 (e.g., 2000 to 9000, 3000 to 8000, 4000 to 7000, 5000 to 6000) of said guide RNAs. In some cases, the plurality of in vitro transcribed CRISPR-Cas guide RNAs comprises 10,000 to 20,000 (e.g., 11,000 to 19,000, 12,000 to 18,000, 13,000 to 17,000, 14,000 to 16,000) of said guide RNAs.

In some embodiments, the guide RNAs target sequences selected from a subject list of chromatin accessible target sequences generated by the subject methods described herein. For example, the guide RNAs may be configured to comprise guide sequences that are complementary to target sequences selected from a subject list of chromatin accessible target sequences generated by the subject methods.

In some embodiments, the guide RNAs are single molecule guide RNAs such that (a), the guide sequence, and (b), the scaffold sequence, are present on the same nucleic acid molecule (e.g., as in a single guide RNA). In some embodiments, the guide RNAs are not single-molecule guide RNAs (e.g., are dual guide RNAs).

In some embodiments, the guide RNAs comprise a poly(A) tail, wherein the poly(A) tail of the guide RNA includes at least one adenosine that comprises a heterologous moiety. A poly(A) tail can be considered a nucleic acid sequence containing a plurality of repeating adenosine nucleotides and/or derivatives thereof. In some cases, the poly(A) tail may comprise two or more (e.g., 3 or more, 4 or more, 5 or more, 10 or more, 20 or more, 30 or more, 40 or more, 50 or more, 75 or more, or 100 or more) adenosine nucleotides, and/or derivatives thereof. For example, in some cases a poly(A) tail may be 2 to 100 nucleotides in length. For example, in some cases, the poly(A) tail may be 2 to 10 (e.g., 3 to 9, 4 to 8, 5 to 7) nucleotides in length. In some cases, the poly(A) tail may be 10 to 50 (e.g., 15 to 45, 20 to 40, 25 to 35) nucleotides in length. In some cases, the poly(A) tail may be 50 to 100 (e.g., 60 to 90, 70 to 80) nucleotides in length. In some cases, the poly(A) tail may be more than 100 nucleotides in length.

In some embodiments the poly(A) tails of the subject guide RNAs comprise at least one adenosine comprising a heterologous moiety. In some embodiments, 1%-100% of the adenosines in the poly(A) tail comprise a heterologous moiety. For example, in some cases 1%-10% (e.g., 2%-9%, 3%-8%, 4%-7%, 5%-6%) of the adenosines in the poly(A) tail comprise a heterologous moiety. For example, in some cases 10%-20% (e.g., 12%-19%, 13%-18%, 14%-17%, 15%-16%) of the adenosines in the poly(A) tail comprise a heterologous moiety. In some cases, 20%-30% (e.g., 22%-29%, 23%-28%, 24%-27%, 25%-26%) of the adenosines in the poly(A) tail comprise a heterologous moiety. In some cases, 30%-40% (e.g., 32%-39%, 33%-38%, 34%-37%, 35%-36%) of the adenosines in the poly(A) tail comprise a heterologous moiety. In some cases, 40%-50% (e.g., 42%-49%, 43%-48%, 44%-47%, 45%-46%) of the adenosines in the poly(A) tail comprise a heterologous moiety. In some cases, 50%-60% (e.g., 52%-59%, 53%-58%, 54%-57%, 55%-56%) of the adenosines in the poly(A) tail comprise a heterologous moiety. In some cases, 60%-70% (e.g., 62%-69%, 63%-68%, 64%-67%, 65%-66%) of the adenosines in the poly(A) tail comprise a heterologous moiety. In some cases, 70%-80% (e.g., 72%-79%, 73%-78%, 74%-77%, 75%-76%) of the adenosines in the poly(A) tail comprise a heterologous moiety. In some cases, 80%-90% (e.g., 82%-89%, 83%-88%, 84%-87%, 85%-86%) of the adenosines in the poly(A) tail comprise a heterologous moiety. In some cases, 90%-100% (e.g., 92%-99%, 93%-98%, 94%-97%, 95%-96%) of the adenosines in the poly(A) tail comprise a heterologous moiety.

In certain embodiments, when the guide RNAs are single molecule guide RNAs, the single molecule guide RNAs comprising a guide sequence (a) and a scaffold sequence (b) comprise a 3′ poly(A) tail. In certain cases, when the guide RNAs are not single molecule guide RNAs, the nucleic acid molecules comprising a guide sequence (a) comprise a 3′ poly(A) tail (e.g., crRNAs can include a 3′ poly(A) tail). In certain cases, when the guide RNAs are not single molecule guide RNAs, the nucleic acid molecule(s) that does not include the guide sequence (e.g., a tracrRNA) comprise a 3′ poly(A) tail. In some cases, when the guide RNAs are not single molecule guide RNAs, both the nucleic acid molecules (e.g., a crRNA and a tracrRNA) comprise a 3′ poly(A) tail.

In some embodiments, the guide RNAs comprise a heterologous moiety at their 5′ end (e.g., in some cases at the 5′ end of a sgRNA, in some cases at the 5′ end of a crRNA (of a dual guide RNA), in some cases at the 5′ end of a tracrRNA (of a dual guide RNA)). In certain embodiments, when the guide RNAs are single molecule guide RNAs, the single molecule guide RNAs comprising a guide sequence (a) and a scaffold sequence (b) comprise a heterologous moiety at their 5′ end. In certain cases, when the guide RNAs are not single molecule guide RNAs, the nucleic acid molecules comprising a guide sequence (a) comprise a heterologous moiety at their 5′ end (e.g., the crRNAs can include a heterologous moiety at their 5′ end). In certain cases, when the guide RNAs are not single molecule guide RNAs, the nucleic acid molecule(s) that do not include a guide sequence (e.g., a tracrRNA) comprise a heterologous moiety at their 5′ end. In some cases, when the guide RNAs are not single molecule guide RNAs, both the nucleic acid molecules comprise a heterologous moiety at their 5′ end.

In some embodiments, the guide RNAs comprise a heterologous moiety at their 5′ end and a 3′ poly(A) tail. In certain embodiments, when the guide RNAs are single molecule guide RNAs, the single molecule guide RNAs comprising a guide sequence (a) and a scaffold sequence (b) comprise both a heterologous moiety at their 5′ end and a 3′ poly(A) tail. In certain cases, when the guide RNAs are not single molecule guide RNAs, the nucleic acid molecules comprising a guide sequence (a) comprise a 3′ poly(A) tail and a heterologous moiety at their 5′ end (e.g., the crRNAs can include a heterologous moiety at their 5′ end and a 3′ poly(A) tail). In certain cases, when the guide RNAs are not single molecule guide RNAs, the nucleic acid molecules that do not include a guide sequence comprise a 3′ poly(A) tail and a heterologous moiety at their 5′ end (e.g., a tracrRNA can include a heterologous moiety at the 5′ end and a 3′ poly(A) tail). In certain cases, when the guide RNAs are not single molecule guide RNAs, one of the nucleic acid molecules can include a 5′ heterologous moiety and the other can include a 3′ poly(A) tail (e.g., the crRNA can include a 5′ heterogeneous moiety while the tracrRNA includes a 3′ poly(A) tail, or the crRNA can include a 3′ poly(A) tail while the tracrRNA includes a 5′ heterologous moiety).

In any given case, the heterologous moiety at the 5′ end of a guide RNA nucleic acid molecule may be different, and detectably distinguishable (e.g., as in the case for fluorescent dyes), from the one or more heterologous moieties of the 3′ poly(A) tail of guide RNA nucleic acid molecule. In some cases, when the guide RNAs are not single molecule guide RNAs, heterologous moieties comprised in the nucleic acid molecule(s) may be different, and detectably distinguishable (e.g., as in the case for fluorescent dyes), from one another.

CRISPR-CAS Guide RNAs with 3′ Poly(A) Tails

The present disclosure provides a CRISPR-Cas guide RNA comprising (a) a guide sequence that hybridizes to a target sequence; (b) a scaffold sequence that can bind to a CRISPR-Cas effector protein; and (c) a 3′ poly(A) tail, wherein at least one adenosine of the poly(A) tail comprises a heterologous moiety. In some embodiments, the guide RNAs are single molecule guide RNAs such that (a), the guide sequence, and (b), the scaffold sequence, are present on the same nucleic acid molecule (e.g., as in a single guide RNA). In some embodiments, the guide RNAs are not single-molecule guide RNAs (e.g., are dual guide, e.g., having a crRNA and a tracrRNA).

In some embodiments, the guide RNA comprises a poly(A) tail, wherein the poly(A) tail of the guide RNA includes at least one adenosine that comprises a heterologous moiety. A poly(A) tail can be considered a nucleic acid sequence containing a plurality of repeating adenosine nucleotides and/or derivatives thereof. In some cases, the poly(A) tail may comprise two or more (e.g., 3 or more, 4 or more, 5 or more, 10 or more, 20 or more, 30 or more, 40 or more, 50 or more, 75 or more, or 100 or more) adenosine nucleotides, and/or derivatives thereof. For example, in some cases a poly(A) tail may be 2 to 100 nucleotides in length. For example, in some cases, the poly(A) tail may be 2 to 10 (e.g., 3 to 9, 4 to 8, 5 to 7) nucleotides in length. In some cases, the poly(A) tail may be 10 to 50 (e.g., 15 to 45, 20 to 40, 25 to 35) nucleotides in length. In some cases, the poly(A) tail may be 50 to 100 (e.g., 60 to 90, 70 to 80) nucleotides in length. In some cases, the poly(A) tail may be more than 100 nucleotides in length.

In some embodiments the poly(A) tails of the subject guide RNAs comprise at least one adenosine comprising a heterologous moiety. In some embodiments, 1%-100% of the adenosines in the poly(A) tail comprise a heterologous moiety. For example, in some cases 1%-10% (e.g., 2%-9%, 3%-8%, 4%-7%, 5%-6%) of the adenosines in the poly(A) tail comprise a heterologous moiety. For example, in some cases 10%-20% (e.g., 12%-19%, 13%-18%, 14%-17%, 15%-16%) of the adenosines in the poly(A) tail comprise a heterologous moiety. In some cases, 20%-30% (e.g., 22%-29%, 23%-28%, 24%-27%, 25%-26%) of the adenosines in the poly(A) tail comprise a heterologous moiety. In some cases, 30%-40% (e.g., 32%-39%, 33%-38%, 34%-37%, 35%-36%) of the adenosines in the poly(A) tail comprise a heterologous moiety. In some cases, 40%-50% (e.g., 42%-49%, 43%-48%, 44%-47%, 45%-46%) of the adenosines in the poly(A) tail comprise a heterologous moiety. In some cases, 50%-60% (e.g., 52%-59%, 53%-58%, 54%-57%, 55%-56%) of the adenosines in the poly(A) tail comprise a heterologous moiety. In some cases, 60%-70% (e.g., 62%-69%, 63%-68%, 64%-67%, 65%-66%) of the adenosines in the poly(A) tail comprise a heterologous moiety. In some cases, 70%-80% (e.g., 72%-79%, 73%-78%, 74%-77%, 75%-76%) of the adenosines in the poly(A) tail comprise a heterologous moiety. In some cases, 80%-90% (e.g., 82%-89%, 83%-88%, 84%-87%, 85%-86%) of the adenosines in the poly(A) tail comprise a heterologous moiety. In some cases, 90%-100% (e.g., 92%-99%, 93%-98%, 94%-97%, 95%-96%) of the adenosines in the poly(A) tail comprise a heterologous moiety.

The heterologous moiety may be any heterologous moiety described herein. As an example, in certain embodiments the heterologous moiety is an azide group, such that the adenosine comprising the heterologous moiety is an azido-modified adenosine. In some cases, the heterologous moiety is a fluorescent dye, such as, e.g., any of the fluorescent dyes described herein.

In certain embodiments, when the guide RNA is a single molecule guide RNA, the single molecule guide RNA comprises a guide sequence (a) and a scaffold sequence (b) that comprise a 3′ poly(A) tail.

In some embodiments, the guide RNA comprises a heterologous moiety at its 5′ end and a 3′ poly(A) tail.

In any given case, the heterologous moiety at the 5′ end of a guide RNA nucleic acid molecule (e.g., 5′ end of a sgRNA, 5′ end of a crRNA, 5′ end of a tracrRNA) may be different, and detectably distinguishable (e.g., as in the case for fluorescent dyes), from the one or more heterologous moieties of the 3′ poly(A) tail of guide RNA nucleic acid molecule (e.g., 3′ end of a sgRNA, crRNA, tracrRNA).

In some embodiments, the guide RNAs target sequences selected from a subject list of chromatin accessible target sequences generated by the subject methods described herein. For example, the guide RNAs may be configured to comprise guide sequences that are complementary to target sequences selected from a subject list of chromatin accessible target sequences generated by the subject methods.

### Genomic Regions of Interest

In some embodiments, the plurality of in vitro transcribed CRISPR-Cas guide RNAs that hybridize to different target sequences within a genomic region of interest that is 500 to 2,000,000 contiguous nucleotides in length. For example, in some cases, the genomic region of interest is 500 to 5000 contiguous nucleotides (e.g., 1000 to 4500 contiguous nucleotides, 1500 to 4000 contiguous nucleotides, 2000 to 3500 contiguous nucleotides, 2500 to 3000 contiguous nucleotides) in length. In some cases, the genomic region of interest is 5000 to 20,000 contiguous nucleotides (e.g., 6000 to 19,000 contiguous nucleotides, 7000 to 18,000 contiguous nucleotides, 8000 to 17,000 contiguous nucleotides, 9000 to 16,000 contiguous nucleotides, 10,000 to 15,000 contiguous nucleotides, 11,000 to 14,000 contiguous nucleotides, 12,000 to 13,000 contiguous nucleotides) in length. In some cases, the genomic region of interest is 20,000 to 100,000 contiguous nucleotides (e.g., 30,000 to 90,000 contiguous nucleotides, 40,000 to 80,000 contiguous nucleotides, 50,000 to 70,000 contiguous nucleotides, 55,000 to 65,000 contiguous nucleotides) in length. In some cases, the genomic region of interest is 100,000 to 1,000,000 contiguous nucleotides (e.g., 200,000 to 900,000 contiguous nucleotides, 300,000 to 800,000 contiguous nucleotides, 400,000 to 700,000 contiguous nucleotides, 500,000 to 600,000 contiguous nucleotides) in length. In some cases, the genomic region of interest is 1,000,000 to 2,000,000 contiguous nucleotides (e.g., 1,100,000 to 1,900,000 contiguous nucleotides, 1,200,000 to 1,800,000 contiguous nucleotides, 1,300,000 to 1,700,000 contiguous nucleotides, 1,400,000 to 1,600,000 contiguous nucleotides) in length.

The genomic region of interest may a region in any genome of interest. In some embodiments, the genome is the genome of a eukaryotic cell (e.g., a protist, fungi, plant, and vertebrate eukaryotic cell). In some cases, the genome is a genome of a eukaryotic cell is a mammalian cell (e.g., a mammalian cell of humans; non-human primates; domestic and farm animals; and zoo, laboratory, sports, or pet animals, such as dogs, horses, cats, cows, mice, rats, rabbits, etc.). In some cases, the genome is a genome of a human cell.

In some embodiments, the genomic region of interest is a non-repetitive genomic region, as opposed to, e.g., a repetitive genomic region. In other words, a non-repetitive genomic region can be recognized as any genomic region that is not a repetitive genomic region.

Repetitive genomic regions are characterized by repeated patterns of nucleic acid sequences that occur, sometimes in tandem, in multiple copies throughout a genome. For example, repetitive genomic regions can include, without limitation, tandem repeats, microsatellites, minisatellites, DNA transposons (e.g., MITEs, Mavericks), and RNA transposons (e.g., LTRs, LINEs, SINEs). Repetitive genomic regions are known in the art and described, e.g., in Liao, Xingyu, et al. “Repetitive DNA sequence detection and its role in the human genome.” Communications Biology 6.1 (2023): 954; and Jurka, Jerzy, et al. “Repetitive sequences in complex genomes: structure and evolution.” Annu. Rev. Genomics Hum. Genet. 8.1 (2007): 241-259, the disclosures of which are incorporated by reference herein.

### Heterologous Moieties

As discussed above, the present disclosure provides a plurality of in vitro transcribed CRISPR-Cas guide RNAs that hybridize to different target sequences within a genomic region of interest. In some embodiments, the guide RNAs may comprise a poly(A) tail, wherein at least one adenosine of the poly(A) tail comprises a heterologous moiety. In some embodiments, the guide RNAs comprise a heterologous moiety at their 5′ end. The present disclosure also provides a CRISPR-Cas guide RNA comprising (a) a guide sequence that hybridizes to a target sequence; (b) a scaffold sequence that can bind to a CRISPR-Cas effector protein; and (c) a 3′ poly(A) tail, wherein at least one adenosine of the poly(A) tail comprises a heterologous moiety.

The heterologous moiety (e.g., of the 3′ poly(A) tail, and/or, e.g., at the 5′ end of the guide RNA, at the 5′ or 3′ end of a sgRNA, crRNA, tracrRNA, and the like) may be any desirable heterologous moiety that may facilitate, e.g., imaging, recruitment of other molecular species, stabilization of the molecule, or facilitate further modification of the guide RNA.

In some embodiments, the heterologous moiety may be a reactive heterologous moiety which may facilitate further modification of the guide RNA. Suitable reactive heterologous moieties may include, without limitation, azide and alkyne groups (e.g., cyclooctyne groups), vinyl groups, tetrazine, transcyclooctene, strained cyclooctynes (e.g., azadibenzylcyclooctyne; DBCO), dienes and dienophiles, and azide, sulfur (VI) fluoride exchange chemistry (SuFEX), sulfonyl fluoride, as well as hydroxyl, hydrazido, hydrazino, aldehyde, ketone, azido, alkyne, phosphine, epoxide, succinimide, and pentafluorophenyl (PFP) esters. Such heterologous moieties may be used to further modify the guide RNA by, e.g., conjugation of additional heterologous moieties via Click-chemistry.

In some embodiments, the heterologous moiety may be a fluorescent dye. Such a fluorescent dye may be advantageous, e.g., in imaging applications. Suitable examples of fluorescent dyes include, without limitation, Alexa Fluor dyes (e.g., A488, A565, A647, etc.), cyanine dyes (e.g., Cy3, Cy 5), Aloura dyes (e.g., Aloura 488, Aloura 594, etc.), ATTO dyes (e.g., ATTO 390, ATTO 425, ATTO 465) Brilliant dyes (e.g., Brilliant Violet 480), NovaFluor dyes (e.g., NovaFluor Blue 585, NovaFluor Red 685, NovaFluor Yellow 660, etc.) or any other suitable organic dye (e.g., coumarins, boron-dipyrromethenes, fluoresceins, rhodamines, etc.), and derivatives thereof. For example, derivatives of a suitable fluorescent dye can include derivatives comprising a reactive moiety. Such reactive moieties can include, without limitation, azide and alkyne groups (e.g., cyclooctyne groups), vinyl groups, tetrazine, transcyclooctene, strained cyclooctynes (e.g., azadibenzylcyclooctyne; DBCO), dienes and dienophiles, and azide, sulfur (VI) fluoride exchange chemistry (SuFEX), sulfonyl fluoride, as well as hydroxyl, hydrazido, hydrazino, aldehyde, ketone, azido, alkyne, phosphine, epoxide, succinimide, and pentafluorophenyl (PFP) esters.

As would be appreciated by the skilled person in the art, the particular fluorescent dye may be chosen based on the desired application of the subject guide RNAs.

### CRISPR-Cas Effector Proteins

As discussed above, the present disclosure provides a plurality of in vitro transcribed CRISPR-Cas guide RNAs, each guide RNA comprising: (a) a guide sequence that hybridizes to a target sequence; and (b) a scaffold sequence that can bind to a CRISPR-Cas effector protein, that hybridize to different target sequences within a genomic region of interest. The present disclosure also provides a CRISPR-Cas guide RNA comprising (a) a guide sequence that hybridizes to a target sequence; (b) a scaffold sequence that can bind to a CRISPR-Cas effector protein; and (c) a 3′ poly(A) tail, wherein at least one adenosine of the poly(A) tail comprises a heterologous moiety.

Examples of CRISPR-Cas effector proteins (also referred to as “CRISPR-Cas effector polypeptides” herein) are CRISPR-Cas endonucleases (e.g., class 2 CRISPR-Cas effector polypeptide such as a type II, or type V, CRISPR-Cas effector polypeptide). Where a CRISPR-Cas effector polypeptide has endonuclease activity, the CRISPR-Cas effector polypeptide may also be referred to as a “CRISPR-Cas endonuclease.” A CRISPR-Cas effector polypeptide can also have reduced or undetectable endonuclease activity. A CRISPR-Cas effector polypeptide can also be a fusion CRISPR-Cas effector polypeptide comprising a heterologous fusion partner. In some cases, a suitable CRISPR-Cas effector polypeptide is a class 2 CRISPR-Cas effector polypeptide. In some cases, a suitable CRISPR-Cas effector polypeptide is a class 2 type II CRISPR-Cas effector polypeptide (e.g., a Cas9 protein). In some cases, a suitable CRISPR-Cas effector polypeptide is a class 2 type V CRISPR-Cas endonuclease (e.g., a Cpf1 protein, a C2c1 protein, or a C2c3 protein).

In class 2 CRISPR systems, the functions of the effector complex (e.g., the cleavage of target DNA) are carried out by a single endonuclease (e.g., see Zetsche et al., Cell. 2015 Oct. 22; 163 (3): 759-71; Makarova et al., Nat Rev Microbiol. 2015 November; 13 (11): 722-36; Shmakov et al., Mol Cell. 2015 Nov. 5; 60 (3): 385-97); and Shmakov et al. (2017) Nature Reviews Microbiology 15:169. As such, the term “class 2 CRISPR-Cas protein” is used herein to encompass the CRISPR-Cas effector polypeptide (e.g., the target nucleic acid cleaving protein) from class 2 CRISPR systems. Thus, the term “class 2 CRISPR-Cas effector polypeptide” as used herein encompasses type II CRISPR-Cas effector polypeptides (e.g., Cas9); type V-A CRISPR-Cas effector polypeptides (e.g., Cpf1 (also referred to a “Cas12a”)); type V-B CRISPR-Cas effector polypeptides (e.g., C2c1 (also referred to as “Cas12b”)); type V-C CRISPR-Cas effector polypeptides (e.g., C2c3 (also referred to as “Cas12c”)); type V-U1 CRISPR-Cas effector polypeptides (e.g., C2c4); type V-U2 CRISPR-Cas effector polypeptides (e.g., C2c8); type V-U5 CRISPR-Cas effector polypeptides (e.g., C2c5); type V-U4 CRISPR-Cas proteins (e.g., C2c9); type V-U3 CRISPR-Cas effector polypeptides (e.g., C2c10). To date, class 2 CRISPR-Cas effector polypeptides encompass type II, type V, and type VI CRISPR-Cas effector polypeptides, but the term is also meant to encompass any class 2 CRISPR-Cas effector polypeptide suitable for binding to a corresponding guide RNA and forming an RNP complex.

### CRISPR-CAS Guide RNA Template Nucleic Acids

The present disclosure provides a plurality of guide RNA template nucleic acids, each template nucleic acid comprising: (a) a first primer binding domain common to a subset of the plurality of template nucleic acids; (b) an RNA-polymerase promoter; and (c) a sequence encoding a guide RNA, wherein the encoded guide RNA comprises: (i) a guide sequence that hybridizes to a target sequence; and (ii) a sequence common to the plurality of template nucleic acids, wherein the guide sequences encoded in the template nucleic acids hybridize to target sequences within a genomic region of interest at least 500 (e.g., at least 1000, at least 2000, at least 3000, at least 4000, at least 5000, at least 10,000, at least 20,000, at least 30,000, at least 40,000, at least 50,000, at least 100,000, at least at least 200,000, at least at least 300,000, at least at least 400,000, at least at least 500,000, at least 1,000,000, at least 1,000,000, at least 1,250,000, at least 1,500,000, at least 1,750,000, or at least 2,000,000) contiguous nucleotides in length.

In some cases, e.g., for dual guide RNAs, the two nucleic acids (crRNA and tracrRNA) can be separately transcribed from respective template nucleic acids (e.g., by in vitro transcription) and later complexed to form a guide RNA. In some cases, one of the nucleic acids (e.g., the tracrRNA) is chemically synthesized while the crRNA is in vitro transcribed. In such cases, the guide RNA would still be considered herein to have been in vitro transcribed. In some embodiments, the sequence common to the plurality of template nucleic acids of (c) (ii) comprises a scaffold sequence that can bind to a CRISPR-Cas effector protein. In such cases, the encoded guide RNA may include a targeting and protein-binding segment, as in the case of a single guide RNA.

In some embodiments the plurality of template nucleic acids comprises 3 or more (e.g., 5 or more, 10 or more, 20 or more, 50 or more, 100 or more, 250 or more, 500 or more, 1000 or more, 2000 or more, 5000 or more, 10,000 or more, 15,000 or more, 20,000 or more) of said template nucleic acids. In some cases, the plurality of template nucleic acids comprises 3 to 20,000 of said template nucleic acids. For example, in some cases the plurality of template nucleic acids comprises 10 to 100 (e.g., 20 to 90, 30 to 80, 40 to 70, 50 to 60) of said template nucleic acids. In some cases, the plurality template nucleic acids comprises 100 to 1000 (e.g., 200 to 900, 300 to 800, 400 to 700, 500 to 600) of said template nucleic acids. In some cases, the plurality of template nucleic acids comprises 1000 to 10,000 (e.g., 2000 to 9000, 3000 to 8000, 4000 to 7000, 5000 to 6000) of said template nucleic acids. In some cases, the plurality of template nucleic acids comprises 10,000 to 20,000 (e.g., 11,000 to 19,000, 12,000 to 18,000, 13,000 to 17,000, 14,000 to 16,000) of said template nucleic acids.

In some embodiments, the guide sequences encoded in the template nucleic acids hybridize to a target sequence within a genomic region of interest 500 to 2,000,000 contiguous nucleotides in length. For example, in some cases, the genomic region of interest is 500 to 5000 contiguous nucleotides (e.g., 1000 to 4500 contiguous nucleotides, 1500 to 4000 contiguous nucleotides, 2000 to 3500 contiguous nucleotides, 2500 to 3000 contiguous nucleotides) in length. In some cases, the genomic region of interest is 5000 to 20,000 contiguous nucleotides (e.g., 6000 to 19,000 contiguous nucleotides, 7000 to 18,000 contiguous nucleotides, 8000 to 17,000 contiguous nucleotides, 9000 to 16,000 contiguous nucleotides, 10,000 to 15,000 contiguous nucleotides, 11,000 to 14,000 contiguous nucleotides, 12,000 to 13,000 contiguous nucleotides) in length. In some cases, the genomic region of interest is 20,000 to 100,000 contiguous nucleotides (e.g., 30,000 to 90,000 contiguous nucleotides, 40,000 to 80,000 contiguous nucleotides, 50,000 to 70,000 contiguous nucleotides, 55,000 to 65,000 contiguous nucleotides) in length. In some cases, the genomic region of interest is 100,000 to 1,000,000 contiguous nucleotides (e.g., 200,000 to 900,000 contiguous nucleotides, 300,000 to 800,000 contiguous nucleotides, 400,000 to 700,000 contiguous nucleotides, 500,000 to 600,000 contiguous nucleotides) in length. In some cases, the genomic region of interest is 1,000,000 to 2,000,000 contiguous nucleotides (e.g., 1,100,000 to 1,900,000 contiguous nucleotides, 1,200,000 to 1,800,000 contiguous nucleotides, 1,300,000 to 1,700,000 contiguous nucleotides, 1,400,000 to 1,600,000 contiguous nucleotides) in length.

In some embodiments, the guide RNAs encoded in the subject template nucleic acids target (i.e., are targeted to) target sequences selected from a subject list of chromatin accessible target sequences generated according to the methods described herein.

Each subject template nucleic acid may be, or maybe present on, without limitation: a circular nucleic acid (e.g., a plasmid), a linear nucleic acid or oligonucleotide, a double-stranded nucleic acid (e.g., dsDNA oligonucleotide), a single-stranded nucleic acid (e.g., a ssDNA oligonucleotide). In some cases, the subject template nucleic acids may be in solution (e.g., as a pooled plurality of template nucleic acids.).

The first primer binding domain that is common to a subset of the plurality of template nucleic acids may be useful in selectively amplifying subsets of the plurality for template nucleic acids for further use. In some embodiments, the first primer binding domain that is common to one of at least two subsets. In some cases, the first primer binding domain that is common to one of at 2-10 (e.g., 3-7, 4-6, 5) subsets of the plurality of template nucleic acids. In some cases, the first primer binding domain that is common to one of 10-100 (e.g., 20-90, 30-80, 40-70, 50-60) subsets of the plurality of template nucleic acids.

The RNA polymerase promoter may be any suitable RNA polymerase promoter known in the art (e.g., any RNA polymerase promoter for a corresponding RNA polymerase that is suitable for in vitro transcription). Exemplary RNA polymerase promoters include, without limitation, T7 RNA polymerase promoters, and SP6 RNA polymerase promoters. In some cases, a T7 RNA polymerase promoter may include the sequence TAATACGACTCACTATAG (SEQ ID NO: 17). In some cases, a SP6 RNA polymerase promoter may include the sequence ATTTAGGTGACACTATAG (SEQ ID NO: 18).

The RNA polymerase promoter will be positioned within the nucleic acid such that the promoter is operably linked to the sequence encoding the guide RNA (or, e.g., to the sequence encoding the portions of the guide RNA, e.g., crRNA, tracrRNA). As is known in the art, the positioning of the RNA polymerase promoter relative to an operably linked sequence may vary across different promoters. In some embodiments, the subject template nucleic acids may comprise an RNA polymerase promoter immediately 5′ of the sequence encoding a guide RNA.

As discussed, the RNA polymerase promoter may be any RNA polymerase promoter suitable for in vitro transcription. In vitro transcription methods are known in the art and described, e.g., in Shen, Yuan, et al. “A review of advances in in vitro RNA preparation by ssRNAP.” International Journal of Biological Macromolecules (2025): 141002, the disclosure of which is incorporated by reference herein.

### Methods for Labeling CRISPR-Cas Guide RNAs

The present disclosure provides methods for labeling a CRISPR-Cas guide RNA, the method comprising: contacting a CRISPR-Cas guide RNA (or, e.g., a crRNA and/or tracrRNA in the case of a dual guide RNA) with a poly(A) polymerase and adenosine triphosphate (ATP) molecules, wherein at least one ATP molecule comprises a heterologous moiety, thereby generating a CRISPR-Cas guide RNA comprising a poly(A) tail in which at least one adenosine of the poly(A) tail comprises the heterologous moiety.

The poly(A) polymerase may be any poly(A) polymerase suitable for in vitro poly(A) tailing of RNAs. In some embodiments, the poly(A) polymerase may be a recombinant E. coli poly(A) polymerase or a recombinant yeast (e.g., Saccharomyces cerevisae). Recombinant poly(A) polymerases, such as those derived from E. coli or Yeast are readily commercially available. The contacting of the CRISPR-Cas guide RNA with a poly(A) polymerase and adenosine triphosphate (ATP) molecules, may be carried out under suitable conditions for poly(A) tailing known in the art and described, e.g., in Boseley, P. G., T. Moss, and M. L. Birnstiel. “[55] 5′ labeling and poly(A) tailing.” Methods in enzymology. Vol. 65. Academic Press, 1980. 478-494, the disclosure of which is incorporated by reference herein.

In some embodiments, the ATP molecules comprise a mixture of ATP molecules that comprise a heterologous moiety and ATP molecules that do not comprise a heterologous moiety. In some cases, all the ATP molecules may comprise a heterologous moiety. In the subject methods, contacting a CRISPR-Cas guide RNA with a poly(A) polymerase and adenosine triphosphate (ATP) molecules results in addition of the ATP molecules to the 3′ end of the guide RNA nucleic acid, thereby generating a CRISPR-Cas guide RNA comprising a poly(A) tail in which at least one adenosine of the poly(A) tail comprises the heterologous moiety.

In cases where the ATP molecules comprise a mixture of ATP molecules comprising a heterologous moiety and ATP molecules not comprising a heterologous moiety, the ratio of the two species may be adjusted to control the relative percentage of adenosines comprising a heterologous moiety that are incorporated into the poly(A) tail. By way of example, a mixture wherein 50% of the ATP molecules comprise a heterologous moiety may be utilized when it is desired to produce a poly(A) tail wherein approximately 50% of the adenosine nucleotides in said poly(A) tail comprise a heterologous moiety. In some cases, all the ATP molecules may comprise a heterologous moiety such that the methods result in a poly(A) tail where 100% of the adenosines comprise a heterologous moiety.

The parameters under which the CRISPR-Cas guide RNA is contacted with a poly(A) polymerase and adenosine triphosphate (ATP) molecules (e.g., concentration of the reactants, concentration of the poly(A) polymerase, temperature of the reaction, length of time for said contacting) may be adjusted to vary the average length of the poly(A) tails generated in the subject methods. In some embodiments, the poly(A) tail generated by the subject methods may comprise two or more (e.g., 3 or more, 4 or more, 5 or more, 10 or more, 20 or more, 30 or more, 40 or more, 50 or more, 75 or more, or 100 or more) adenosine nucleotides, and/or derivatives thereof. For example, in some cases a poly(A) tail may be 2 to 100 nucleotides in length. For example, in some cases, the poly(A) tail may be 2 to 10 (e.g., 3 to 9, 4 to 8, 5 to 7) nucleotides in length. In some cases, the poly(A) tail may be 10 to 50 (e.g., 15 to 45, 20 to 40, 25 to 35) nucleotides in length. In some cases, the poly(A) tail may be 50 to 100 (e.g., 60 to 90, 70 to 80) nucleotides in length. In some cases, the poly(A) tail may be more than 100 nucleotides in length.

In some embodiments the poly(A) tail generated by the subject methods comprises at least one adenosine comprising a heterologous moiety. In some embodiments, 1%-100% of the adenosines in the poly(A) tail comprise a heterologous moiety. For example, in some cases 1%-10% (e.g., 2%-9%, 3%-8%, 4%-7%, 5%-6%) of the adenosines in the poly(A) tail comprise a heterologous moiety. For example, in some cases 10%-20% (e.g., 12%-19%, 13%-18%, 14%-17%, 15%-16%) of the adenosines in the poly(A) tail comprise a heterologous moiety. In some cases, 20%-30% (e.g., 22%-29%, 23%-28%, 24%-27%, 25%-26%) of the adenosines in the poly(A) tail comprise a heterologous moiety. In some cases, 30%-40% (e.g., 32%-39%, 33%-38%, 34%-37%, 35%-36%) of the adenosines in the poly(A) tail comprise a heterologous moiety. In some cases, 40%-50% (e.g., 42%-49%, 43%-48%, 44%-47%, 45%-46%) of the adenosines in the poly(A) tail comprise a heterologous moiety. In some cases, 50%-60% (e.g., 52%-59%, 53%-58%, 54%-57%, 55%-56%) of the adenosines in the poly(A) tail comprise a heterologous moiety. In some cases, 60%-70% (e.g., 62%-69%, 63%-68%, 64%-67%, 65%-66%) of the adenosines in the poly(A) tail comprise a heterologous moiety. In some cases, 70%-80% (e.g., 72%-79%, 73%-78%, 74%-77%, 75%-76%) of the adenosines in the poly(A) tail comprise a heterologous moiety. In some cases, 80%-90% (e.g., 82%-89%, 83%-88%, 84%-87%, 85%-86%) of the adenosines in the poly(A) tail comprise a heterologous moiety. In some cases, 90%-100% (e.g., 92%-99%, 93%-98%, 94%-97%, 95%-96%) of the adenosines in the poly(A) tail comprise a heterologous moiety.

In some embodiments, the heterologous moiety may be a reactive moiety that may facilitate further modification of the adenosines once incorporated into the poly(A) tail. For example, in some cases the reactive heterologous moiety suitable for Click-chemistry reactions. In some embodiments, the ATP molecules a heterologous moiety including, without limitation, azide and alkyne groups (e.g., cyclooctyne groups), vinyl groups, tetrazine, transcyclooctene, strained cyclooctynes (e.g., azadibenzylcyclooctyne; DBCO), dienes and dienophiles, and azide, sulfur (VI) fluoride exchange chemistry (SuFEX), sulfonyl fluoride, as well as hydroxyl, hydrazido, hydrazino, aldehyde, ketone, azido, alkyne, phosphine, epoxide, succinimide, and pentafluorophenyl (PFP) esters. In some cases, the ATP molecules comprising a heterologous moiety are ribonucleic acid derivatives. In some cases, the ATP molecules comprising a heterologous moiety are deoxyribonucleic acid derivatives (i.e., dATP derivatives). In a particular embodiment, the heterologous moiety is an azide moiety such that the ATP molecules a heterologous moiety are 2′-Azido-2′-dATP molecules.

In some embodiments, the subject methods for labeling a guide RNA further comprise conjugating a fluorescent dye to a heterologous moiety of an adenosine in the poly(A) tail. Suitable examples of fluorescent dyes include, without limitation, Alexa Fluor dyes (e.g., A488, A565, A647, etc.), cyanine dyes (e.g., Cy3, Cy 5), Aloura dyes (e.g., Aloura 488, Aloura 594, etc.), ATTO dyes (e.g., ATTO 390, ATTO 425, ATTO 465) Brilliant dyes (e.g., Brilliant Violet 480), NovaFluor dyes (e.g., NovaFluor Blue 585, NovaFluor Red 685, NovaFluor Yellow 660, etc.) or any other suitable organic dye (e.g., coumarins, boron-dipyrromethenes, fluoresceins, rhodamines, etc.), and derivatives thereof. For example, derivatives of a suitable fluorescent dye can include derivatives comprising a reactive moiety (e.g., a reactive moiety suitable for Click-chemistry). Such reactive moieties can include, without limitation, azide and alkyne groups (e.g., cyclooctyne groups), vinyl groups, tetrazine, transcyclooctene, strained cyclooctynes (e.g., azadibenzylcyclooctyne; DBCO), dienes and dienophiles, and azide, sulfur (VI) fluoride exchange chemistry (SuFEX), sulfonyl fluoride, as well as hydroxyl, hydrazido, hydrazino, aldehyde, ketone, azido, alkyne, phosphine, epoxide, succinimide, and pentafluorophenyl (PFP) esters. In a particular embodiment, the fluorescent dye comprises an azadibenzylcyclooctyne (DBCO) moiety. In some embodiments the conjugation step is performed via Click-chemistry.

Click-chemistry reactions that may be employed include (i) nucleophilic substitutions; (ii) additions to C-C multiple bonds (e.g., Michael addition, epoxidation, dihydroxylation, aziridination); (iii) nonaldol like chemistry (e.g., N-hydroxysuccinimide active ester couplings); and (iv) cycloadditions (e.g., Diels-Adler reaction, Huisgen's cycloaddition). Huisgen's cycloaddition has been applied in various branches of chemistry. It consists of the condensation of organic azides with alkyne groups to form 1,2,3-triazole linkages. Azide and alkyne functionalities can be easily introduced in the scaffold of large organic constructs of biological relevance. The reaction may be catalyzed by introducing copper (I). The Cu (I) core has a dual effect in that it activates the slow-reacting alkyne group thus accelerating the azide-alkyne condensation kinetics by ~107-108-fold, and it organizes the reacting groups by “templation” so that only a regiospecific 1,4-disubstituted adduct is formed. This reaction is known as the copper-catalyzed azide alkyne cycloaddition (CuAAC), and its compatibility with a wide range of biological substrates and synthetic conditions makes CuAAC the flagship among click conjugations. Since its discovery, Cu (I)-catalyzed azide alkyne cycloaddition has been widely used within the fields of biology, biochemistry, and biotechnology. Click-chemistry reactions that may be employed to attach the nucleotide or polynucleotide to the particle include, but are not limited to, Huisgen Azide-Alkyne 1,3-Dipolar Cycloaddition, Copper-Catalyzed Azide-Alkyne Cycloaddition (CuAAC), Ruthenium-Catalyzed Azide-Alkyne Cycloaddition (RuAAC), and the like. Details regarding click-chemistry with nucleic acids are found, e.g., in Fantoni et al. (2021) Chem. Rev. 121 (12): 7122-7154.

In some cases, the reaction is a copper-free click chemistry. Copper-free click chemistry is an alternative approach to click chemistry that proceeds at a lower activation barrier and is free of cytotoxic transition metal catalysts. The copper-free reaction can use ring strain in place of the copper catalyst to promote a [3+2] azide-alkyne cycloaddition reaction. For example, 8-carbon closed ring structure of a cyclooctyne comprising an internal alkyne bond induces a substantial bond angle deformation of the acetylene, which is highly reactive with azide groups to form a triazole. Thus, cyclooctyne derivatives may be used for copper-free click reactions, without the toxic copper catalyst. Another type of copper-free click reaction was reported by Ning et al. (2010, Angew Chem Int Ed 49:3065-68), involving strain-promoted alkyne-nitron cycloaddition. To address the slow rate of the original cyclooctyne reaction, electron-withdrawing groups can be attached adjacent to the triple bond. Examples of such substituted cyclooctynes include difluorinated cyclooctynes, 4-dibenzocyclooctynol and azacyclooctyne. An alternative copper-free reaction involved strain-promoted alkyne-nitrone cycloaddition to give N-alkylated isoxazolines.

Accordingly, in some embodiments the copper-free click chemistry useful in conjugating a fluorescent dye to a heterologous moiety of an adenosine in the subject methods can be selected from: (a) strain-promoted azide/dibenzocyclooctyne-amine (DBCO) click chemistry; (b) inverse electron demand Diels-Alder (IED-DA) tetrazine/trans-cyclooctene (TCO) click chemistry; (c) inverse electron demand Diels-Alder (IED-DA) tetrazine/norbonene click chemistry; (d) Diels-Alder maleimide/furan click-chemistry; (e) Staudinger ligation; and (f) nitrile-oxide/norbonene cycloaddition click chemistry.

Methods for applying Click-Chemistry to nucleic acids that are suitable for use in the subject labeling methods disclosed herein are known in the art and described, e.g., in Fantoni, Nicolò Zuin, Afaf H. El-Sagheer, and Tom Brown. “A hitchhiker's guide to click-chemistry with nucleic acids.” Chemical Reviews 121.12 (2021): 7122-7154, the disclosure of which is incorporated by reference herein.

### Methods of Preparing a Plurality of CRISPR-Cas Guide RNAs

The present disclosure provides methods for preparing a plurality of CRISPR-Cas guide RNAs for targeting a contiguous genomic region, the method comprising: (a) contacting a plurality of template nucleic acids, or a subset thereof, with an RNA polymerase; and (b) transcribing the plurality of template nucleic acids, or a subset thereof, to produce a plurality of in vitro transcribed CRISPR-Cas guide RNAs for targeting a contiguous genomic region. In some embodiments, the plurality of template nucleic acids may be any subject plurality of template nucleic acids described herein.

The RNA polymerase may be any suitable RNA polymerase known in the art (e.g., any RNA polymerase suitable for in vitro transcription). Exemplary RNA polymerases suitable for the subject methods include T7 RNA polymerase and SP6 RNA polymerase. Recombinant T7 and SP6 RNA polymerases are known in the art readily commercially available. In addition, the in vitro transcription may be carried out under any suitable conditions known in the art. In vitro transcription methods are known in the art and described, e.g., in Shen, Yuan, et al. “A review of advances in in vitro RNA preparation by ssRNAP.” International Journal of Biological Macromolecules (2025): 141002, the disclosure of which is incorporated by reference herein.

In some embodiments, the methods further comprise, prior to said contacting of (a) contacting the template nucleic acid composition with a forward primer and a reverse primer to amplify the plurality of template nucleic acids, or a subset thereof. In some cases, the method comprises amplifying a subset of the plurality of template nucleic acids. For example, in some cases, the methods comprise amplifying one of at least two subsets. In some cases, the methods comprise amplifying one of 2-10 (e.g., 3-7, 4-6, 5) subsets. In some cases, the methods comprise amplifying one of 10-100 (e.g., 20-90, 30-80, 40-70, 50-60) subsets.

In some cases, the first primer is complementary to a first primer binding domain in all or a subset of the template nucleic acids. In some embodiments, the first primer is complementary to a first primer binding domain common to one of at least two subsets. In some cases, the first primer is complementary to a first primer binding domain that is common to one of 2-10 (e.g., 3-7, 4-6, 5) subsets of the plurality of template nucleic acids. In some cases, the first primer is complementary to a first primer binding domain that is common to one of 10-100 (e.g., 20-90, 30-80, 40-70, 50-60) subsets of the plurality of template nucleic acids. In some embodiments, the reverse primer is complementary to a sequence common to the plurality of template nucleic acids (e.g., a scaffold sequence of the encoded guide RNA). Methods for designing primers and primer binding domains are known in the art, e.g., the Primer designing tool available at ncbi(.)nlm(.)nih(.)gov/tools/primer-blast/.

In some embodiments, the subject methods further comprise, after said transcribing step of (b), labeling the plurality of in vitro transcribed CRISPR-Cas guide RNAs. In some embodiments, the subject guide RNAs may be labeled by any of the subject labeling methods disclosed herein.

In some embodiments, the subject methods further comprises, after said transcribing step of (b) contacting the plurality of in vitro transcribed CRISPR-Cas guide RNAs with a CRISPR-Cas effector protein to produce a plurality of CRISP-Cas effector complexes. In varying embodiments, the CRISPR-Cas effector protein may be any CRISPR-Cas effector protein described herein. For example, in some cases the Class 2 CRISPR-Cas effector protein. In some cases, the Class 2 CRISPR-Cas effector protein is a Type II or Type V CRISPR-Cas effector protein as described herein.

### Methods for Guiding CRISPR-Cas Effector Complexes to a Contiguous Genomic Region of Interest

The present disclosure provides methods for guiding a plurality of CRISPR-Cas effector complexes to a contiguous genomic region of interest, the method comprising contacting a contiguous genomic region in a target cell with a plurality of CRISPR-Cas effector complexes, wherein each CRISPR-Cas effector complex in the plurality of complexes comprises: (a) a CRISPR-Cas effector protein; and (b) an in vitro transcribed guide RNA that hybridizes to a target sequence within the contiguous genomic region, wherein the plurality comprises 3 or more (e.g., 5 or more, 10 or more, 20 or more, 50 or more, 100 or more, 250 or more, 500 or more, 1000 or more, 2000 or more, 5000 or more, 10,000 or more, 15,000 or more, 20,000 or more) of said guide RNAs, and wherein the genomic region of interest is at least 500 (e.g., at least 1000, at least 2000, at least 3000, at least 4000, at least 5000, at least 10,000, at least 20,000, at least 30,000, at least 40,000, at least 50,000, at least 100,000, at least at least 200,000, at least at least 300,000, at least at least 400,000, at least at least 500,000, at least 1,000,000, at least 1,000,000, at least 1,250,000, at least 1,500,000, at least 1,750,000, or at least 2,000,000) contiguous nucleotides in length, thereby guiding the CRISPR-Cas effector complex to the contiguous genomic region.

In some embodiments, the methods comprise contacting a contiguous genomic region in a target cell with a plurality of CRISPR-Cas effector complexes, wherein said plurality comprises 3 to 20,000 of said in vitro transcribed guide RNAs and the contiguous genomic region is 500 to 2,000,000 contiguous nucleotides in length.

For example, in some cases, the plurality of CRISPR-Cas effector complexes comprises 10 to 100 (e.g., 20 to 90, 30 to 80, 40 to 70, 50 to 60) of said guide RNAs. In some cases, the plurality of CRISPR-Cas effector complexes comprises 100 to 1000 (e.g., 200 to 900, 300 to 800, 400 to 700, 500 to 600) of said guide RNAs. In some cases, the plurality of CRISPR-Cas effector complexes comprises 1000 to 10,000 (e.g., 2000 to 9000, 3000 to 8000, 4000 to 7000, 5000 to 6000) of said guide RNAs. In some cases, the plurality of CRISPR-Cas effector complexes comprises 10,000 to 20,000 (e.g., 11,000 to 19,000, 12,000 to 18,000, 13,000 to 17,000, 14,000 to 16,000) of said guide RNAs.

In some cases, the genomic region of interest is 500 to 5000 contiguous nucleotides (e.g., 1000 to 4500 contiguous nucleotides, 1500 to 4000 contiguous nucleotides, 2000 to 3500 contiguous nucleotides, 2500 to 3000 contiguous nucleotides) in length. In some cases, the genomic region of interest is 5000 to 20,000 contiguous nucleotides (e.g., 6000 to 19,000 contiguous nucleotides, 7000 to 18,000 contiguous nucleotides, 8000 to 17,000 contiguous nucleotides, 9000 to 16,000 contiguous nucleotides, 10,000 to 15,000 contiguous nucleotides, 11,000 to 14,000 contiguous nucleotides, 12,000 to 13,000 contiguous nucleotides) in length. In some cases, the genomic region of interest is 20,000 to 100,000 contiguous nucleotides (e.g., 30,000 to 90,000 contiguous nucleotides, 40,000 to 80,000 contiguous nucleotides, 50,000 to 70,000 contiguous nucleotides, 55,000 to 65,000 contiguous nucleotides) in length. In some cases, the genomic region of interest is 100,000 to 1,000,000 contiguous nucleotides (e.g., 200,000 to 900,000 contiguous nucleotides, 300,000 to 800,000 contiguous nucleotides, 400,000 to 700,000 contiguous nucleotides, 500,000 to 600,000 contiguous nucleotides) in length. In some cases, the genomic region of interest is 1,000,000 to 2,000,000 contiguous nucleotides (e.g., 1,100,000 to 1,900,000 contiguous nucleotides, 1,200,000 to 1,800,000 contiguous nucleotides, 1,300,000 to 1,700,000 contiguous nucleotides, 1,400,000 to 1,600,000 contiguous nucleotides) in length.

In the subject methods, the number of in vitro transcribed guide RNAs in the plurality of CRISPR-Cas effector complexes may vary according to the length of the contiguous genomic region of interest. In some embodiments, the number of guide RNAs in the plurality of CRISPR-Cas effector complexes may be chosen such that there is approximately one guide RNA for every 100 base pairs (bp)-150 base pairs (bp) in the genomic region of interest. In other words, the number of said guide RNAs may be chosen such that said guide RNAs may hybridize to target a sequence that occurs, on average, every 100 bp-150 bp in the genomic region of interest. In some embodiments, the number of guide RNAs in the plurality of CRISPR-Cas effector complexes may be chosen such that there is approximately one guide RNA for every 100 bp-110 bp (e.g., 101 bp-109 bp, 102 bp-108 bp, 103 bp-107 bp, 104 bp-106 bp) in the genomic region of interest. In some embodiments, the number of guide RNAs in the plurality of CRISPR-Cas effector complexes may be chosen such that there is approximately one guide RNA for every 110 bp-120 bp (e.g., 111 bp-119 bp, 112 bp-118 bp, 113 bp-117 bp, 114 bp-116 bp) in the genomic region of interest. In some embodiments, the number of guide RNAs in the plurality of CRISPR-Cas effector complexes may be chosen such that there is approximately one guide RNA for every 120 bp-130 bp (e.g., 121 bp-129 bp, 122 bp-128 bp, 123 bp-127 bp, 124 bp-126 bp) in the genomic region of interest. In some embodiments, the number of guide RNAs in the plurality of CRISPR-Cas effector complexes may be chosen such that there is approximately one guide RNA for every 130 bp-140 bp (e.g., 131 bp-139 bp, 132 bp-138 bp, 133 bp-137 bp, 134 bp-136 bp) in the genomic region of interest. In some embodiments, the number of guide RNAs in the plurality of CRISPR-Cas effector complexes may be chosen such that there is approximately one guide RNA for every 140 bp-150 bp (e.g., 141 bp-149 bp, 142 bp-148 bp, 143 bp-147 bp, 144 bp-146 bp) in the genomic region of interest.

In some embodiments, the contiguous genomic region of interest is a non-repetitive genomic region, as opposed to, e.g., a repetitive genomic region. In other words, a non-repetitive genomic region can be recognized as any genomic region that is not a repetitive genomic region.

Repetitive genomic regions are characterized by repeated patterns of nucleic acid sequences that occur, sometimes in tandem, in multiple copies throughout a genome. For example, repetitive genomic regions can include, without limitation, tandem repeats, microsatellites, minisatellites, DNA transposons (e.g., MITEs, Mavericks), and RNA transposons (e.g., LTRs, LINEs, SINEs). Repetitive genomic regions are known in the art and described, e.g., in Liao, Xingyu, et al. “Repetitive DNA sequence detection and its role in the human genome.” Communications Biology 6.1 (2023): 954; and Jurka, Jerzy, et al. “Repetitive sequences in complex genomes: structure and evolution.” Annu. Rev. Genomics Hum. Genet. 8.1 (2007): 241-259, the disclosures of which are incorporated by reference herein.

In certain embodiments, the in vitro transcribed guide RNAs of the plurality of CRISPR-Cas effector complexes may be any of the subject plurality of in vitro transcribed guide RNAs described herein. For example, in some embodiments, the in vitro transcribed guide RNAs of the plurality of CRISPR-Cas effector complexes may be specific for a target sequence selected from a list of chromatin accessible guide RNAs generated by the subject methods described herein.

In some embodiments, the subject methods guiding a plurality of CRISPR-Cas effector complexes to a contiguous genomic region of interest further comprise imaging the genomic region of interest (e.g., imaging the genomic region of interest in a live cell). In some cases, the imaging may be accomplished using CRISPR-Cas complexes comprising fluorescently labeled guide RNAs (e.g., a fluorescently labeled guide RNA as described herein). In these and other embodiments of the methods the in vitro transcribed CRISPR-Cas guide RNAs may be labeled according to any of the subject labeling methods described herein.

In some embodiments, the subject methods guiding a plurality of CRISPR-Cas effector complexes to a contiguous genomic region of interest further comprise perturbing or regulating the genomic region of interest, In some cases, the perturbation comprises modulating expression of the genomic region of interest or modulating expression of another genomic region that is functionally linked to said genomic region of interest (e.g., in the case where the genomic region of interest comprises an enhancer). In some cases, the perturbation may simply be accomplished directly by contacting the genomic region of interest with the plurality of CRISPR-Cas complexes comprising nuclease-dead CRISPR-Cas effector proteins (e.g., thereby occluding endogenous factors from the genomic region of interest). In some cases, the perturbation may be accomplished, at least in part, by the activity of heterologous polypeptides fused to the CRISPR-Cas effector polypeptides (e.g., in the case where the heterologous polypeptide is a transcriptional activator/repressor or a chromatin modifying enzyme such as a histone methyltransferase).

In some embodiments, the subject methods guiding a plurality of CRISPR-Cas effector complexes to a contiguous genomic region of interest further comprise altering the genomic region of interest, e.g., by generating breaks in and/or editing the sequence of the genomic region of interest. For example, the altering may be accomplished using CRISPR-Cas effector complexes comprising nuclease active CRISPR-Cas effector proteins. In some cases, the altering may be accomplished, at least in part, by the activity of heterologous polypeptides fused to the CRISPR-Cas effector polypeptides (e.g., in the case where the heterologous polypeptide is a deaminase).

### CRISPR-Cas Effector Proteins

As discussed above, the present disclosure provides methods for guiding a plurality of CRISPR-Cas effector complexes to a contiguous genomic region of interest, wherein the each CRISPR-Cas effector complex in the plurality of complexes comprises: (a) a CRISPR-Cas effector protein; and (b) an in vitro transcribed guide RNA that hybridizes to a target sequence within the contiguous genomic region.

Examples of CRISPR-Cas effector proteins (also referred to as “CRISPR-Cas effector polypeptides” herein) suitable for use in the subject methods are CRISPR-Cas endonucleases (e.g., class 2 CRISPR-Cas effector polypeptide such as a type II, or type V, CRISPR-Cas effector polypeptide). Where a CRISPR-Cas effector polypeptide has endonuclease activity, the CRISPR-Cas effector polypeptide may also be referred to as a “CRISPR-Cas endonuclease.” A CRISPR-Cas effector polypeptide can also have reduced or undetectable endonuclease activity. A CRISPR-Cas effector polypeptide can also be a fusion CRISPR-Cas effector polypeptide comprising a heterologous fusion partner. In some cases, a suitable CRISPR-Cas effector polypeptide is a class 2 CRISPR-Cas effector polypeptide. In some cases, a suitable CRISPR-Cas effector polypeptide is a class 2 type II CRISPR-Cas effector polypeptide (e.g., a Cas9 protein). In some cases, a suitable CRISPR-Cas effector polypeptide is a class 2 type V CRISPR-Cas endonuclease (e.g., a Cpf1 protein, a C2c1 protein, or a C2c3 protein).

In class 2 CRISPR systems, the functions of the effector complex (e.g., the cleavage of target DNA) are carried out by a single endonuclease (e.g., see Zetsche et al., Cell. 2015 Oct. 22; 163 (3): 759-71; Makarova et al., Nat Rev Microbiol. 2015 November; 13 (11): 722-36; Shmakov et al., Mol Cell. 2015 Nov. 5; 60 (3): 385-97); and Shmakov et al. (2017) Nature Reviews Microbiology 15:169. As such, the term “class 2 CRISPR-Cas protein” is used herein to encompass the CRISPR-Cas effector polypeptide (e.g., the target nucleic acid cleaving protein) from class 2 CRISPR systems. Thus, the term “class 2 CRISPR-Cas effector polypeptide” as used herein encompasses type II CRISPR-Cas effector polypeptides (e.g., Cas9); type V-A CRISPR-Cas effector polypeptides (e.g., Cpf1 (also referred to a “Cas12a”)); type V-B CRISPR-Cas effector polypeptides (e.g., C2c1 (also referred to as “Cas12b”)); type V-C CRISPR-Cas effector polypeptides (e.g., C2c3 (also referred to as “Cas12c”)); type V-U1 CRISPR-Cas effector polypeptides (e.g., C2c4); type V-U2 CRISPR-Cas effector polypeptides (e.g., C2c8); type V-U5 CRISPR-Cas effector polypeptides (e.g., C2c5); type V-U4 CRISPR-Cas proteins (e.g., C2c9); type V-U3 CRISPR-Cas effector polypeptides (e.g., C2c10). To date, class 2 CRISPR-Cas effector polypeptides encompass type II, type V, and type VI CRISPR-Cas effector polypeptides, but the term is also meant to encompass any class 2 CRISPR-Cas effector polypeptide suitable for binding to a corresponding guide RNA and forming an RNP complex.

In some cases, a subject CRISPR-Cas effector polypeptide is a variant effector polypeptide. Variants of the effectors polypeptides (relative to wild type sequences) are also encompassed as long as a cognate guide RNA can form a complex with the effector and together they target a target sequence of a target DNA-determined by the guide sequence of the guide RNA (taking PAM into account). In some cases, the effector is a wild type protein that has DNA cleavage activity. In some cases, the effector protein is a mutant protein that has reduced activity (e.g., is a nickase or a catalytically inactive, i.e., dead version of the protein). In some cases, the effector is a type II nickase (e.g., RuvC domain or HNH domain catalytically inactive) or a Type II nuclease inactive protein (also referred to in the art as “dead” or “d” such as dCas, e.g., dCas9) (e.g., RuvC domain and HINH domain catalytically inactive).

In some cases, the CRISPR-Cas effector polypeptide (i.e., CRISPR-Cas effector protein) is a type II CRISPR-Cas effector polypeptide. In some cases, the type II CRISPR-Cas effector polypeptide is a Cas9 polypeptide, e.g., Staphylococcus aureus Cas9 (SaCas9), Streptococcus pyogenes Cas9 (SpCas9), etc. In some cases, the CRISPR-Cas effector polypeptide is a variant of a wild-type SpCas9 and comprises one or more of the following substitutions: A61R, L1111R, A1322R, D1135L, S1136W, G1218K, E1219Q, N1317R, R1333P, R1335A, and T1337R. In some cases, the CRISPR-Cas effector polypeptide is an SpG polypeptide or a SpRY polypeptide; see, e.g., Walton et al. (2020) Science 368:290, and WO 2019/051097. For example, a suitable CRISPR-Cas effector polypeptide is an SpCas9 polypeptide includes D1135V, R1135Q, and T1137R substitutions, relative to wild-type SpCas9. As another example, a suitable CRISPR-Cas effector polypeptide is an SpCas9 polypeptide includes D1135V, R1335Q, T1337R, and G1218R substitutions, relative to wild-type SpCas9. As another example, a suitable CRISPR-Cas effector polypeptide is an SpCas9 polypeptide includes D1135L, S1136W, G1218K, E1219Q, R1335A, and T1337R substitutions, relative to wild-type SpCas9. As another example, a suitable CRISPR-Cas effector polypeptide is an SpCas9 polypeptide includes L1111R, A1322R, D1135L, S1136W, G1218K, E1219Q, R1335A, and T1337R substitutions, relative to wild-type SpCas9. As another example, a suitable CRISPR-Cas effector polypeptide is an SpCas9 polypeptide includes A61R, L1111R, A1322R, D1135L, S1136W, G1218K, E1219Q, N1317R, R1333P, R1335A, and T1337R substitutions, relative to wild-type SpCas9.

In some cases, the CRISPR-Cas effector polypeptide is a CRISPRi polypeptide; see, e.g., Qi et al. (2013) Cell 152:1173; and Jensen et al. (2021) Genome Research doi: 10.1101/gr.275607.121. In some cases, the CRISPR-Cas effector polypeptide is a CRISPRa polypeptide; see, e.g., Jensen et al. (2021) Genome Research doi: 10.1101/gr.275607.121; and Breinig et al. (2019) Nature Methods 16:51. In some cases, the CRISPR-Cas effector polypeptide is a CRISPRoff polypeptide. See, e.g., Nuñez et al. (2021) Cell 184:2503. In some cases, the CRISPR-Cas effector polypeptide is a nickase. In some cases, the CRISPR-Cas effector polypeptide exhibits reduced catalytic activity compared to a wild-type CRISPR-Cas effector polypeptide.

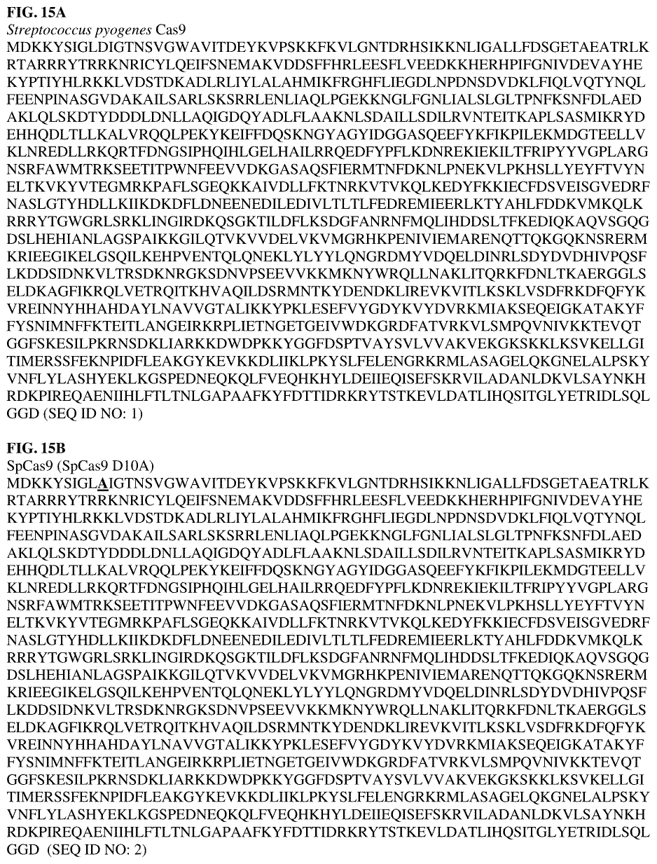

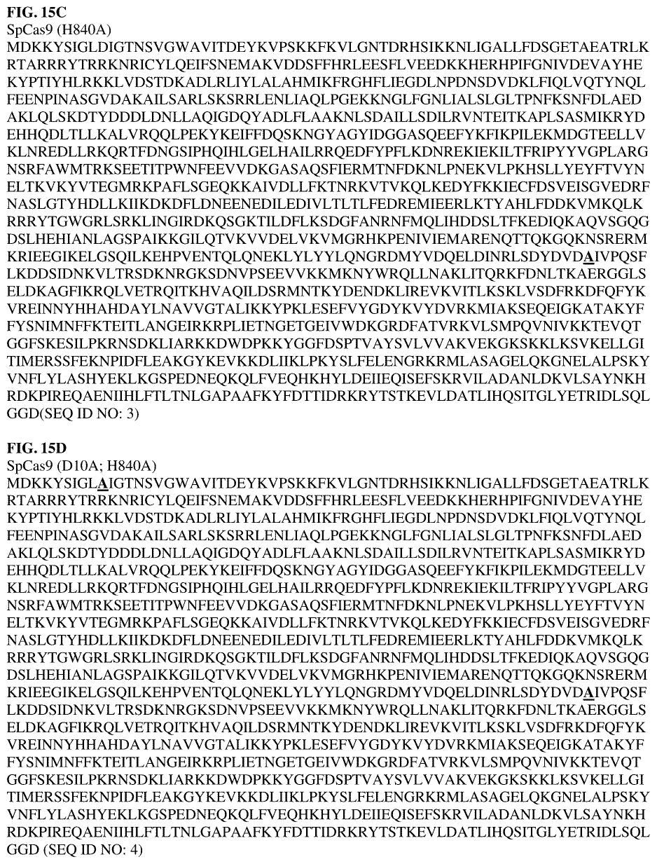

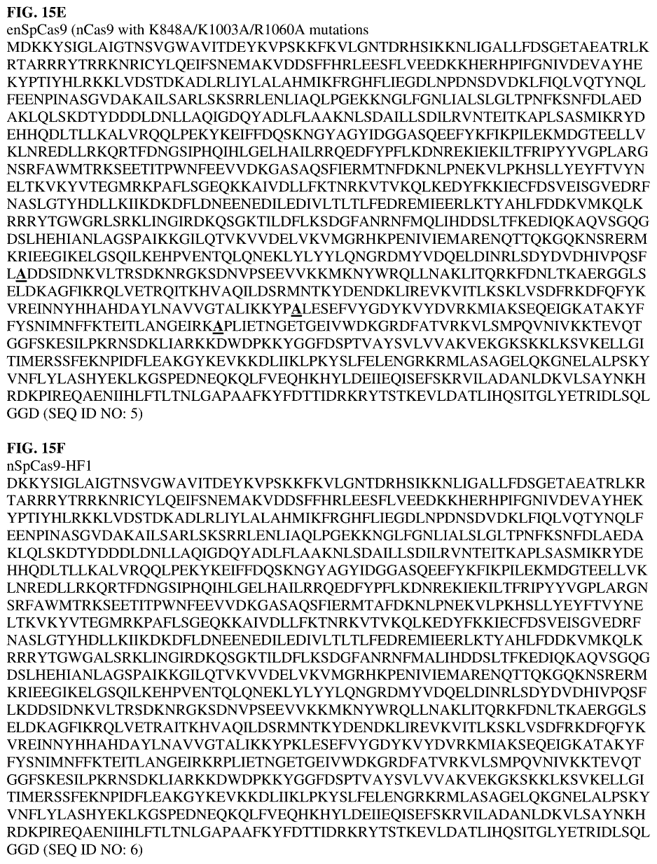

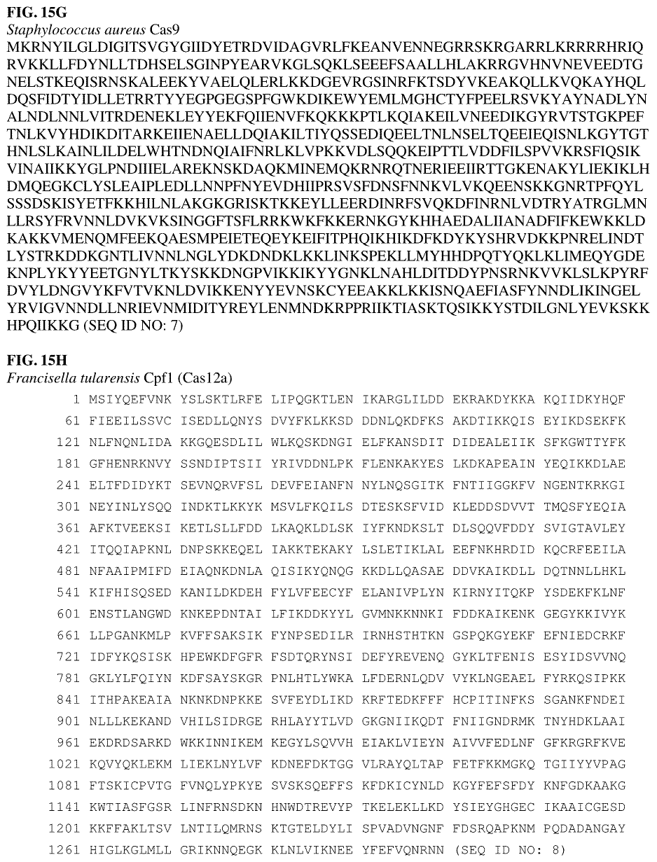

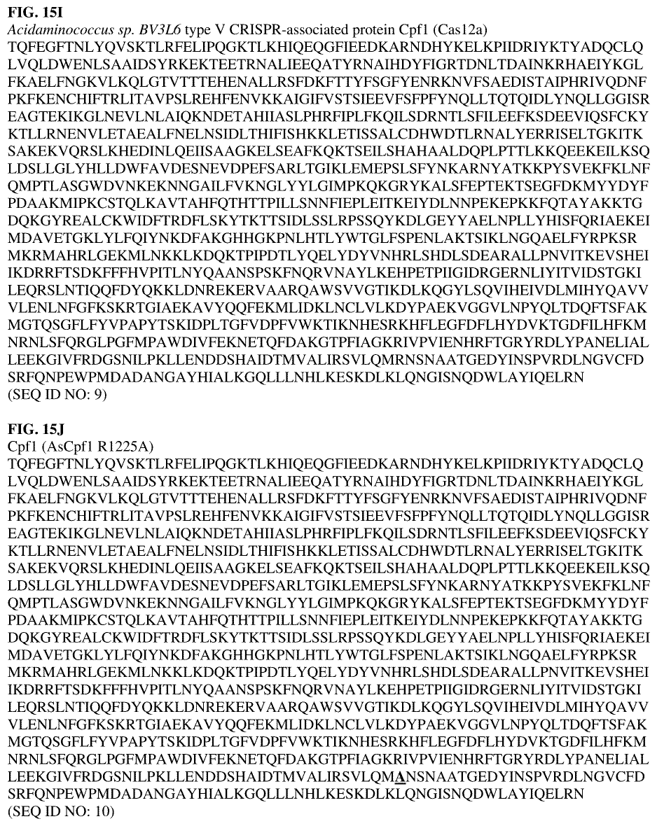

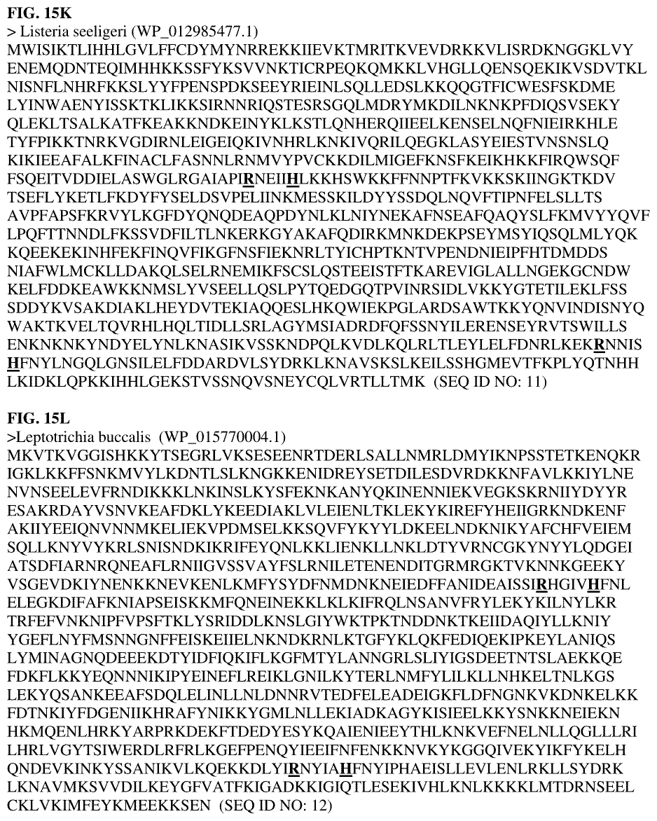

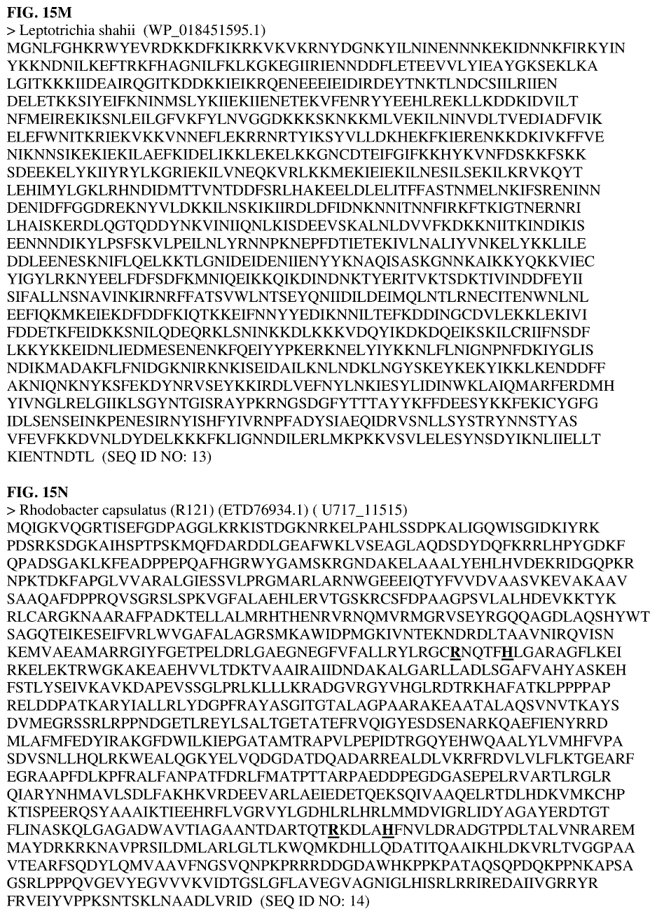

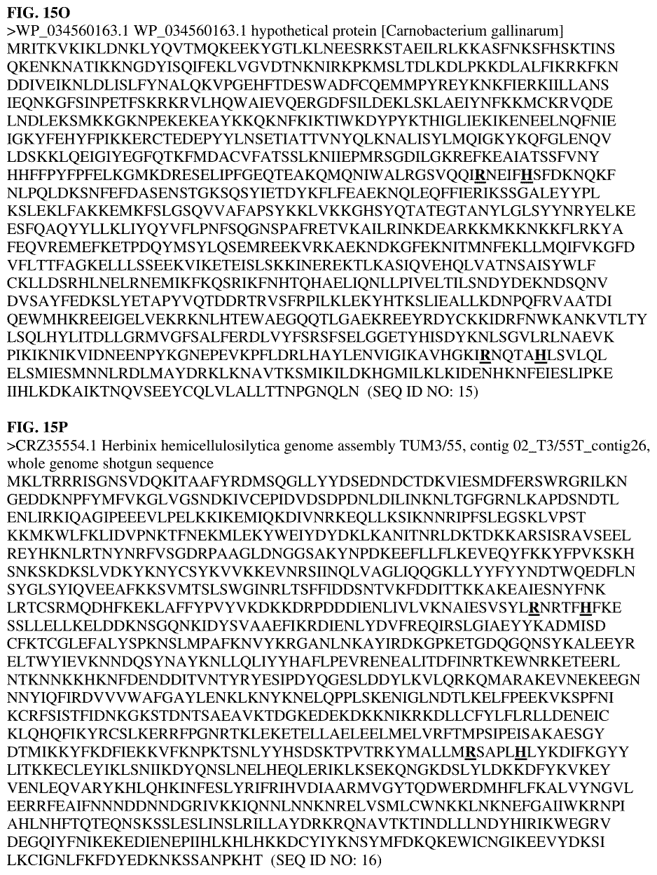

In some cases, a suitable CRISPR-Cas effector polypeptide comprises an amino acid sequence having at least 85%, at least 90%, at least 95%, at least 98%, at least 99%, or 100%, amino acid sequence identity to the amino acid sequence depicted in any one of FIG. 15A-15P.

In some cases, the CRISPR-Cas effector polypeptide is a Type II CRISPR-Cas effector polypeptide. In some cases, the CRISPR-Cas effector polypeptide is a Cas9 polypeptide. The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, a Cas9 polypeptide comprises an amino acid sequence having at least 50%, at least 60%, at least 70%, at least 80%, at least 90%, at least 95%, at least 98%, at least 99%, or more than 99%, amino acid sequence identity to the Streptococcus pyogenes Cas9 depicted in FIG. 15A.

In some cases, the Cas9 polypeptide is a Staphylococcus aureus Cas9 (saCas9) polypeptide. In some cases, the saCas9 polypeptide comprises an amino acid sequence having at least 85%, at least 90%, at least 95%, at least 98%, at least 99%, or 100%, amino acid sequence identity to the saCas9 amino acid sequence depicted in FIG. 15G.

In some cases, a suitable Cas9 polypeptide is a high-fidelity (HF) Cas9 polypeptide. Kleinstiver et al. (2016) Nature 529:490. For example, amino acids N497, R661, Q695, and Q926 of the amino acid sequence depicted in FIG. 15A are substituted, e.g., with alanine. For example, an HF Cas9 polypeptide can comprise an amino acid sequence having at least 90%, at least 95%, at least 98%, at least 99%, or 100%, amino acid sequence identity to the amino acid sequence depicted in FIG. 15A, where amino acids N497, R661, Q695, and Q926 are substituted, e.g., with alanine. In some cases, a suitable Cas9 polypeptide exhibits altered PAM specificity. See, e.g., Kleinstiver et al. (2015) Nature 523:481.

In some cases, a suitable CRISPR-Cas effector polypeptide is a type V CRISPR-Cas effector polypeptide. In some cases, a type V CRISPR-Cas effector polypeptide is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in FIG. 15H, FIG. 15I, or FIG. 15J.

In some cases, a suitable CRISPR-Cas effector polypeptide is a fusion protein comprising a CRISPR-Cas effector polypeptide that is fused to a heterologous polypeptide (also referred to as a “fusion partner”). In some cases, a CRISPR-Cas effector polypeptide is fused to an amino acid sequence (a fusion partner) that provides for subcellular localization, i.e., the fusion partner is a subcellular localization sequence (e.g., one or more nuclear localization signals (NLSs) for targeting to the nucleus, two or more NLSs, three or more NLSs, etc.).

In some cases, a CRISPR-Cas effector polypeptide is a fusion polypeptide comprising: i) a CRISPR-Cas effector polypeptide; and ii) one or more heterologous fusion partners (one or more heterologous fusion polypeptides). In some cases, a fusion CRISPR-Cas effector polypeptide comprises one or more localization signal peptides. In some cases, a fusion CRISPR-Cas effector polypeptide comprises one or more localization signal peptides. Suitable localization signals (“subcellular localization signals”) include, e.g., a nuclear localization signal (NLS) for targeting to the nucleus; a sequence to keep the fusion protein out of the nucleus, e.g., a nuclear export sequence (NES); a sequence to keep the fusion protein retained in the cytoplasm; a mitochondrial localization signal for targeting to the mitochondria; a chloroplast localization signal for targeting to a chloroplast; an endoplasmic reticulum (ER) retention signal; and ER export signal; and the like. In some cases, a fusion CRISPR-Cas effector polypeptide does not include a NLS so that the protein is not targeted to the nucleus (which can be advantageous, e.g., when the target nucleic acid is an RNA that is present in the cytosol). In some cases, a fusion CRISPR-Cas effector polypeptide comprises both an NES and one or more NLSs. A suitable NES comprises hydrophobic amino acid residues, e.g., LXXXLXXLXL, where L is a hydrophobic amino acid residue (e.g., Leu) and X is any other amino acid. Suitable NESs are known in the art; see, e.g., Xu et al. (2012) Mol. Biol. Cell 23:3677. Non-limiting examples of suitable NESs include: LPPLERLTL (SEQ ID NO: 19); LALKLAGLDL (SEQ ID NO: 20); LSQALASSFSV (SEQ ID NO: 21); NELALKLAGLDI (SEQ ID NO: 22). In some cases, the NES comprises the amino acid sequence LPPLERLTL (SEQ ID NO: 23).

In some cases, a fusion CRISPR-Cas effector polypeptide includes (is fused to) a nuclear localization signal (NLS) (e.g., in some cases 2 or more, 3 or more, 4 or more, or 5 or more NLSs). Thus, in some cases, a fusion polypeptide includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

Non-limiting examples of NLSs include an NLS sequence derived from: the NLS of the SV40 virus large T-antigen, having the amino acid sequence PKKKRKV (SEQ ID NO:24); the NLS from nucleoplasmin (e.g., the nucleoplasmin bipartite NLS with the sequence KRPAATKKAGQAKKKK (SEQ ID NO: 25)); the c-myc NLS having the amino acid sequence PAAKRVKLD (SEQ ID NO: 26) or RQRRNELKRSP (SEQ ID NO: 27); the hRNPA1 M9 NLS having the sequence NQSSNFGPMKGGNFGGRSSGPYGGGGQYFAKPRNQGGY (SEQ ID NO: 28); the sequence RMRIZFKNKGKDTAELRRRRVEVSVELRKAKKDEQILKRRNV (SEQ ID NO: 29) of the IBB domain from importin-alpha; the sequences VSRKRPRP (SEQ ID NO: 30) and PPKKARED (SEQ ID NO: 31) of the myoma T protein; the sequence PQPKKKPL (SEQ ID NO: 32) of human p53; the sequence SALIKKKKKMAP (SEQ ID NO: 33) of mouse c-abl IV; the sequences DRLRR (SEQ ID NO: 34) and PKQKKRK (SEQ ID NO: 35) of the influenza virus NS1; the sequence RKLKKKIKKL (SEQ ID NO: 36) of the Hepatitis virus delta antigen; the sequence REKKKFLKRR (SEQ ID NO: 37) of the mouse Mx1 protein; the sequence KRKGDEVDGVDEVAKKKSKK (SEQ ID NO: 38) of the human poly (ADP-ribose) polymerase; and the sequence RKCLQAGMNLEARKTKK (SEQ ID NO: 39) of the steroid hormone receptors (human) glucocorticoid. In some cases, an NLS comprises the amino acid sequence MDSLLMNRRKFLYQFKNVRWAKGRRETYLC (SEQ ID NO: 40). In general, NLS (or multiple NLSs) are of sufficient strength to drive accumulation of the fusion polypeptide in a detectable amount in the nucleus of a eukaryotic cell.

### Fusion Polypeptides

The present disclosure provides methods for guiding a plurality of CRISPR-Cas effector complexes to a contiguous genomic region of interest, wherein the each CRISPR-Cas effector complex in the plurality of complexes comprises: (a) a CRISPR-Cas effector protein; and (b) an in vitro transcribed guide RNA that hybridizes to a target sequence within the contiguous genomic region. In some embodiments, the CRISPR-Cas effector protein comprises a heterologous polypeptide (also referred to as a “fusion partner” herein). The CRISPR-Cas effector protein comprising a heterologous polypeptide is also sometimes referred to as a “CRISPR-Cas fusion polypeptide”.

In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases, the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like). In some cases, the fusion partner is a reverse transcriptase. In some cases, the fusion partner is a base editor. In some cases, the fusion partner is a deaminase.

In some cases, a CRISPR-Cas fusion polypeptide includes a heterologous polypeptide that has enzymatic activity that modifies a target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, or glycosylase activity).

In some cases, a CRISPR-Cas fusion polypeptide includes a heterologous polypeptide that has enzymatic activity that modifies a polypeptide (e.g., a histone) associated with a target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Examples of proteins (or fragments thereof) that can be used in increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like.

Examples of proteins (or fragments thereof) that can be used in decrease transcription include but are not limited to: transcriptional repressors such as the Krüppel associated box (KRAB or SKD); KOXI repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARIDIA/RBP2, JARID1B/PLU-1, JARID1C/SMCX, JARIDID/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some cases, the fusion partner has enzymatic activity that modifies the target nucleic acid Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like); demethylase activity such as that provided by a demethylase (e.g., Ten-Eleven Translocation (TET) dioxygenase 1 (TET1CD), TET1, DME, DML1, DML2, ROS1, and the like), DNA repair activity, DNA damage activity, deamination activity such as that provided by a deaminase (e.g., a cytosine deaminase enzyme such as rat APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In some cases, the fusion partner has enzymatic activity that modifies a protein associated with the target nucleic acid (e.g., a histone, a DNA binding protein, and the like). Examples of enzymatic activity (that modifies a protein associated with a target nucleic acid) that can be provided by the fusion partner include but are not limited to: methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, and the like, SET1A, SET1B, MLL1 to 5, ASH1, SYMD2, NSD1, DOTIL, Pr-SET7/8, SUV4-20H1, EZH2, RIZ1), demethylase activity such as that provided by a histone demethylase (e.g., Lysine Demethylase 1A (KDMIA also known as LSD1), JHDM2a/b, JMJD2A/JHDM3A, JMJD2B, JMJD2C/GASC1, JMJD2D, JARIDIA/RBP2, JARID1B/PLU-1, JARID1C/SMCX, JARID1D/SMCY, UTX, JMJD3, and the like), acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK, and the like), deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

Additional examples of a suitable fusion partners are dihydrofolate reductase (DHFR) destabilization domain (e.g., to generate a chemically controllable fusion polypeptide), and a chloroplast transit peptide.

In some case, a CRISPR-Cas fusion polypeptide comprises: a) a CRISPR-Cas effector polypeptide; and b) a chloroplast transit peptide. Thus, for example, a ribonucleoprotein (RNP) complex, comprising a CRISPR-Cas effector polypeptide of the present disclosure and a guide RNA, can be targeted to the chloroplast. In some cases, this targeting may be achieved by the presence of an N-terminal extension, called a chloroplast transit peptide (CTP) or plastid transit peptide. Chromosomal transgenes from bacterial sources must have a sequence encoding a CTP sequence fused to a sequence encoding an expressed polypeptide if the expressed polypeptide is to be compartmentalized in the plant plastid (e.g. chloroplast). Accordingly, localization of an exogenous polypeptide to a chloroplast is often 1 accomplished by means of operably linking a polynucleotide sequence encoding a CTP sequence to the 5′ region of a polynucleotide encoding the exogenous polypeptide. The CTP is removed in a processing step during translocation into the plastid. Processing efficiency may, however, be affected by the amino acid sequence of the CTP and nearby sequences at the amino terminus of the peptide. Other options for targeting to the chloroplast which have been described are the maize cab-m7 signal sequence (U.S. Pat. No. 7,022,896, WO 97/41228) a pea glutathione reductase signal sequence (WO 97/41228) and the CTP described in US2009029861.

In some cases, a CRISPR-Cas fusion polypeptide comprises: a) a CRISPR-Cas effector polypeptide of the present disclosure; and b) an endosomal escape peptide. In some cases, an endosomal escape polypeptide comprises the amino acid sequence GLFXALLXLLXSLWXLLLXA (SEQ ID NO: 41), wherein each X is independently selected from lysine, histidine, and arginine. In some cases, an endosomal escape polypeptide comprises the amino acid sequence GLFHALLHLLHSLWHLLLHA (SEQ ID NO:42).

Additional suitable heterologous polypeptides include, but are not limited to, a polypeptide that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). Non-limiting examples of heterologous polypeptides to accomplish increased or decreased transcription include transcription activator and transcription repressor domains. In some such cases, a CRISPR-Cas fusion polypeptide is targeted by the guide nucleic acid (guide RNA) to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target nucleic acid or modifies a polypeptide associated with the target nucleic acid). In some cases, the changes are transient (e.g., transcription repression or activation). In some cases, the changes are inheritable (e.g., when epigenetic modifications are made to the target nucleic acid or to proteins associated with the target nucleic acid, e.g., nucleosomal histones).

Further suitable fusion partners include, but are not limited to, proteins (or fragments thereof) that are boundary elements (e.g., CTCF), proteins and fragments thereof that provide periphery recruitment (e.g., Lamin A, Lamin B, etc.), protein docking elements (e.g., FKBP/FRB, Pill/Abyl, etc.).

In some cases, a CRISPR-Cas fusion polypeptide comprises a nucleic acid binding effector polypeptide. Suitable nucleic acid binding effector polypeptides can be nucleases including, but not limited to, a homing nuclease polypeptide; a FokI polypeptide; a MegaTAL polypeptide; a meganuclease polypeptide; an ARCUS nuclease; and the like. The meganuclease can be engineered from an LADLIDADG homing endonuclease (LHE). A megaTAL polypeptide can comprise a TALE DNA binding domain and an engineered meganuclease. See, e.g., WO 2004/067736 (homing endonuclease); Urnov et al. (2005) Nature 435:646 (ZFN); Mussolino et al. (2011) Nucle. Acids Res. 39:9283 (TALE nuclease); Boissel et al. (2013) Nucl. Acids Res. 42:2591 (MegaTAL).

In some cases, a CRISPR-Cas fusion polypeptide comprises a recombinase. Suitable recombinases include, e.g., a Cre recombinase; a Hin recombinase; a Tre recombinase; a FLP recombinase; and the like.

In some cases, a CRISPR-Cas fusion polypeptide includes a “Protein Transduction Domain” or PTD (also known as a CPP-cell penetrating peptide), which refers to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some cases, a PTD is covalently linked to the amino terminus of a CRISPR-Cas effector polypeptide. In some cases, a PTD is covalently linked to the carboxyl terminus of a CRISPR-Cas effector polypeptide. In some cases, the PTD is inserted internally in a CRISPR-Cas effector polypeptide (i.e., is not at the N- or C-terminus of the CRISPR-Cas effector polypeptide) at a suitable insertion site. In some cases, a CRISPR-Cas fusion polypeptide includes: a) a CRISPR-Cas fusion polypeptide; and b) one or more PTDs (e.g., two or more, three or more, four or more PTDs). In some cases, a PTD includes a nuclear localization signal (NLS) (e.g., in some cases 2 or more, 3 or more, 4 or more, or 5 or more NLSs). Thus, in some cases, a CRISPR-Cas fusion polypeptide includes one or more NLSs (e.g., 2 or more, 3 or more, 4 or more, or 5 or more NLSs). In some cases, a PTD is covalently linked to a nucleic acid (e.g., a CRISPR-Cas guide nucleic acid, a polynucleotide encoding a CRISPR-Cas guide nucleic acid, a polynucleotide encoding a fusion polypeptide, a donor polynucleotide, etc.). Examples of PTDs include but are not limited to a minimal undecapeptide protein transduction domain (corresponding to residues 47-57 of HIV-1 TAT comprising YGRKKRRQRRR; SEQ ID NO: 55); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9 (6): 489-96); a Drosophila Antennapedia protein transduction domain (Noguchi et al. (2003) Diabetes 52 (7): 1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO: 56); Transportan GWTLNSAGYLLGKINLKALAALAKKIL (SEQ ID NO: 57); KALAWEAKLAKALAKALAKHLAKALAKALKCEA (SEQ ID NO: 58); and RQIKIWFQNRRMKWKK (SEQ ID NO: 59). Exemplary PTDs include but are not limited to, YGRKKRRQRRR (SEQ ID NO: 55), RKKRRQRRR (SEQ ID NO: 60); an arginine homopolymer of from 3 arginine residues to 50 arginine residues; Exemplary PTD domain amino acid sequences include, but are not limited to, any of the following: YGRKKRRQRRR (SEQ ID NO: 55); RKKRRQRR (SEQ ID NO: 61); YARAAARQARA (SEQ ID NO: 62); THRLPRRRRRR (SEQ ID NO: 63); and GGRRARRRRRR (SEQ ID NO: 64). In some cases, the PTD is an activatable CPP (ACPP) (Aguilera et al. (2009) Integr Biol (Camb) June; 1 (5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

In some cases, a CRISPR-Cas fusion polypeptide comprises a base editor. Suitable base editors include deaminase such as, e.g., an adenosine deaminase; a cytidine deaminase (e.g., an activation-induced cytidine deaminase (AID)); APOBEC3G; and the like); and the like. A suitable adenosine deaminase is any enzyme that is capable of deaminating adenosine in DNA. In some cases, the deaminase is a TadA deaminase.

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 43)
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD.

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 44)
MRRAFITGVFFLSEVEFSHEYWMRHALTLAKRAWDEREVPVGAVLVHNNR
VIGEGWNRPIGRHDPTAHAEIMALRQGGLVMQNYRLIDATLYVTLEPCVM
CAGAMIHSRIGRVVFGARDAKTGAAGSLMDVLHHPGMNHRVEITEGILAD
ECAALLSDFFRMRRQEIKAQKKAQSSTD.

In some cases, a suitable adenosine deaminase comprises an amino acid sequence having at least 80%, at least 85%, at least 90%, at least 95%, at least 98%, at least 99%, or 100%, amino acid sequence identity to the following Staphylococcus aureus TadA amino acid sequence:

(SEQ ID NO: 45)
MGSHMTNDIYFMTLAIEEAKKAAQLGEVPIGAIITKDDEVIARAHNLRET
LQQPTAHAEHIAIERAAKVLGSWRLEGCTLYVTLEPCVMCAGTIVMSRIP
RVVYGADDPKGGCSGSLMNLLQQSNFNHRAIVDKGVLKEACSTLLTTFFK
NLRANKKSTN.

In some cases, a suitable adenosine deaminase comprises an amino acid sequence having at least 80%, at least 85%, at least 90%, at least 95%, at least 98%, at least 99%, or 100%, amino acid sequence identity to the following Bacillus subtilis TadA amino acid sequence:

(SEQ ID NO: 46)
MTQDELYMKEAIKEAKKAEEKGEVPIGAVLVINGEIIARAHNLRETEQRS
IAHAEMLVIDEACKALGTWRLEGATLYVTLEPCPMCAGAVVLSRVEKVVF
GAFDPKGGCSGTLMNLLQEERFNHQAEVVSGVLEEECGGMLSAFFRELRK
KKKAARKNLSE.

In some cases, a suitable adenosine deaminase comprises an amino acid sequence having at least 80%, at least 85%, at least 90%, at least 95%, at least 98%, at least 99%, or 100%, amino acid sequence identity to the following Salmonella typhimurium TadA:

(SEQ ID NO: 47)
MPPAFITGVTSLSDVELDHEYWMRHALTLAKRAWDEREVPVGAVLVHNHR
VIGEGWNRPIGRHDPTAHAEIMALRQGGLVLQNYRLLDTTLYVTLEPCVM
CAGAMVHSRIGRVVFGARDAKTGAAGSLIDVLHHPGMNHRVEIIEGVLRD
ECATLLSDFFRMRRQEIKALKKADRAEGAGPAV.

In some cases, a suitable adenosine deaminase comprises an amino acid sequence having at least 80%, at least 85%, at least 90%, at least 95%, at least 98%, at least 99%, or 100%, amino acid sequence identity to the following Shewanella putrefaciens TadA amino acid sequence:

(SEQ ID NO: 48)
MDEYWMQVAMQMAEKAEAAGEVPVGAVLVKDGQQIATGYNLSISQHDPTA
HAEILCLRSAGKKLENYRLLDATLYITLEPCAMCAGAMVHSRIARVVYGA
RDEKTGAAGTVVNLLQHPAFNHQVEVTSGVLAEACSAQLSRFFKRRRDEK
KALKLAQRAQQGIE.

In some cases, a suitable adenosine deaminase comprises an amino acid sequence having at least 80%, at least 85%, at least 90%, at least 95%, at least 98%, at least 99%, or 100%, amino acid sequence identity to the following Haemophilus influenzae F3031 TadA amino acid sequence:

(SEQ ID NO: 49)
MDAAKVRSEFDEKMMRYALELADKAEALGEIPVGAVLVDDARNIIGEGWN
LSIVQSDPTAHAEIIALRNGAKNIQNYRLLNSTLYVTLEPCTMCAGAILH
SRIKRLVFGASDYKTGAIGSRFHFFDDYKMNHTLEITSGVLAEECSQKLS
TFFQKRREEKKIEKALLKSLSDK.

In some cases, a suitable adenosine deaminase comprises an amino acid sequence having at least 80%, at least 85%, at least 90%, at least 95%, at least 98%, at least 99%, or 100%, amino acid sequence identity to the following Caulobacter crescentus TadA amino acid sequence:

(SEQ ID NO: 50)
MRTDESEDQDHRMMRLALDAARAAAEAGETPVGAVILDPSTGEVIATAGN
GPIAAHDPTAHAEIAAMRAAAAKLGNYRLTDLTLVVTLEPCAMCAGAISH
ARIGRVVFGADDPKGGAVVHGPKFFAQPTCHWRPEVTGGVLADESADLLR
GFFRARRKAKI.

In some cases, a suitable adenosine deaminase comprises an amino acid sequence having at least 80%, at least 85%, at least 90%, at least 95%, at least 98%, at least 99%, or 100%, amino acid sequence identity to the following Geobacter sulfurreducens TadA amino acid sequence:

(SEQ ID NO: 51)
MSSLKKTPIRDDAYWMGKAIREAAKAAARDEVPIGAVIVRDGAVIGRGHN
LREGSNDPSAHAEMIAIRQAARRSANWRLTGATLYVTLEPCLMCMGAIIL
ARLERVVFGCYDPKGGAAGSLYDLSADPRLNHQVRLSPGVCQEECGTMLS
DFFRDLRRRKKAKATPALFIDERKVPPEP.

Cytidine deaminases suitable for inclusion in a CRISPR-Cas effector polypeptide fusion polypeptide of the present disclosure include any enzyme that is capable of deaminating cytidine in DNA.

In some cases, the cytidine deaminase is a deaminase from the apolipoprotein B mRNA-editing complex (APOBEC) family of deaminases. In some cases, the APOBEC family deaminase is selected from the group consisting of APOBECI deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase. In some cases, the cytidine deaminase is an activation induced deaminase (AID).

In some cases, a suitable cytidine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 52)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKENHERTFK AWEGLHENSV RLSRQLRRIL LPLYEVDDLR DAFRTLGL (SEQ ID NO:53).

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKDYFYCWNT FVENHERTFK AWEGLHENSV RLSRQLRRIL LPLYEVDDLR DAFRTLGL (SEQ ID NO: 54).

In some cases, a CRISPR-Cas fusion polypeptide comprises a reverse transcriptase polypeptide. Reverse transcriptases are known in the art; see, e.g., Coté and Roth (2008) Virus Res. 134:186. Suitable reverse transcriptases include, e.g., a murine leukemia virus reverse transcriptase; a Rous sarcoma virus reverse transcriptase; a human immunodeficiency virus type I reverse transcriptase; a Moloney murine leukemia virus reverse transcriptase; a transcription xenopolymerase (RTX); avian myeloblastosis virus reverse transcriptase (AMV-RT); a Eubacterium rectale maturase reverse transcriptase (Marathon®; and the like. The reverse transcriptase fusion partner can include one or more mutations. For example, in some cases, the reverse transcriptase is a M-MLV reverse transcriptase polypeptide that comprises one or more mutations selected from the group consisting of D200N, T306K, W313F, T330P and L603W. In some cases, the reverse transcriptase is a pentamutant of M-MLV RT (e.g., comprising the following substitutions: D200N/L603W/T330P/T306K/W313F).

In some cases, a CRISPR-Cas fusion polypeptide comprises an antibody that may be used, for example, to isolate the genomic region of interest. Suitable antibodies include any desired antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, evibodies, minobodies, diabodies, and fusion proteins comprising an antigen-binding (also referred to herein as antigen binding) portion of an antibody and a non-antibody protein.

### Linkers (e.g., for Fusion Partners)

In some cases, a CRISPR-Cas polypeptide can be fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G)ₙ where n is an integer of at least one; glycine-serine polymers (including, for example, (GS)ₙ, (GSGGS)ₙ (SEQ ID NO: 65), (GGSGGS)ₙ (SEQ ID NO: 66), (GGGGS)n (SEQ ID NO:67), and (GGGS)ₙ (SEQ ID NO: 68), where n is an integer of at least one; e.g., where n is an integer from 1 to 10); glycine-alanine polymers; and alanine-serine polymers. Exemplary linkers can comprise amino acid sequences including, but not limited to, GGSG (SEQ ID NO: 69), GGSGG (SEQ ID NO: 70), GSGSG (SEQ ID NO: 71), GSGGG (SEQ ID NO: 72), GGGSG (SEQ ID NO: 73), GSSSG (SEQ ID NO: 74), GGGGS (SEQ ID NO:75), and the like. The ordinarily skilled artisan will recognize that design of a peptide conjugated to any desired element can include linkers that are all or partially flexible, such that the linker can include a flexible linker as well as one or more portions that confer less flexible structure.

### Target Cells

As discussed above, the present disclosure provides methods for guiding a plurality of CRISPR-Cas effector complexes to a contiguous genomic region of interest, the method comprising contacting a contiguous genomic region in a target cell with a plurality of CRISPR-Cas effector complexes.

Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be an in vivo cell (e.g., a cell in an individual). A cell can be an isolated cell. A cell can be a cell inside of an organism. A cell can be an organism. A cell can be a cell in a cell culture (e.g., in vitro cell culture). A cell can be one of a collection of cells. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a mammalian cell or derived from a mammalian cell. A cell can be a rodent cell or derived from a rodent cell. A cell can be a human cell or derived from a human cell. A cell can be a microbe cell or derived from a microbe cell. A cell can be a fungi cell or derived from a fungi cell. A cell can be an insect cell. A cell can be an arthropod cell. A cell can be a protozoan cell. A cell can be a helminth cell.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogeneic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

A cell is in some cases a plant cell. A plant cell can be a cell of a monocotyledon. A cell can be a cell of a dicotyledon.

In some cases, the cell is a plant cell. For example, the cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf-green), lettuce (oak leaf-red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

### Delivery of CRISPR-Cas Effector Complexes

As discussed above, the present disclosure provides methods for guiding a plurality of CRISPR-Cas effector complexes to a contiguous genomic region of interest, the method comprising contacting a contiguous genomic region in a target cell with a plurality of CRISPR-Cas effector complexes. The subject plurality of CRISPR-Cas effector complexes may be contacted with the target cell and delivered into the target cell via any convenient method; such methods are known to those of ordinary skill in the art. In some cases, the CRISPR-Cas complexes may be delivered to a target cell via transfection, nucleofection, conjugation, protoplast fusion, lipofection, nucleofection, electroporation, calcium phosphate precipitation, polyethyleneimine (PEI)-mediated transfection, DEAE-dextran mediated transfection, liposome-mediated transfection, particle gun technology, calcium phosphate precipitation, agrobacterium-mediated transformation, direct micro injection, nanoparticle-mediated nucleic acid delivery (see, e.g., Panyam et., al Adv Drug Deliv Rev. 2012 Sep. 13. pii: S0169-409X (12) 00283-9. doi: 10.1016/j.addr.2012.09.023), and the like.

### EXEMPLARY NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

1. A method for generating a list of chromatin accessible target sequences for CRISPR-Cas guide RNAs that target a contiguous genomic region, the method comprising using one or more computer processors to:
(1) generate a master list of all possible target sequences in a genomic region of interest for a PAM sequence of interest, wherein each target sequence is 13-20 nucleotides in length; and
(2) generate a list of chromatin accessible target sequences from said master list, via:
   (i) removing any target sequence from the master list that would result in any of the following sequences being contained in a CRISPR-Cas guide RNA that targets said target sequence: UUUU; (G) 10, CUCGAG, GCUNAGC, or CCANNNNNNUGG;
   (ii) removing any target sequence from the master list that has more than 100 off-target sites, wherein an off-target site is a sequence outside of the genomic region of interest that is adjacent the PAM sequence of interest and:
      (a) has 0 or 1 mismatch to the target sequence for target sequences that are less than 16 nucleotides in length; or
      (b) has 0, 1, or 2 mismatches to the target sequence for target sequences that are more than 15 nucleotides in length;
   (iii) removing any target sequence from the master list that has an off-target site in an off-target region, wherein an off-target region is a genomic region outside the genomic region of interest that:
      (a) is less than 2000 nucleotides in length; and
      (b) contains two or more off-target sites as defined in (2) (ii);
   (iv) after performing (i), (ii), and (iii): removing, in 5′ to 3′ order along the genomic region of interest, any remaining target sequence from the master list that is 3′ of, and within 10 nucleotides of, another remaining target sequence of the master list; and
   (v) selecting chromatin accessible target sequences from the remaining target sequences of the master list by:
      (a) providing one or more histone modification and/or ATAC-seq datasets corresponding to the genomic region of interest, wherein the one or more histone modification datasets comprise H3K27 acetylation enrichment peak data; and
      (b) selecting target sequences in the list within 5000 nucleotides in distance from an H3K27 acetylation peak and/or an ATAC-seq peak for inclusion in the list of target sequences,
thereby generating a list of chromatin accessible target sequences for CRISPR-Cas guide RNAs that target a genomic region of interest.

2. The method of any of the preceding claims, wherein the genomic region of interest is a non-repetitive genomic region.

3 The method of 1 or 2, wherein the genomic region of interest is at least 500 contiguous nucleotides in length.

4. The method of 3, wherein the genomic region of interest is 500 to 2,000,000 contiguous nucleotides in length.

5. The method of any one of 3-4, wherein the method generates a list of at least 3 chromatin accessible target sequences.

6. The method of any one of 3-5, wherein the method generates a list of 3 to 20,000 chromatin accessible target sequences.

7. The method of any one of 3-6, wherein the method generates a list of one chromatin accessible target sequence for every 100 bp-150 bp in the genomic region of interest.

8. The method of any one of 1-7, wherein the method further comprises producing a plurality of CRISPR-Cas guide RNAs that target sequences from the list of chromatin accessible target sequences.

9. The method of 8, wherein the producing comprises in vitro transcribing the plurality of CRISPR-Cas guide RNAs from a plurality of template nucleic acids.

10. The method of 9, wherein the template nucleic acids are linear single-stranded oligonucleotides.

11. A plurality of in vitro transcribed CRISPR-Cas guide RNAs, wherein each guide RNA comprises:
(a) a guide sequence that hybridizes to a target sequence; and
(b) a scaffold sequence that can bind to a CRISPR-Cas effector protein, wherein the plurality comprises 3 or more of said CRISPR-Cas guide RNAs that hybridize to different target sequences within a genomic region of interest at least 500 contiguous nucleotides in length.

12. The plurality of in vitro transcribed CRISPR-Cas guide RNAs of 11, wherein the plurality comprises 3 to 20,000 of said CRISPR-Cas guide RNAs that hybridize to different target sequences within the genomic region of interest.

13. The plurality of in vitro transcribed CRISPR-Cas guide RNAs of 12, wherein the plurality comprises 20 to 20,000 of said CRISPR-Cas guide RNAs that hybridize to different target sequences within the genomic region of interest.

14. The plurality of in vitro transcribed CRISPR-Cas guide RNAs of any one of 11-13, wherein the genomic region of interest is 500 to 2,000,000 contiguous nucleotides in length. 15. The plurality of in vitro transcribed CRISPR-Cas guide RNAs of any one of 11-14, wherein the genomic region of interest is a non-repetitive genomic region.

16. The plurality of in vitro transcribed CRISPR-Cas guide RNAs of any one of 11-15, wherein said guide RNAs target sequences selected from a list of chromatin accessible target sequences generated according to the method of any one of 1-7.

17. The plurality of in vitro transcribed CRISPR-Cas guide RNAs of any one of 11-16, wherein the guide RNAs are single-molecule guide RNAs such that (a) and (b) are present on the same nucleic acid molecule.

18. The plurality of in vitro transcribed CRISPR-Cas guide RNAs of any one of 11-16, wherein the guide RNAs are not single-molecule guide RNAs such that (a) and (b) are present on different nucleic acid molecules.

19. The plurality of in vitro transcribed CRISPR-Cas guide RNAs of 17, wherein the guide RNAs comprise a 3′ poly(A) tail, wherein at least one adenosine of the poly(A) tail comprises a heterologous moiety.

20. The plurality of in vitro transcribed CRISPR-Cas guide RNAs of 17 or 19, wherein the guide RNAs comprise a heterologous moiety at their 5′ end.

21. The plurality of in vitro transcribed CRISPR-Cas guide RNAs of 18 or 20, wherein the 5′ end of the nucleic molecule that comprises (a), and/or the 5′ end of the nucleic molecule that comprises (b), comprises a heterologous moiety.

22. The plurality of in vitro transcribed CRISPR-Cas guide RNAs of 18 or 20, wherein the nucleic molecule that comprises (a), and/or the nucleic molecule that comprises (b), comprises a 3′ poly(A) tail, wherein at least one adenosine of the poly(A) tail comprises a heterologous moiety.

23. The plurality of in vitro transcribed CRISPR-Cas guide RNAs of any one of 19-22, wherein the heterologous moiety comprises a fluorescent dye.

24. The plurality of in vitro transcribed CRISPR-Cas guide RNAs of 23, wherein the fluorescent dye is selected from: Cy3, Cy5, A488, A565, A647, and any derivatives thereof.

25. The plurality of in vitro transcribed CRISPR-Cas guide RNAs of any one of 11-24, wherein the CRISPR-Cas effector protein is a Class 2 CRISPR-Cas effector protein.

26. The plurality of in vitro transcribed CRISPR-Cas guide RNAs of 25, wherein the Class 2 CRISPR-Cas effector protein is a Type II or Type V CRISPR-Cas effector protein.

27. A CRISPR-Cas guide RNA comprising:
(a) a guide sequence that hybridizes to a target sequence;
(b) a scaffold sequence that can bind to a CRISPR-Cas effector protein; and
(c) a 3′ poly(A) tail, wherein at least one adenosine of the poly(A) tail comprises a heterologous moiety.

28. The CRISPR-Cas guide RNA of 27, wherein the guide RNA is a single-molecule guide RNA such that (a) and (b) are present on the same nucleic acid molecule.

29. The CRISPR-Cas guide RNA of 27, wherein the guide RNA is not a single-molecule guide RNA such that (a) and (b) are present on different nucleic acid molecules.

30. The CRISPR-Cas guide RNA of 29, wherein the nucleic acid that comprises (b) comprises the 3′ poly(A) tail.

31. The CRISPR-Cas guide RNA of any one of 27-30, where said at least one adenosine is an azido-modified adenosine.

32. The CRISPR-Cas guide RNA of any one of 27-31, wherein the heterologous moiety comprises a fluorescent dye.

33. The CRISPR-Cas guide RNA of 32, wherein the fluorescent dye is selected from: Cy3, Cy5, A488, A565, A647, and any derivatives thereof.

34. The CRISPR-Cas guide RNA of any one of 27-33, wherein the CRISPR-Cas effector protein is a Class 2 CRISPR-Cas effector protein.

35. The CRISPR-Cas guide RNA of 34, wherein the Class 2 CRISPR-Cas effector protein is a Type II or Type V CRISPR-Cas effector protein.

36. The CRISPR-Cas guide RNA of any one of 27-35, wherein said guide RNAs target a target sequence selected from a list of chromatin accessible target sequences generated according to the method of any one of 1-7.

37. A method of labeling a CRISPR-Cas guide RNA, the method comprising: contacting a CRISPR-Cas guide RNA with a poly(A) polymerase and adenosine triphosphate (ATP) molecules, wherein at least one ATP molecule comprises a heterologous moiety, thereby generating a CRISPR-Cas guide RNA comprising a poly(A) tail in which at least one adenosine of the poly(A) tail comprises the heterologous moiety.

38. The method of 37, wherein the heterologous moiety is an azide moiety.

39. The method 37 or 38, wherein the method further comprises, conjugating a fluorescent dye to the heterologous moiety.

40. The method of 39, wherein the fluorescent dye comprises an azadibenzylcyclooctyne (DBCO) moiety.

41. The method of any one of 39-40, wherein the conjugation is performed via Click chemistry.

42. The method of any one of 39-41, wherein the fluorescent dye is selected from: Cy3, Cy5, A488, A565, A647, and any derivatives thereof.

43. A plurality of guide RNA template nucleic acids, each template nucleic acid comprising:
(a) a first primer binding domain common to a subset of the plurality of template nucleic acids;
(b) an RNA-polymerase promoter; and
(c) a sequence encoding a guide RNA, wherein the guide RNA comprises:
   (i) a guide sequence that hybridizes to a target sequence; and
   (ii) a sequence common to the plurality of template nucleic acids,
wherein the guide sequences encoded in the template nucleic acids hybridize to target sequences within a genomic region of interest at least 500 contiguous nucleotides in length.

44. The plurality of guide RNA template nucleic acids of 43, wherein the sequence common to the plurality of template nucleic acids of (c) (ii) comprises a scaffold sequence that can bind to a CRISPR-Cas effector protein.

45. The plurality of guide RNA template nucleic acids of 43 or 44, wherein the plurality comprises 3 to 20,000 of said template nucleic acids.

46. The plurality of guide RNA template nucleic acids of any one of 43-45, wherein the guide sequences encoded in the template nucleic acids hybridize to a target sequence within a genomic region of interest 500 to 2,000,000 contiguous nucleotides in length.

47. The plurality of guide RNA template nucleic acids of any one of 43-46, wherein said guide RNAs target sequences selected from a list of chromatin accessible target sequences generated according to the method of any one of 1-7.

48. The plurality of guide RNA template nucleic acids of any one of 43-47, wherein the template nucleic acids are linear single-stranded oligonucleotides.

49. A method of preparing a plurality of CRISPR-Cas guide RNAs for targeting a contiguous genomic region, the method comprising:
(a) contacting a plurality of template nucleic acids according to any one of 43-48, or a subset thereof, with an RNA polymerase; and
(b) transcribing the plurality of template nucleic acids, or a subset thereof, to produce a plurality of in vitro transcribed CRISPR-Cas guide RNAs for targeting a contiguous genomic region.

50. The method of 49, wherein the method further comprises, prior to said contacting of (a): contacting the template nucleic acid composition with a forward primer and a reverse primer to amplify the plurality of template nucleic acids, or a subset thereof.

51. The method of 50, wherein the forward primer is complementary to the first primer binding domain, and wherein the reverse primer is complementary to a sequence common to the plurality of template nucleic acids.

52. The method of any one of 49-51, wherein the method further comprises labeling the plurality of in vitro transcribed CRISPR-Cas guide RNAs according to the methods of any one of 35-40.

53. The method of any one of 49-52, wherein the method further comprises, after said transcribing of (b): contacting the plurality of in vitro transcribed CRISPR-Cas guide RNAs with a CRISPR-Cas effector protein to produce a plurality of CRISP-Cas effector complexes.

54. The method of 53, wherein the CRISPR-Cas effector protein is a Class 2 CRISPR-Cas effector protein.

55. The method of 54, wherein the Class 2 CRISPR-Cas effector protein is a Type II or Type V CRISPR-Cas effector protein.

56. A method of guiding a plurality of CRISPR-Cas effector complexes to a contiguous genomic region of interest, the method comprising contacting a contiguous genomic region in a target cell with a plurality of CRISPR-Cas effector complexes, wherein each CRISPR-Cas effector complex in the plurality of complexes comprises:
(a) a CRISPR-Cas effector protein;
(b) an in vitro transcribed guide RNA that hybridizes to a target sequence within the contiguous genomic region, thereby guiding the CRISPR-Cas effector complex to the contiguous genomic region,
wherein the plurality comprises 3 or more of said guide RNAs, and wherein the genomic region of interest is at least 500 contiguous nucleotides in length.

57. The method of 56, wherein the plurality comprises 3 to 20,000 of said guide RNAs, and wherein the guide RNAs to a target sequence within a genomic region of interest 500 to 2,000,000 contiguous nucleotides in length.

58. The method of 56 or 57, wherein the genomic region of interest is a non-repetitive genomic region.

59. The method of any one of 56-58, wherein the method comprises imaging, perturbing, altering, and/or regulating the genomic region of interest.

60. The method of any one of 56-59, wherein said guide RNAs target a target sequence selected from a list of chromatin accessible target sequences generated according to the method of any one of 1-7.

61. The method of any one of 56-60, wherein said guide RNAs are labeled according to the methods of any one of 35-40.

62. The method of any one of 56-61, wherein said CRISPR-Cas effector protein is a Class 2 CRISPR-Cas effector protein.

63. The method of 62, wherein the Class 2 CRISPR-Cas effector protein is Type II or Type V CRISPR-Cas effector protein.

64. The methods of any one of 56-63, wherein the CRISPR-Cas effector protein is catalytically inactive or has nickase activity.

65. The methods of any one of 56-64, wherein the CRISPR-Cas effector protein comprises a heterologous protein.

66. The method of 65, wherein the heterologous protein is a transcription repressor, a transcription activator, a base editor, a reverse transcriptase, or an epigenetic modifier.

67. The methods of any one of 56-66, wherein the target cell is a eukaryotic cell.

68. The method of 67, wherein the eukaryotic cell is a mammalian cell.

69. The method of 68, wherein the mammalian cell is a human cell.

70. The method of any one of 56-69, wherein the cell is in vitro.

71. The method of any one of 56-69, wherein the cell is ex vivo.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example 1: Robust High Resolution Dynamic Imaging of Chromatin DNA Communication using Oligo-LiveFISH

### Introduction

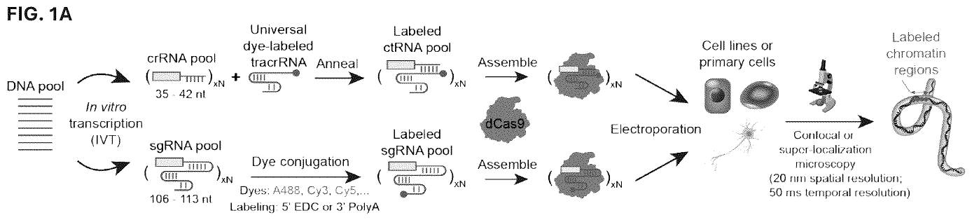

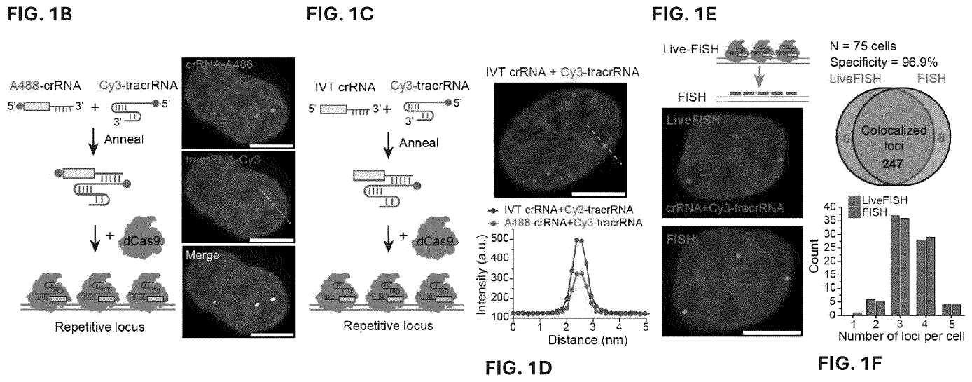

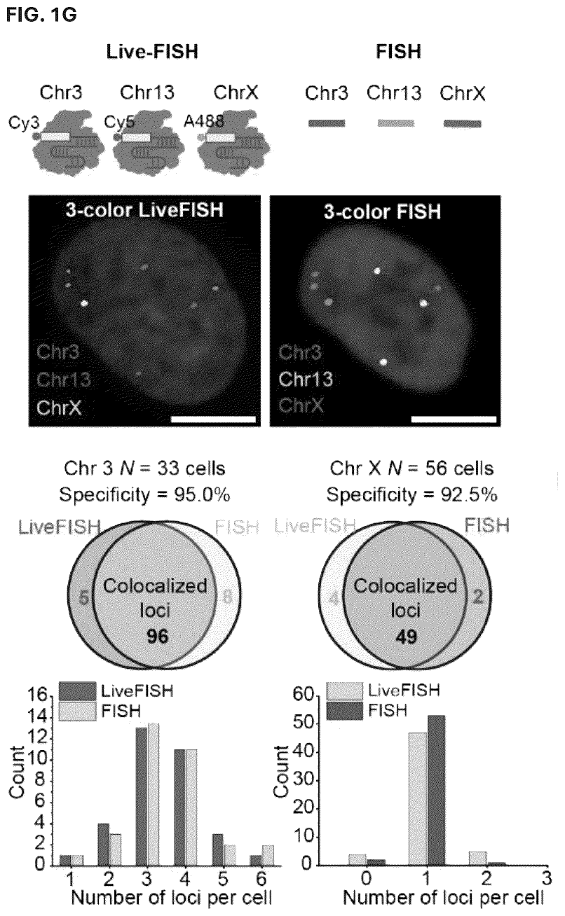

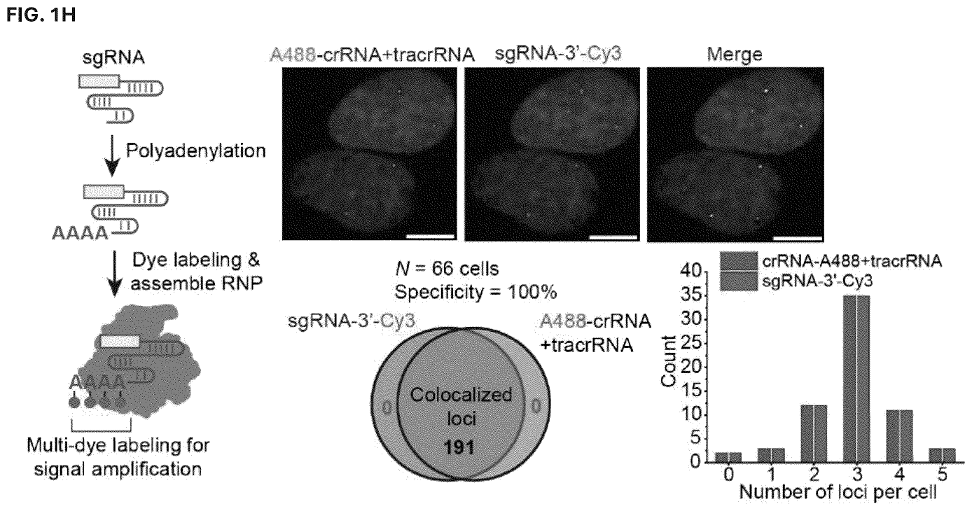

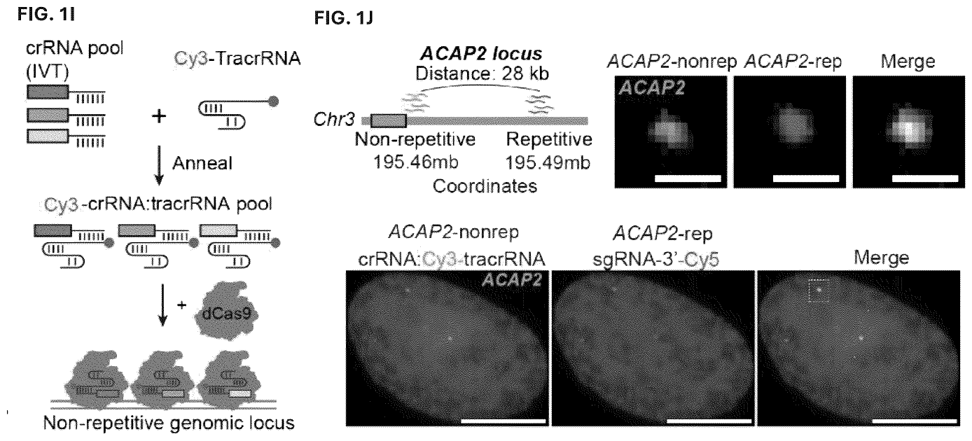

Here we report Oligo pool-based LiveFISH (Oligo-LiveFISH), a platform for flexibly imaging both repetitive and non-repetitive genomic loci and studying chromatin dynamics in live cells (FIG. 1A). We exploited approaches for designing effective gRNA oligo pools to achieve non-repetitive loci imaging by combining a pool of in vitro transcribed (IVT) crRNAs with a universal fluorescently labeled trans-acting crRNA (tracrRNA). We characterized key parameters affecting the SNR and established general principles for designing gRNAs. We further integrated Oligo-LiveFISH with 3D super-localization microscopy and tracked nonrepetitive regions with high spatial (20 nm) resolution and temporal (50 ms) resolution, revealing the highly sub-diffusive motion of chromatin loci consistent with fractional Brownian motion (fBM). Combining with dynamic modeling, we discovered two modes of chromatin communication: 1D cis-communication that dominates dynamic chromatin interaction at short distances (up to 300 kb), and 3D trans-communication playing a significant role at long distances (over one megabase). Oligo-LiveFISH imaging results are consistent with Hi-C sequencing data with further insights into dynamic chromatin communication at the single-cell level. Oligo-LiveFISH provides a robust and versatile method for visualizing diverse endogenous genomic loci in real-time across various cell types, which can be applied to study a variety of biological questions such as enhancer-promoter interactions in live cells.

### Results

Design of Oligo-LiveFISH and Optimization of gRNA Oligo Pools for Non-Repetitive Genomic Imaging

Oligo-LiveFISH is designed to utilize a pool of fluorescent ribonucleoproteins (fRNPs) for imaging. These fRNPs consist of three components: a nuclease-dead dCas9 protein, a CRISPR-associated crRNA containing a complementary sequence to the target DNA, and a universal trans-acting crRNA (tracrRNA) that pairs with the crRNA to form the gRNA structure recognized by dCas9. To systematically compare the efficacy of using crRNA: tracrRNA duplex with a single guide RNA (sgRNA), we also designed fRNPs containing dCas9 and fluorescently labeled sgRNAs (Table 1).

To flexibly image non-repetitive genomic regions, we assemble a pool of fRNPs with distinct crRNA sequences to tile the region of interest (FIG. 1A, Table 1-2). Briefly, a mixed single-stranded DNA oligo pool encoding crRNA or sgRNA was chemically synthesized, amplified to generate a double-stranded DNA pool, and in vitro transcribed (IVT) to produce a mixed crRNA pool or an sgRNA pool. The crRNA pool was further annealed with a universal dye-labeled tracrRNA to form crRNA: tracrRNA duplexes. For the sgRNA pool, we chemically conjugated it with a dye of choice. The purified, dye-labeled crRNA: tracrRNA or sgRNA pools were then assembled with purified dCas9 protein as fRNPs and electroporated into live cells.

The previous LiveFISH system used dye-labeled crRNA annealed with an unlabeled tracrRNA to target a repetitive region. Here we employed a universal, fluorescently labeled tracrRNA (5′-Cy3-tracrRNA) to avoid dye labeling of the crRNA pool. To compare these approaches, we annealed 5′-Cy3-tracrRNA and 5′-A488-crRNA targeting a 29-kb repetitive genomic region containing 529 copies of repeats on chromosome 3 (Chr3q29)near the ACAP2 gene in human U2OS osteosarcoma cells (ACAP2-rep). The A488-crRNA signal colocalized with the Cy3-tracrRNA signal (FIG. 1B), confirming that dye labeling preserved the secondary structure of tracrRNA for annealing and assemble with dCas9. We further compared the imaging quality using in-house in vitro transcribed crRNA and commercially synthesized dye-labeled crRNA and observed comparable signal-to-noise ratio (SNR) using both approaches under confocal microscope (FIG. 1C-D, microscope B in six microscope setups used in this study).

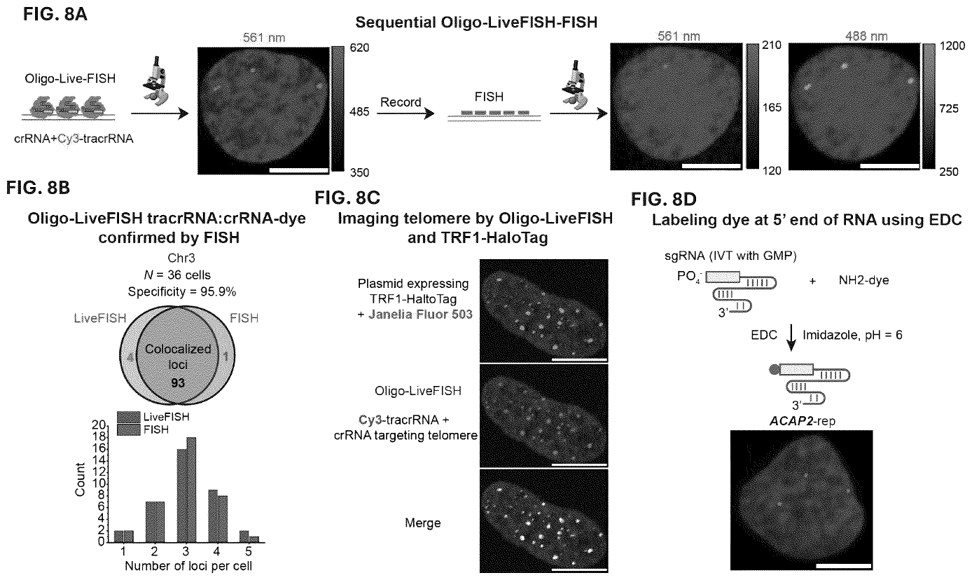

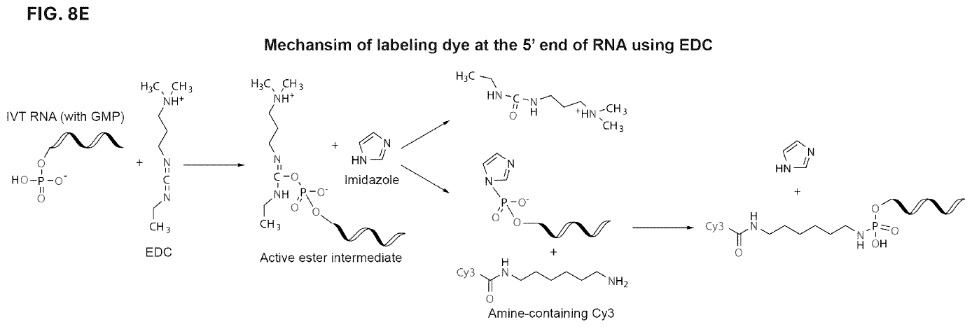

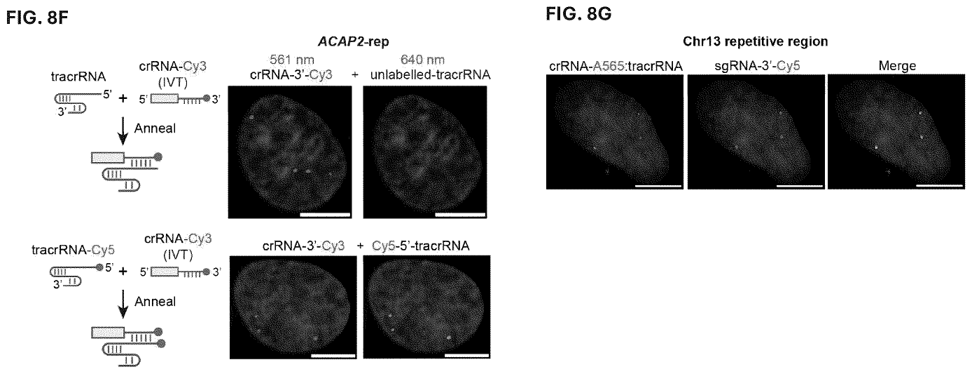

To confirm Oligo-LiveFISH targeting specificity, we validated it with standard FISH. Since the harsh conditions of FISH protocols disrupt Oligo-LiveFISH signals, we developed a sequential Oligo-LiveFISH-FISH imaging approach (FIG. 8A, See Methods). The FISH probe was designed to target a neighboring region of the Oligo-LiveFISH target (Table 2) and labeled with a different dye to ensure that the FISH signal was not residual from the Oligo-LiveFISH signals. The FISH signal colocalized with the Oligo-LiveFISH signal, confirming the imaging specificity of Oligo-LiveFISH, using either dye-labeled tracrRNA (FIG. 1E-F) or dye-labeled crRNA (FIG. 1G, FIG. 8B). Specificity was 96.9% for the ACAP 2-rep site using dye-labeled tracrRNA (FIG. 1F-G) and 95.9%, 95.0%, and 92.5% for Chr3 ACAP2-rep, Chr13, and ChrX repetitive sites using dye-labeled crRNA (FIG. 1G, FIG. 8B). This experiment also confirmed that Oligo-LiveFISH could be multiplexed to image multiple genomic loci simultaneously in living cells using a pool of compatible probes (FIG. 1G) 21. The specificity was further confirmed by colocalization of telomere signals labelled by Oligo-LiveFISH and the telomeric-binding protein TRF1-HaloTag expressed from a transfected plasmid (FIG. 8C).

Next, we investigated how RNA chemistry affected imaging efficiency. We tested three methods to label IVT RNA: (1) labeling the uridine nucleoside by incorporating Cy3- or Cy5-UTP with unlabeled UTP during RNA synthesis; (2) labeling the 5′-end by using the heterobifunctional, zero-length carbodiimide crosslinker EDC (1-ethyl-3-(3-(dimethylaminopropyl)-carbodiimide), imidazole, and amine-containing Cy3 (FIG. 8D-8E); or (3) labeling the 3′-end by using azido-modified adenosine nucleotides and yeast poly(A) polymerase³⁵ (FIG. 1H). Method (1) showed inefficient chromatin labeling, likely due to the disruption of RNA secondary structure by dye-UTP incorporation. In contrast, Methods (2) and (3) that label RNA at the termini achieved effective chromatin labeling. We primarily chose Method (3) for its simplicity and potential to add multiple dyes per RNA at the 3′ end. For example, crRNA labeled using Method (3) and annealed with unlabeled tracrRNA or 5′-Cy5-tracrRNA achieved robust and specific labeling of ACAP2-rep loci (FIG. 8F). Moreover, we in vitro transcribed sgRNA and labeled with dyes at the 3′ end using Method (3). Interestingly, two-color Oligo-LiveFISH experiments using dye-labeled crRNA: tracrRNA and sgRNA achieved 100% colocalization (FIG. 1H, FIG. 8G).

We expanded Oligo-LiveFISH to non-repetitive regions by first using repetitive region imaging as a reference for characterizing imaging efficiency and specificity. We designed a crRNA: tracrRNA library to target a non-repetitive region upstream of ACAP2, which we termed ACAP2-nonrep (FIG. 1I-J). Our integrated pipeline for designing and synthesizing the crRNA: tracrRNA pool included a computational algorithm that identifies all possible protospacer adjacent motif (PAM, NGG for the Cas9) sites in or adjacent to the target region and removes potential off-target guides ((FIG. 1I, see Methods). To synthesize crRNAs, we PCR amplified the synthesized DNA oligo library followed by IVT to generate a crRNA pool (FIG. 1I). This crRNA pool was annealed with a universal dye-labeled tracrRNA and mixed with dCas9 to assemble the fRNP pool. Using 24 Cy3-crRNA: tracrRNAs, we observed robust labeling of the ACAP 2-nonrep locus in live U2OS cells, which colocalized with the downstream ACAP2-rep locus co-imaged using a fRNP containing sgRNA-3′-Cy5, indicating high imaging specificity (FIG. 1J). While the repetitive region generally exhibited higher SNR than the non-repetitive region, our results demonstrated that Oligo-LiveFISH with fluorescent crRNA: tracrRNA pools can image non-repetitive genomic regions in live cells (data not shown).

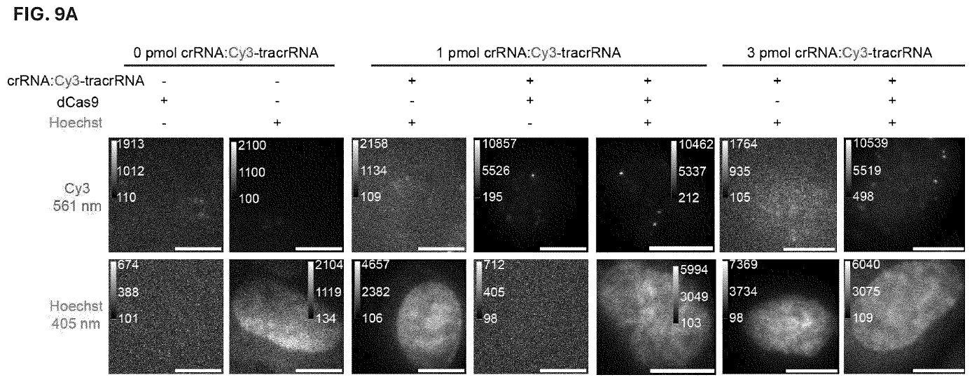

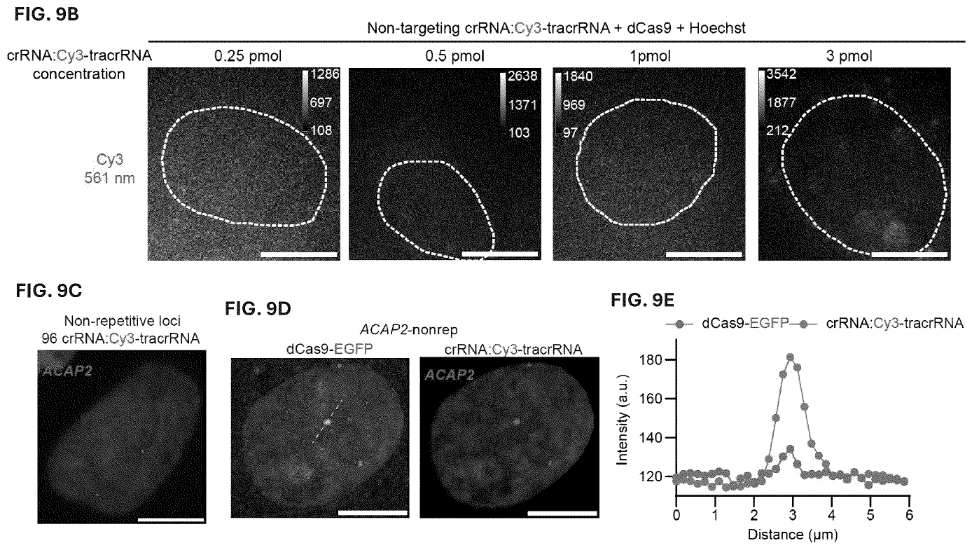

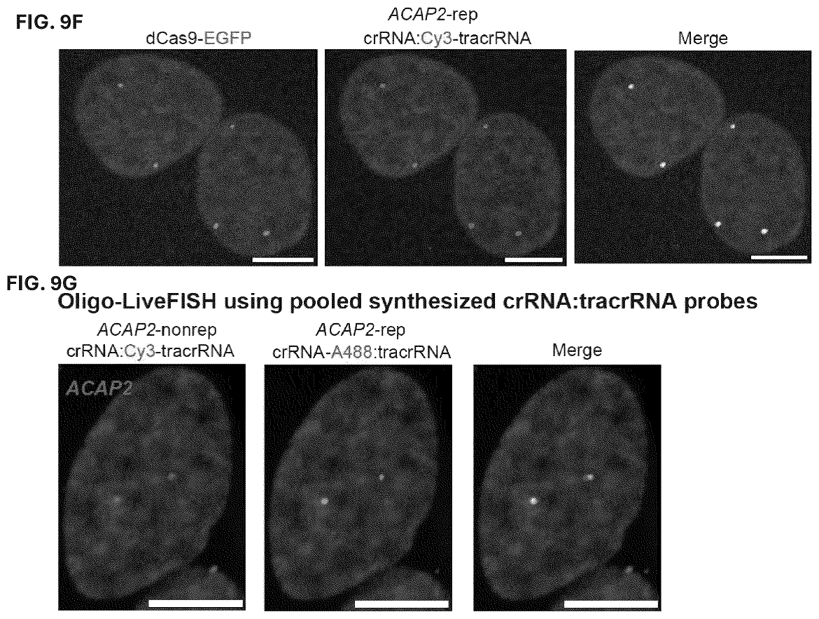

As controls, delivery of targeting dye-crRNA: tracrRNA without dCas9, dCas9 without dye-crRNA: tracrRNA, or non-targeting crRNA with dCas9 to U2OS cells did not exhibit observable signals (FIG. 9A,9B). It is possible that fRNPs targeting the repetitive region might decondense chromatin, facilitating non-repetitive fRNPs to access their target region. To rule out this possibility, we imaged the ACAP 2-nonrep region without co-delivering probes targeting ACAP2-rep region. Our results confirmed that non-repetitive probes can independently label chromatin (FIG. 9C). Specificity was further validated by the colocalization of crRNA: Cy3-tracrRNA and dCas9-EGFP in both non-repetitive and repetitive genomic imaging (FIG. 9D-9F). In addition, compared to dCas9-EGFP, dye-RNA achieved higher SNR (FIG. 9E) and was easier for multiplexing (FIG. 1G). Besides producing crRNA: tracrRNA from the multi-well plate, which provided flexibility in choosing specific subsets, we tested a more economical approach by synthesizing pooled DNA oligos and amplifying the entire library, which demonstrated efficient labeling (FIG. 9G).

TABLE 1
Sequence of templates, primers and crRNA/tracrRNA/sgRNA used in the study

A common template used for generating crRNA or sgRNA is the following:
Adapter + T7 promoter + crRNA/sgRNA spacer + crRNA/sgRNA common region
(1) crRNA oligo pool from Twist Bioscience (N is any nucleotide and varies
from 12-19 for oligo pools; the spacer is underlined)
Template:
Adapter +
TAATACGACTCACTATA + GNNNNNNNNNN + GTTTAAGAGCTATGCTGTT
TTG
Forward primer: Adapter sequence
Reverse primer: CAAAACAGCATAGCTCTTAAAC
(2) crRNA oligo pool from IDT (N is any nucleotide and varies from 12-19 for
oligo pools; the spacer is underlined)
Forward primer:
TAATACGACTCACTATA + GNNNNNNNNNN + GTTTAAGAGCTATGCTG
Reverse primer: CAAAACAGCATAGCTCTTAAAC
(3) sgRNA oligo pool from Twist Bioscience (N is any nucleotide and varies
from 12-19 for oligo pools; the spacer is underlined):
Template:
Adapter + TAATACGACTCACTATA + GNNNNNNNNNNNN + GTTTAAGAGCT
ATGCTGGAAACAGCATAGCAAGTTTAAATAAGGCTAGTCCGTTATCAACT
TGAAAAAGTGGCACCGAGTCGGTGCTTTTTTT
Forward primer: Adapter sequence
Reverse primer: AAAAAAAGCACCGACTCGGTGCCAC
(4) DNA template for sgRNA for repetitive region (N is any nucleotide; the
spacer is underlined):
Template:
GAAAT + TAATACGACTCACTATA + NNNNNNNNNNNN + GTTTAAGAGCTA
TGCTGGAAACAGCATAGC
AAGTTTAAATAAGGCTAGTCCGTTATCAACTTGAAAAAGTGGCACCGAG
TCGGTGCTTTTTTT
Forward primer:
GAAAT + TAATACGACTCACTATA + NNNNNNNNNNNN + GTTTAAGAGCTA
TGCTG
Reverse primer: AAAAAAAGCACCGACTCGGTGCCAC
(5) Adapter Sequences:
Adapter 1: CTTGGTCAGACGAGTGC
Adapter 2: TGGTACGGGAACAGCAC
Adapter 3: CTCACCGCTCTTGTAGC
Adapter 4: AAGACGGCTGAGCCATC
(6) Oligo-LiveFISH crRNA, tracrRNA and sgRNA sequences:
dCas9 tracrRNAs:
5'-
GGAACCAUUCAAAACAGCAUAGCAAGUUUAAAUAAGGCUAGUCCGUUA
UCAACUUGAAAAAGUGGCACCGAGUCGGUGCUUUUUUU-3'
dCas9 crRNAs (N is any nucleotide and varies from 13-20 for oligo pools; the
spacer is underlined):
5'-NNNNNNNNNNNNNGUUUAAGAGCUAUGCUGUUUUG-3'
dCas9 sgRNAs (N is any nucleotide and varies from 13-20 for oligo pools; the
spacer is underlined):
5'-
NNNNNNNNNNNNNGUUUAAGAGCUAUGCUGGAAACAGCAUAGCAAGU
UUAAAUAAGGCUAGUCCGUUAUCAACUUGAAAAAGUGGCACCGAGUCG
GUGCUUUUUUU-3'

TABLE 2
Oligo-LiveFISH and FISH probes targeting repetitive regions

| | | | Oligo-LiveFISH | FISH probe | |
|---|---|---|---|---|---|
| crRNA/sg | | | probe position | position | |
| RNA | Spacer Seq | repeats | (hg38) | (hg38) | References |
| ChrX-rep | TAGAGTCTGCAT | x666 | ChrX: 26096- | ChrX: 10001- | This study |
| | | | 44821 | 44814 (WI2- | |
| | | | | 1786K17) | |
| ChrX- | Data not shown | non- | ChrX: 275569- | ChrX: 10001- | This study |
| PLCXD1- | | repetitive | 283797 | 44814 | |
| nonrep | | | (96 probes, IDT) | (WI2- | |
| | | | | 1786K17) | |
| Chr13-rep | ACCATTCCTTC | x358 | Chr13: 112277011- | Chr13: 11227 | Ma et al., |
| | | | 112319170 | 6804- | 201612 |
| | | | | 112319743 | |
| | | | | (WI2- | |
| | | | | 438L18) | |
| Chr13- | Data not shown | non- | Chr13: 112577342- | Chr13: 11227 | This study |
| TUBGCP 3 | | repetitive | 112588167 | 6804- | |
| | | | (96 probes, IDT) | 112319743 | |
| | | | | (WI2- | |
| | | | | 438L18) | |
| Chr3- | Data not shown | non- | Chr3: 195446441- | Chr3: 195434 | This study |
| ACAP2- | | repetitive | 195470879 | 695- | |
| nonrep | | | (192 probes, IDT) | 195587044 | |
| (site 1 | | | | (RP11- | |
| 195.5Mb) | | | | 165B7) | |
| Chr3- | Data not shown | non- | Chr3: 195458383- | Chr3: 195434 | This study |
| ACAP2- | | repetitive | 195478290 | 695- | |
| nonrep- | | | (136 probes, Twist | 195587044 | |
| site 1 | | | Bioscience) | (RP11- | |
| 195.5Mb | | | | 165B7) | |
| Chr3- | TGATATCACAG | x529 | Chr3: 195478317- | Chr3: 195434 | Ma et al., |
| ACAP2- | | | 195506985 | 695- | 201612 |
| rep- | | | | 195587044 | |
| site2 | | | | (RP11- | |
| 195.5Mb | | | | 165B7) | |
| Chr3-site3 | AGGTATGGGTG | x116 | Chr3: 195775389- | N.A. | Gao et al., |
| 195.7Mb | | | 195777939 | | 202113 |
| Chr3-site4 | TGGACAGTGGGA | x133 | Chr3: 198098870- | N.A. | This study |
| 198.1Mb | | | 198105828 | | |
| MUC4 | Data not shown | non- | Chr3: 195808706- | N.A. | This study |
| gene | | repetitive | 195815721 (96 | | |
| | | | probes, IDT) | | |
| FOS gene | Data not shown | non- | Chr14: 75238783- | Chr14: 75161 | This study |
| promoter | | repetitive | 75278649 (237 | 753 | |
| | | | probes, Twist | -75339259 | |
| | | | Bioscience) | (787H13) | |
| FOS gene | Data not shown | non- | Chr14: 75044178- | Chr14: 75161 | This study |
| enhancer | | repetitive | 75084248 (343 | 753- | |
| | | | probes, Twist | 75339259 | |
| | | | Bioscience) | (787H13) | |
| MYC gene | Data not shown | non- | Chr8: 127696155- | N.A. | This study |
| promoter | | repetitive | 127736034 (340 | | |
| | | | probes, Twist | | |
| | | | Bioscience) | | |
| MYC gene | Data not shown | non- | Chr8: 127650882- | N.A. | This study |
| enhancer | | repetitive | 127691147 (375 | | |
| | | | probes, Twist | | |
| | | | Bioscience) | | |
| Telomere | GUUAGGGUUAGG | N.A. | N.A. | N.A. | Chen et |
| | GUUAGGGUUA | | | | al., 201314 |
| Non- | GAACGACTAGTTA | N.A. | N.A. | N.A. | |
| targeting | GGCGTGTA | | | | |

### Characterization and Optimization of Oligo-LiveFISH for Non-Repetitive Loci Imaging

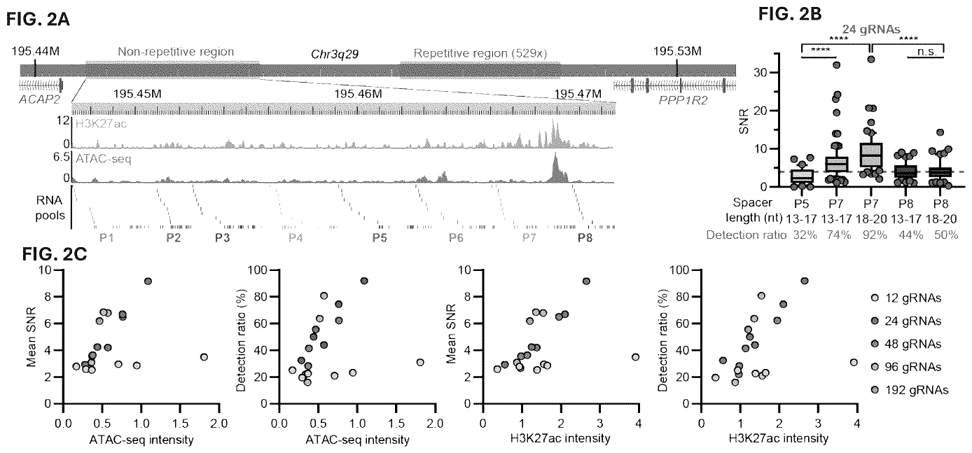

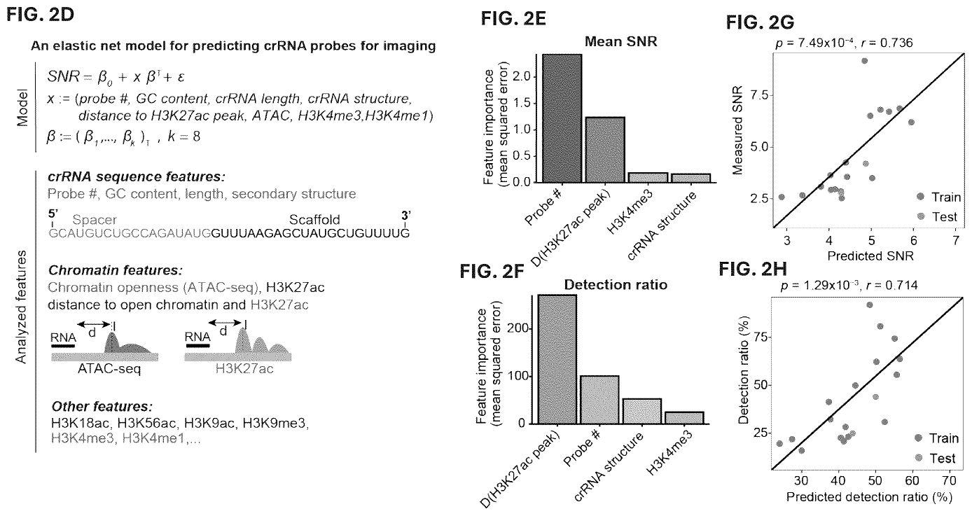

To further optimize Oligo-LiveFISH, we systematically characterized how imaging efficiency depends on: (1) the number of distinct crRNA: tracrRNA probes tiling a non-repetitive region; (2) biochemical crRNA features such as spacer length, GC content, and secondary structure; and (3) the accessibility and openness of the target chromatin region (FIG. 2A). We developed a method to calculate SNR specifically for punctate signals such as DNA loci (see Methods). Utilizing this enhanced SNR metric, we quantified the dependence of imaging efficiency on the crRNA: tracrRNA and various chromatin features.

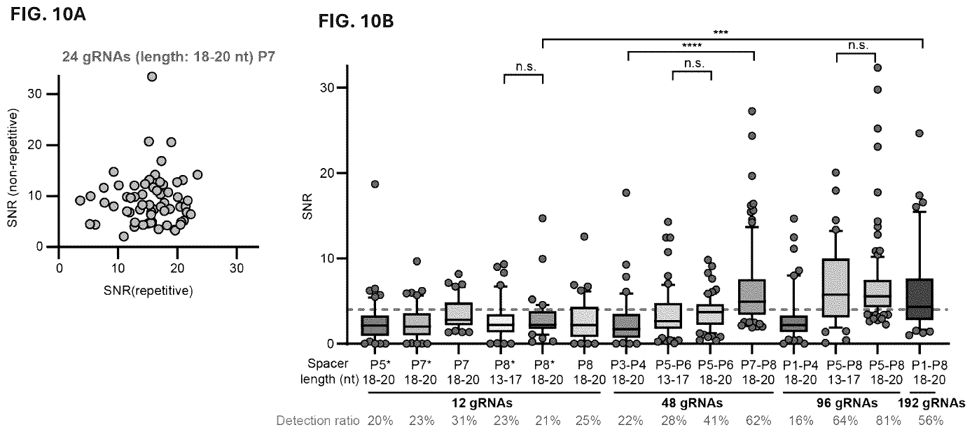

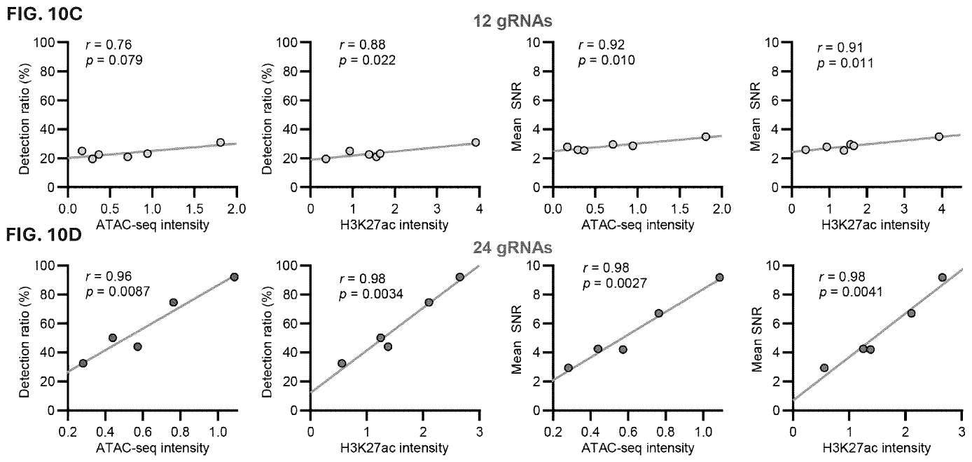

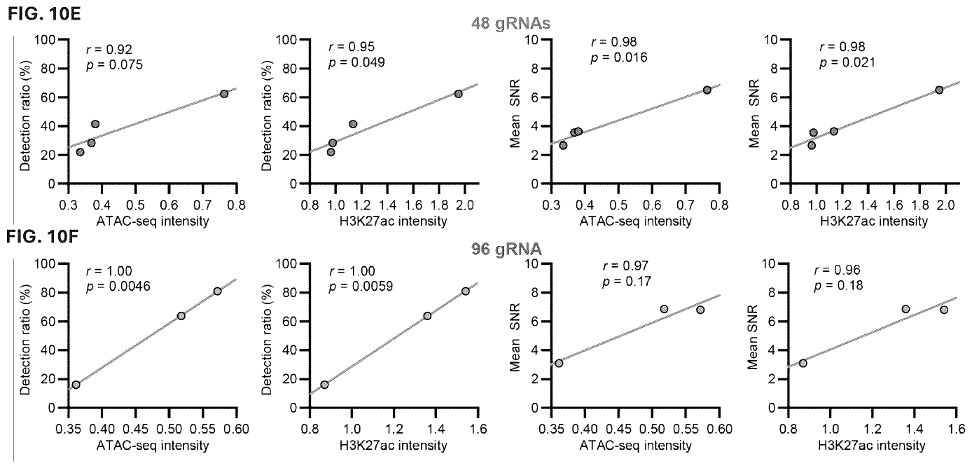

To determine the optimal number of probes, we designed 192 unique crRNA: tracrRNAs divided into 8 subgroups (P1-P8) to tile the ACAP2-nonrep region, with each subgroup containing 24 crRNA: tracrRNAs (FIG. 2A). We tested combinations of subgroups containing 12, 24, 48, 96 and 192 crRNA: tracrRNAs for imaging. To test the effect of crRNA length, we also designed two sets of probes for each subgroup: one with a longer spacer length (18-20 nucleotide (nt)) and the other with a shorter spacer length (13-17 nt). These crRNA: tracrRNAs targeting the non-repetitive region were co-delivered with sgRNAs with a different dye targeting the repetitive region, which served as a reference to calculate SNR (FIG. 10A). The measured SNRs using 24 crRNA: tracrRNAs are shown in FIG. 2B and SNRs using other numbers of crRNA: tracrRNAs are shown in FIG. 10B. We also calculated a ‘detection ratio’, defined as the number of DNA loci with measured SNR above 4 divided by the total number of loci. The results showed that as few as 24 crRNA: tracrRNAs were able to generate detectable signals (FIG. 2B). Interestingly, the mean SNR and detection ratio in each subgroup correlated positively with the mean intensity of H3K27ac and ATAC-seq signals at the targeted sites, indicating that chromatin accessibility influences imaging efficiency (FIG. 2C, FIG. 10C-10F). We next investigated this correlation using machine learning.

Machine Learning to Investigate the Key Parameters that Determine the Imaging Efficiency

We developed a machine learning model based on an elastic-net regularized generalized linear model to identify the determinant features of Oligo-LiveFISH imaging efficiency. We split 19 samples into a training set (17 samples) for model training and a test set (2 samples) for model evaluation. We examined 15 features, including the number of RNA probes, the GC content, spacer length, secondary structure of crRNAs and annealed crRNA: tracrRNAs, the distance of the target region to the closest H3K27ac peak and ATAC peak, and a variety of chromatin features such as the intensity of ATAC-seq, H3K27ac, H3K4mel, H3K18ac, H3K56ac, H3K9ac, H3K9me3, and H3K4me3 (FIG. 2D).

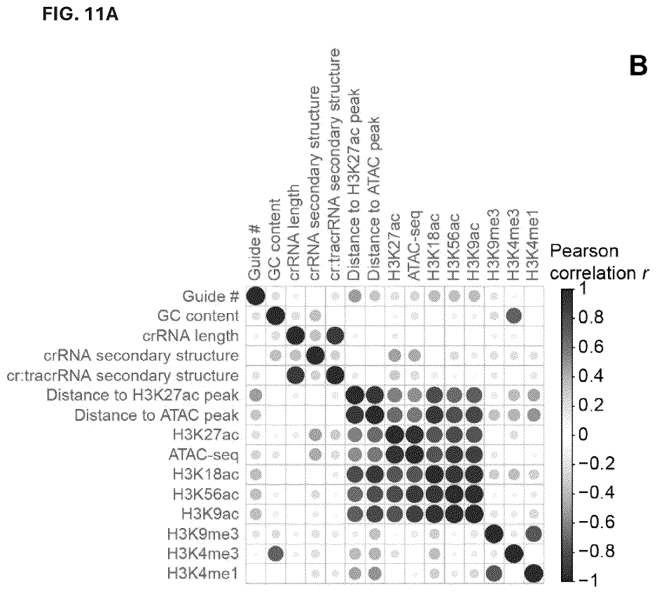

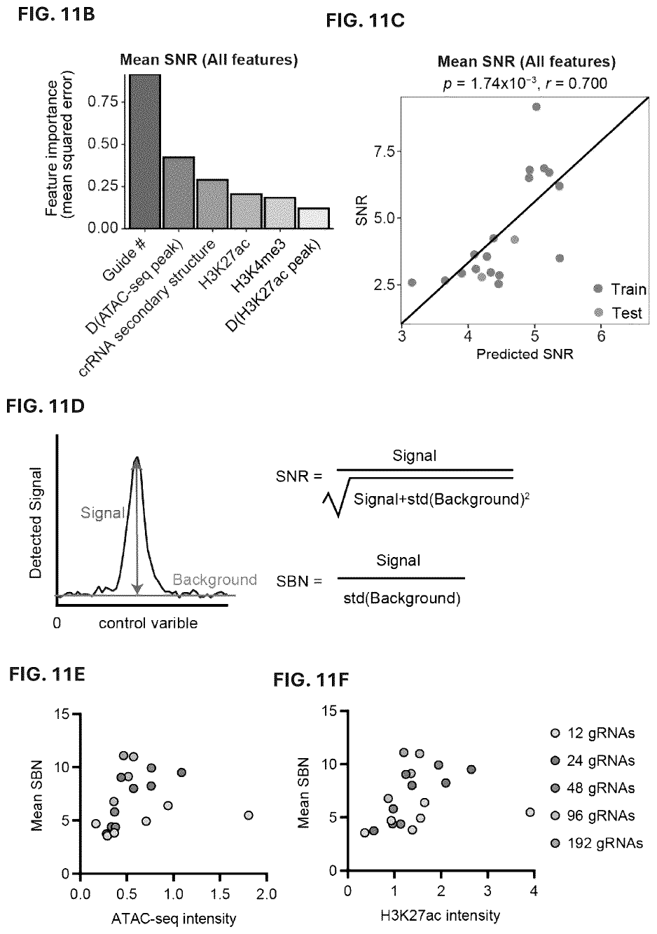

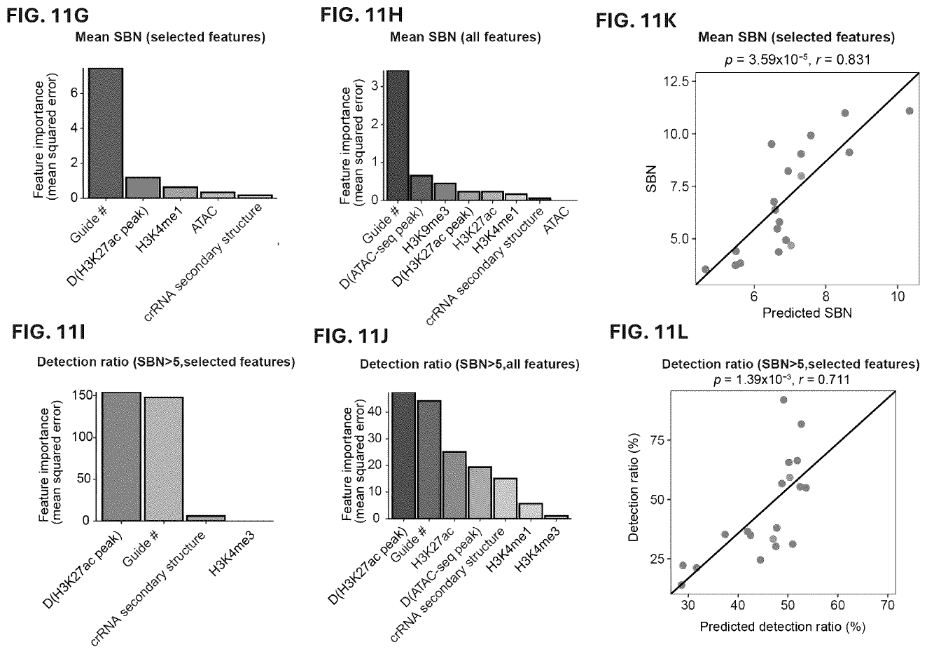

We first performed correlation analysis to remove correlated features and selected 8 features as variables for the machine learning model (FIG. 11A). Interestingly, our analysis revealed that the most critical features for achieving higher SNR (FIG. 2E) and detection ratio (FIG. 2F) are the number of gRNA species and chromatin accessibility (distance to the nearest H3K27ac or ATAC peak). The predicted SNR and detection ratio correlated well with experimental results (SNR: R=0.736, p value=7.49×10⁻⁴, FIG. 2G; detection ratio: R=0.714, p value=1.29×10⁻³, FIG. 2H). For comparison, we also trained the model using all 15 features without removing correlative features. Although the performance (mean squared error, m.s.e.=28.691) was less robust compared to using the 8 selected features (m.s.e.=20.739), it still indicated that gRNA number and chromatin accessibility are the most significant features in determining the imaging efficiency (FIG. 11B-11C).

Moreover, we calculated the signal-to-background-noise ratio (SBN, FIG. 11D, see Methods), a measurement often misconstrued in the community as SNR, and tested different thresholds to define the detection ratio. The results consistently indicated that the probe number and chromatin accessibility are the most critical features (FIG. 11E-11L). Additionally, we observed significant differences in SNR when using probes targeting chromatin of varying accessibility but no significant difference when using crRNAs of different lengths for the same probe set (FIG. 2B, 10B). This finding is consistent with the machine learning modeling results, highlighting that chromatin accessibility as a more important determinant than crRNA length. Therefore, to better image non-repetitive regions, we designed crRNA probes near the ATAC-seq and H3K27ac peaks for subsequent experiments. The principles we generated here for genome imaging could also be beneficial for designing gRNA pools in CRISPR-based gene perturbation.

### Oligo-LiveFISH Enables Robust Imaging of Various Non-Repetitive Genomic Regions in Diverse Cell Types

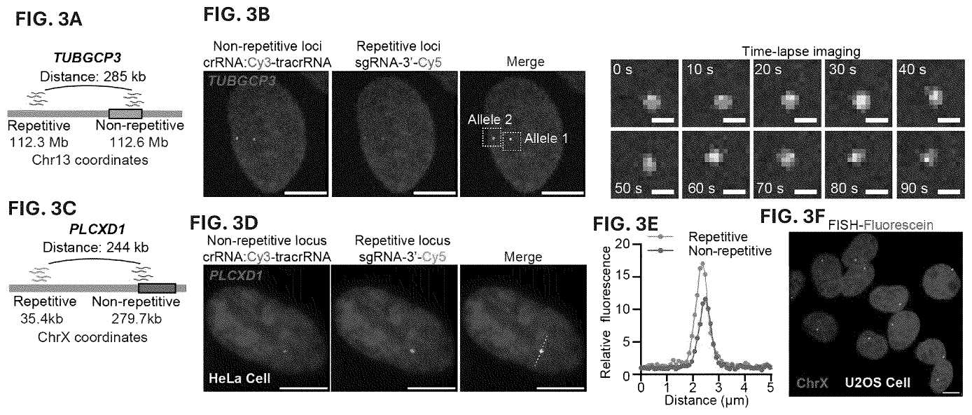

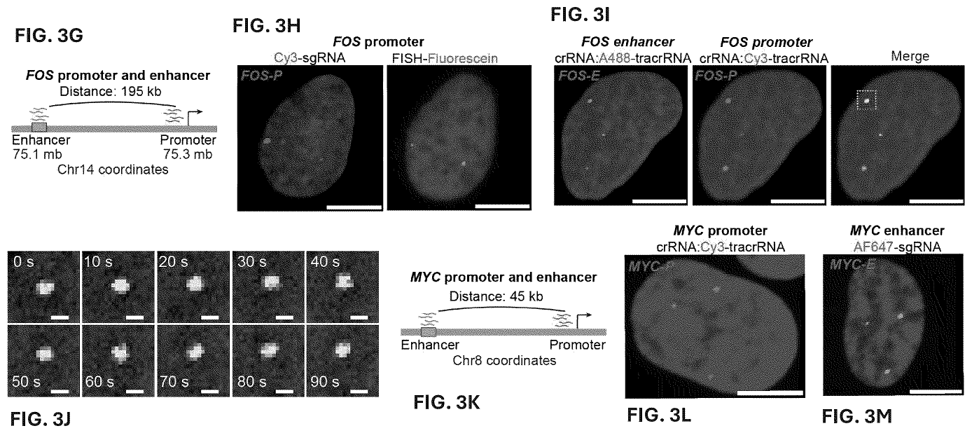

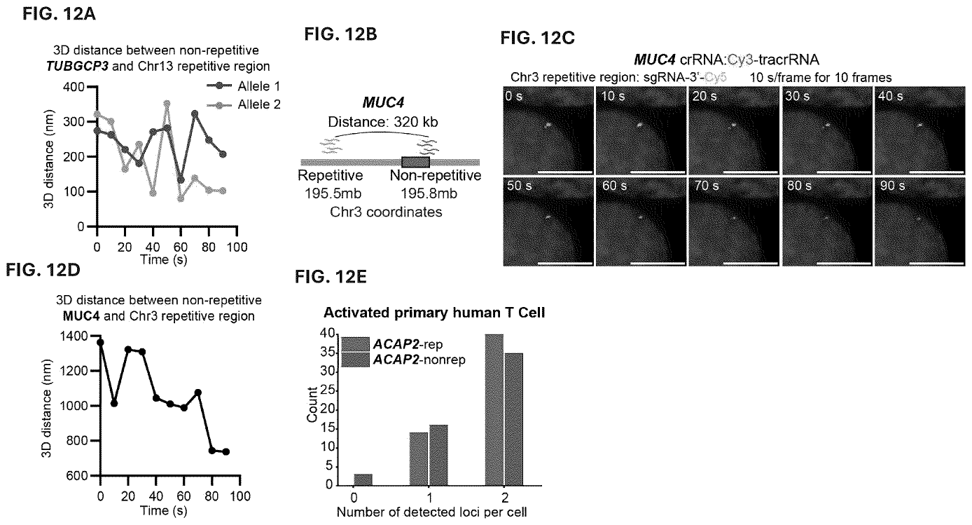

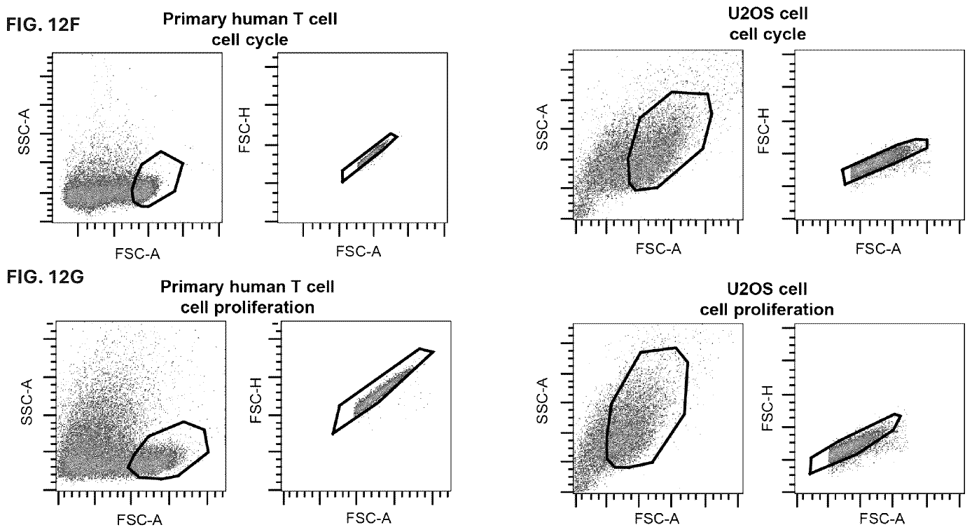

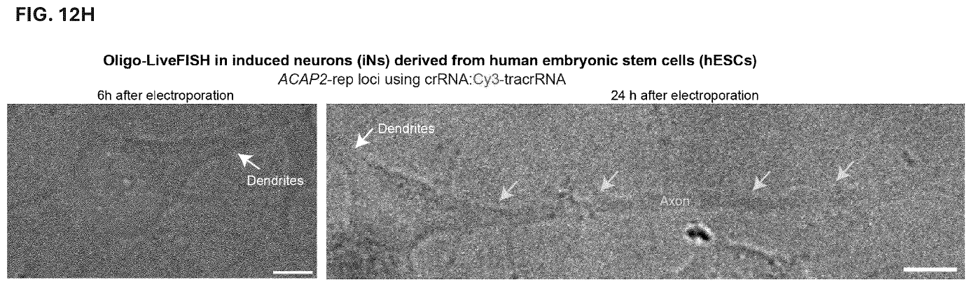

To test the generality of Oligo-LiveFISH, we applied it to visualize other non-repetitive genomic loci on different chromosomes. Since the repetitive regions on Chr13 and ChrX have been confirmed by FISH (FIG. 1G), we designed probes to image non-repetitive regions on Chr13 and ChrX downstream of these repetitive regions, which targets TUBGCP3 gene (Chr13, FIG. 3A-B) and PLCXD1 gene (ChrX, FIG. 3C-E), respectively, following the gRNA design principles summarized above. Our results demonstrated robust and specific imaging of these non-repetitive regions in U2OS cells and HeLa cells. We tracked both repetitive and non-repetitive TUBGCP 3 sites (~285 kb apart) with 10 s per frame for 10 frames and observed the distance between the two loci varied between 100 nm and 300 nm (FIG. 12A). This measurement uses the confocal microscope B, with the times of observation being 10 s apart. Therefore, we call this time-lapse mode, to distinguish with the case of continuous observation of time dependence with a wide-field microscope later in this paper.

Moreover, we imaged the MUC4 gene non-repetitive region and its upstream repetitive region (~320 kb apart) on Chr3, where the 3D distance fluctuated around 1000 nm (FIG. 12B-12D), indicating a high variability of chromosomal spatial organization across different chromosome sites. A comprehensive biophysical analysis of 3D dynamic spatial distance and genomic distance will be discussed in next sections. Notably, most cells showed only one ChrX locus in both U20S cells and HeLa cells using Oligo-LiveFISH (FIG. 3D). The observation is consistent with our FISH results (FIG. 1G, FIG. 3F) and the monosomic nature of ChrX in these cell lines revealed by karyotype, indicating one copy of chromosome X is lost. The specificity (FIG. 1E-G, FIG. 3F), multiplexing ability for different chromosomes (FIG. 1G, FIG. 3A-E), and compatibility with primary cells (vide infra), demonstrate the potential of Oligo-LiveFISH for cytogenetic diagnosis, such as preimplantation genetic testing for aneuploidy screening (PGT-A) in living cells.

We next imaged the enhancer and promoter of FOS, a gene critical for modulating the immediate early response in the neuronal activity and a trans-acting factor capable of stimulating gene expression for cell proliferation, differentiation, and survival in various cancers (FIG. 3G). Furthermore, recent studies revealed potential re-organization between its enhancer and promoter via time-course 5C mapping. We imaged the FOS promoter using dye-crRNA: tracrRNA and dye-sgRNA, with both methods showing robust labeling and specificity as confirmed by FISH (FIG. 3H). Moreover, we applied Oligo-LiveFISH to simultaneously label and track the dynamic movement of FOS promoter and enhancer (FIG. 3I-J). Similarly, we applied Oligo-LiveFISH to image the promoter and enhancer of another oncogene MYC in U2OS cells (FIG. 3K-M). These results suggested that Oligo-LiveFISH can capture the real-time dynamics of enhancer-promoter distance for a variety of genes.

Oligo-LiveFISH Enables Imaging of Non-Repetitive Genomic Regions in Primary Human T Cells and Human Embryonic Stem Cells (hESCs)-Induced Neurons (iNs)

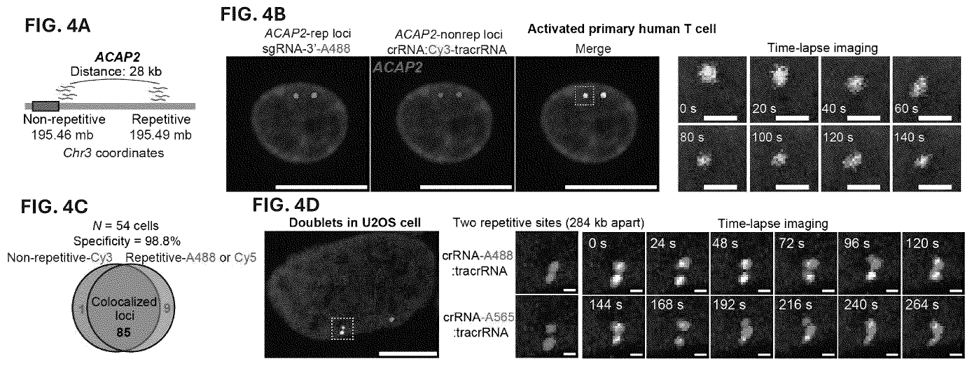

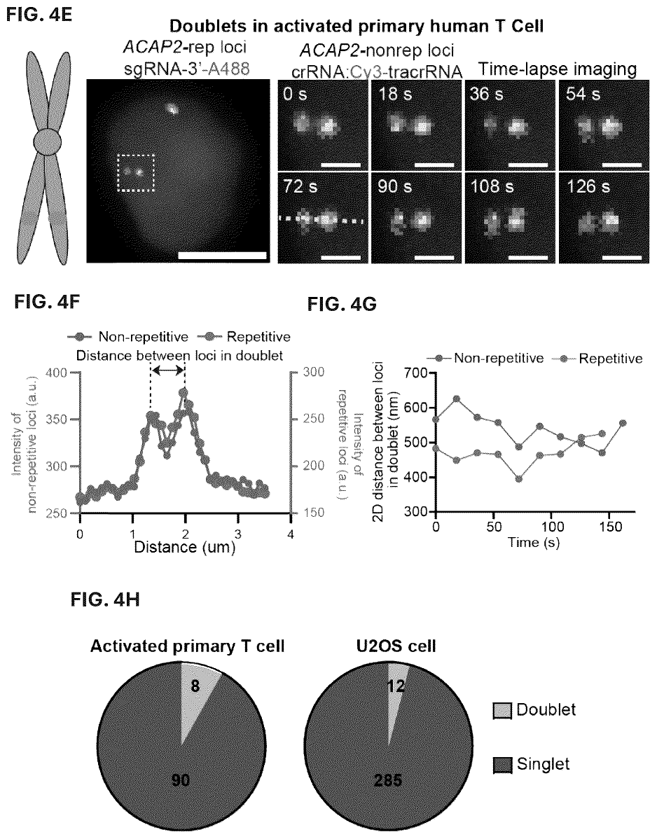

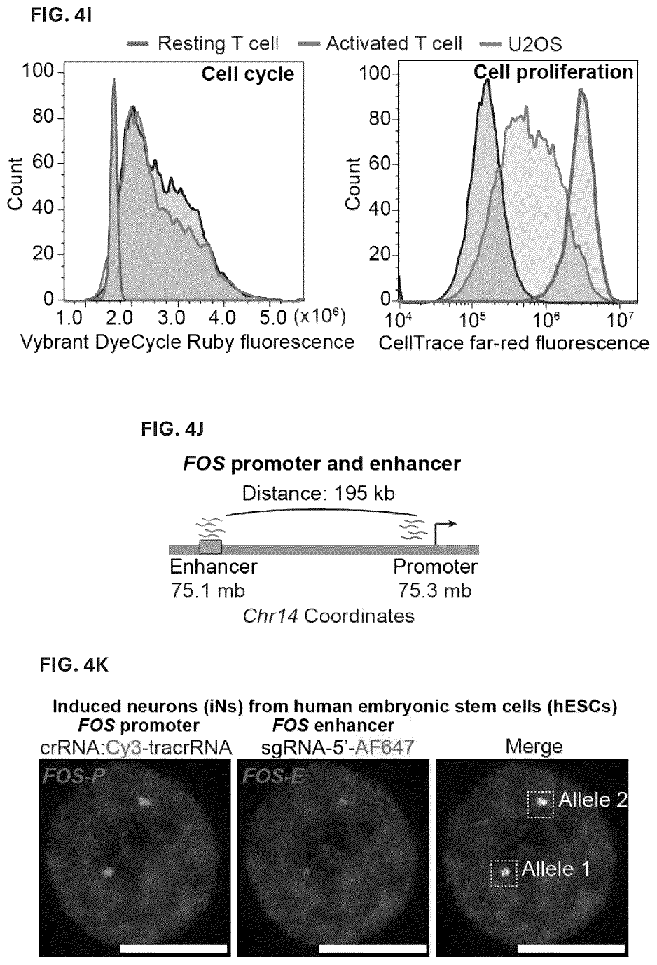

One unique advantage of Oligo-LiveFISH compared to other CRISPR-based chromatin imaging methods is that it bypasses the need to construct stable cell lines and can thus be applied to primary cells. Indeed, we used Oligo-LiveFISH to label non-repetitive regions in activated primary human T cells from anonymous healthy blood donors. The round-shape, suspension primary T cells are more challenging to image compared to the adherent, flat U2OS and HeLa cells. We first optimized the Oligo-LiveFISH procedures specifically for imaging suspension cells (See Methods). Using this optimized platform, we applied Oligo-LiveFISH to image repetitive and non-repetitive ACAP2 genomic regions on Chr3 in primary human T cells (FIG. 4A-B), achieving 98.8% specificity in imaging ACAP2-nonrep regions when compared to the signal at the repetitive sites (FIG. 4C). Unlike the heterogeneous aneuploid U2OS cells (FIG. 1E-G, FIG. 3E) which showed a wide range of detected loci number for Chr3, we detected two loci in most of the diploid T cells (FIG. 12E).

Live cells undergo active chromatin replication, segregation, and cell division during imaging. Interestingly, in both U2OS cells (FIG. 4D) and primary human T cells (FIG. 4E-G), we found a small fraction of loci that contained two detected lobes rather than a single dot, which we call “doublet”. We hypothesized these doublet loci to be the same chromatin site on newly replicated sister chromatids. We calculated the 2D distance between the two sister chromatids for both ACAP2-rep and ACAP2-nonrep sites, which fluctuated around 500 nm for the cell shown (FIG. 4E-G). Approximately 8% of loci in activated primary human T cells and 4% of loci in U2OS cells were doublets (FIG. 4H), indicating that it is a rare, but non-negligible phenomenon. To confirm that cells were actively replicating, we performed flow cytometry to measure cell cycle and cell proliferation (FIG. 12F-12G). In the cell cycle assay, cells were stained with Vybrant DyeCycle Ruby Stain to indicate total DNA content. Activated primary T cells and U2OS cells showed similar fluorescence intensity, and both were higher than resting T cells (FIG. 4I), indicating these actively replicating cells possessed more total DNA content than the non-replicating resting T cells. In the cell proliferation assay, U2OS cells and primary human T cells were stained with CellTrace dye and cultured for the same period before analysis. Compared to the resting T cells, activated primary human T cells and U2OS cells have lower CellTrace intensity (FIG. 4I), indicating more cell division within the same period. These imaging-based and flow cytometry assays indicate that the doublets we observed are probably replicated sister chromatids, suggesting Oligo-LiveFISH could be a useful platform for studying dynamic chromatin replication and segregation.

We further applied Oligo-LiveFISH to track chromatin regions in human embryonic stem cells (hESCs)-derived induced neurons (iNs). We first imaged iNs 6 h and 24 h after delivering fRNP targeting ACAP 2-rep regions via electroporation. The cells showed labeled loci along with dendrites and elongating axons (FIG. 12H), indicating that Oligo-LiveFISH does not disrupt the neuronal differentiation process. Next, we simultaneously imaged the FOS promoter and enhancer in iNs (FIG. 4J-K) and captured both sites. Taken together, these results confirmed that Oligo-LiveFISH can capture chromatin dynamics in living primary T cells or hESCs derived neuronal cells, which was difficult to image using previous approaches.

### Development of 2D and 3D Super-Localization Oligo-LiveFISH (SL-Oligo-LiveFISH) for High Resolution Dynamic DNA Imaging

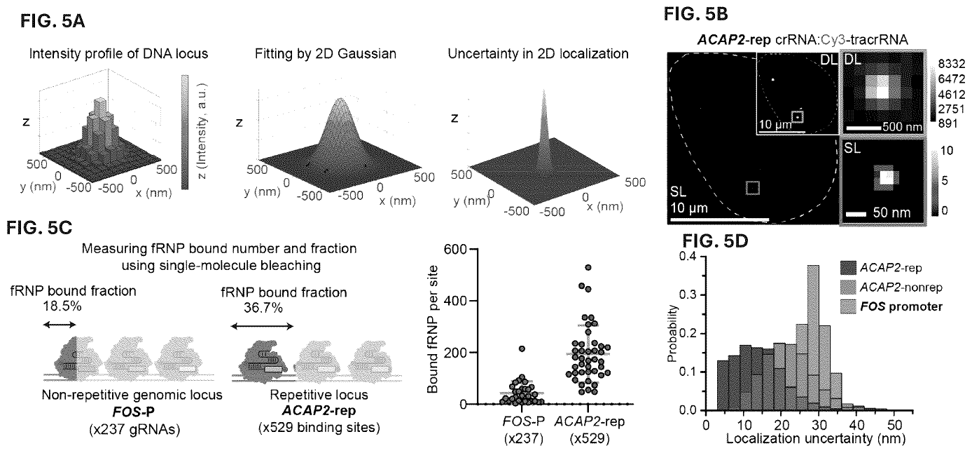

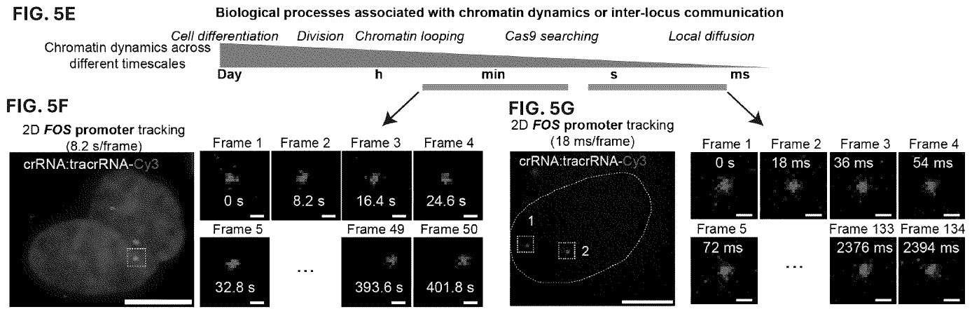

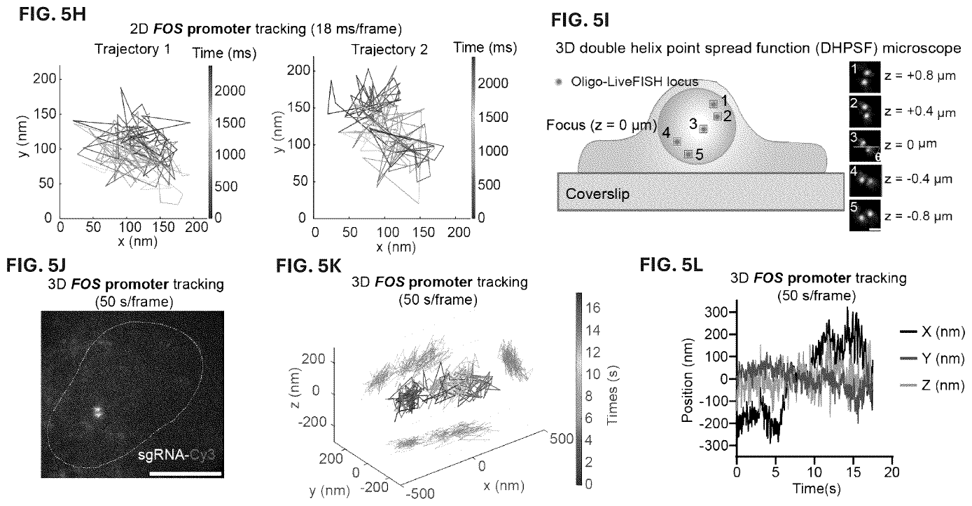

Having established that Oligo-LiveFISH is a valid technique for imaging genomic loci, we now show that the SNR values are large enough to enable detailed and quantitative 2D and 3D analysis. Extracting such information at a single-cell level provides microscopic detail which can lead to deeper biological and mechanistic insights. First, conventional light microscopy is fundamentally limited by diffraction limit (DL), preventing it from resolving closely spaced objects with distances below ~250 nm. However, for sparse emitters, by fitting the spot from a single small object (called the point-spread-function (PSF) of the microscope) with a suitable function such as a 2D Gaussian function, the localization uncertainty of the emitter can be much lower than the diffraction limit (FIG. 5A). This method is termed super-localization (SL) microscopy, which provides sub-pixel information about the position of the emitter, a method well-known in biophysical studies. Here we show that SL microscopy and Oligo-LiveFISH can be advantageously integrated (termed SL-Oligo-LiveFISH) to track a chromatin region with a localization precision of ~10-30 nm in both 2D and 3D. As shown in FIG. 5B, comparing the DL and SL images of the same ACAP2-rep site, the SL image provides much smaller (better) localization uncertainty. Note that the localization precision improves when more photons are recorded for each PSF.

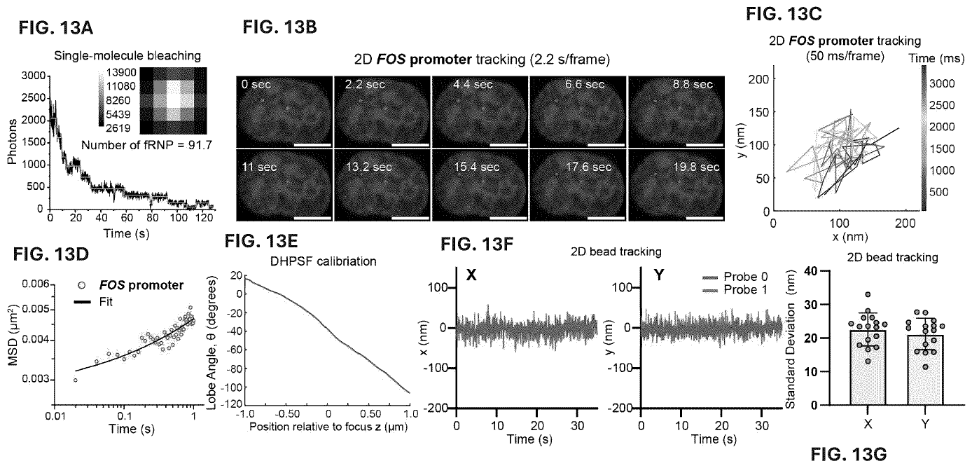

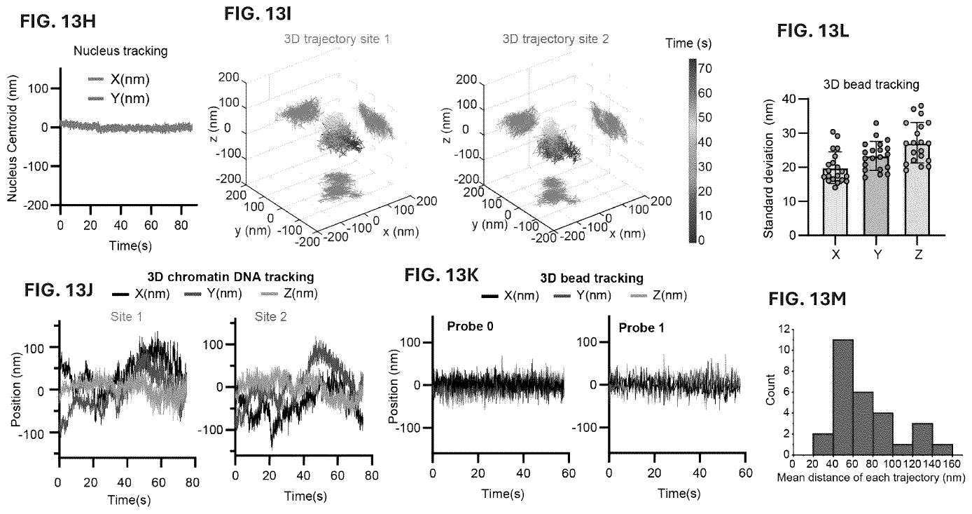

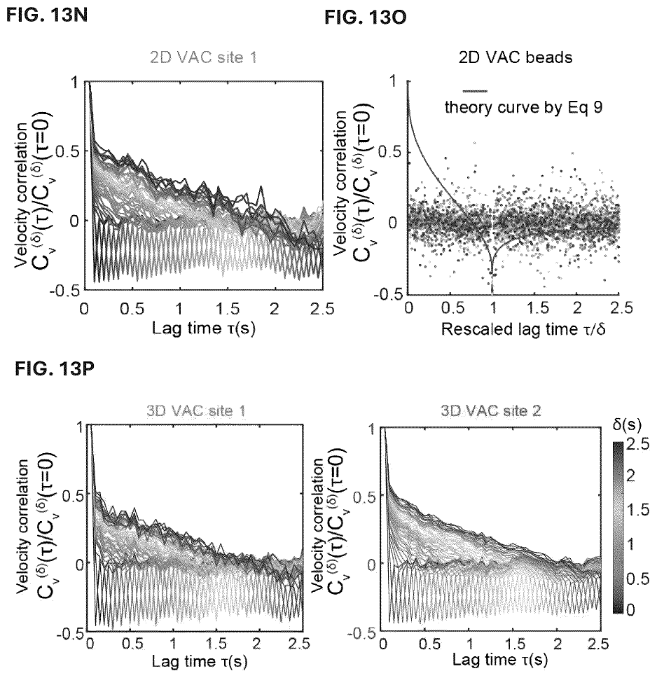

Since non-repetitive loci may have dimmer spots, we can quantify the number of bound fRNPs for each target site at equilibrium using bleaching of a labeled locus to the single-molecule level to calibrate (FIG. 13A, See Methods). For ACAP2-rep sites (529 repeats), there are 194±110 (mean±1S.D.) bound fRNPs for each locus (FIG. 5C). In contrast, when tiling the non-repetitive FOS promoter using 237 crRNA probes, there are only 44±44 (mean±1S.D.) bound fRNPs for each locus (FIG. 5C). Dividing by the total number of available binding sites, the bound fraction is 36.7% for the repetitive site but 18.5% for the non-repetitive site. This difference is due to the fact that for each specific probe, there is only one available binding site for the non-repetitive case. Accordingly, we observed higher localization uncertainty (FIG. 5D) for the non-repetitive genomic region (22.2±7.1 nm for ACAP2-nonrep site at 50 ms resolution and 29.0±3.4 nm for FOS promoter at 18 ms resolution) compared to the repetitive region (13.6±6.4 nm for ACAP2-rep site at 50 ms resolution). Importantly, despite larger (worse) localization uncertainty for the non-repetitive genomic region, the localization uncertainty in both cases is sufficient to allow analysis of long-distance enhancer-promoter looping interactions, which typically involve changes in distances of several hundred nanometers as reported previously. In the next sections, we show that this 20 nm precision allows deep investigation of the 2D and 3D spatial dynamics of various sites along the same chromosome, showing the unique utility of SL-Oligo-LiveFISH.

In addition to spatial resolution, unlike traditional FISH, temporal resolution is another important parameter to consider when tracking chromatin dynamics. Chromosomes are mechanical objects whose shape constantly changes in live cells during biological processes, which occur on time scales varying from milliseconds (such as local diffusion) to days (such as cell division and differentiation) (FIG. 5E). For example, in the next section, we show that the chromatin communication time between two sites 28 kb apart is as short as 0.44 s. Observing such fast dynamics necessitates a time resolution of ~50 ms or faster. However, faster temporal resolution sometimes requires shorter exposure times, at the price of lower spatial resolution. Without carefully considering and characterizing localization uncertainty, incorrect conclusions could be drawn when investigating chromatin dynamics and looping interactions.

Using 2D SL-Oligo-LiveFISH, we tracked the FOS promoter non-repetitive region at different time resolutions of 8.2 s (FIG. 5F), 2.2 s (FIG. 13B), 50 ms (FIG. 13C), and in the fastest case, 18 ms (FIG. 5G, 5H) per frame. Even in the fastest case of 18 ms per frame, the localization precision is 29.0±3.4 nm, which is sufficient for tracking chromatin dynamics in many rapid biological processes. A starting point for characterizing motion driven by thermal fluctuations is to calculate the mean-square displacement (MSD) curve as a function of time lag between positions (FIG. 13D). For this case we fit the MSD with the equation based on fractional Brownian motion (fBM) that considers both the dynamic and static localization error (Eq.5). We obtained an effective diffusion coefficient D*=(5.0±1.0)×10⁻⁴ μm²/s^{α}, subdiffusion exponent α=0.36±0.14, and localization error σ=27±1 nm for the FOS promoter (FIG. 13D), suggesting sub-diffusive behavior of FOS promoter at this timescale.

Extracting 2D dynamics from data in one z plane could potentially lose dynamical information because the DNA locus inherently moves in the 3D space, and it could potentially move out of the microscope focus during 2D data acquisition. Z-scanning confocal imaging attempts to solve this issue by acquiring multiple z slices at the cost of lower time resolution due to scanning speed limitations. To go further and extract fast DNA dynamics in 3D, we applied the double-helix point spread function (DHPSF) microscope (microscope C) to track the chromatin loci dynamics with high spatiotemporal resolution in 3D. This technique involves PSF engineering, in which the PSF of a microscope is modified optically to produce two spots, such that the angle of the two spots encodes the depth position of the source for 3D tracking (FIG. 5I, calibrated in FIG. 13E) over an axial range of 2 μm. This method allows simultaneous study of multiple loci with high resolution in x, y, and z and with high time resolution as shown below (FIG. 5J-L).

We applied 3D SL-Oligo-LiveFISH to track the non-repetitive FOS promoter using DHPSF at 50 ms per frame (FIG. 5J-L). Both 2D and 3D data indicated a subdiffusive behavior of FOS promoter on the timescale from tens of ms to several seconds, a timescale that has not been explored before for non-repetitive endogenous chromatin dynamics (FIG. 5F-L, FIG. 13B-13D).

We next used multiple microscopes (2D widefield, confocal, and 3D DHPSF) (1) to demonstrate that Oligo-LiveFISH is compatible with different microscope capabilities and (2) to confirm biophysical findings about chromatin dynamics with different imaging modalities.

### Two-Color Oligo-LiveFISH Tracking of Two Chromosome Sites and Biophysical Analysis in Single Cells

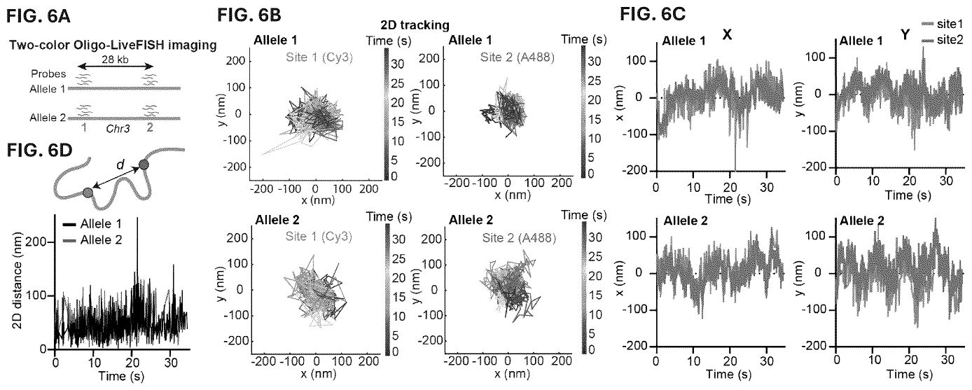

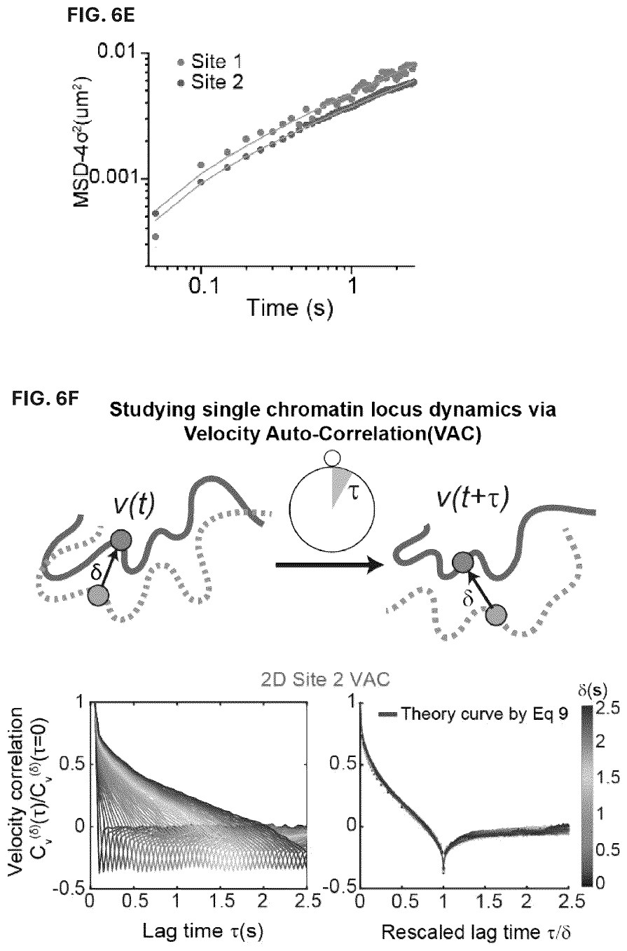

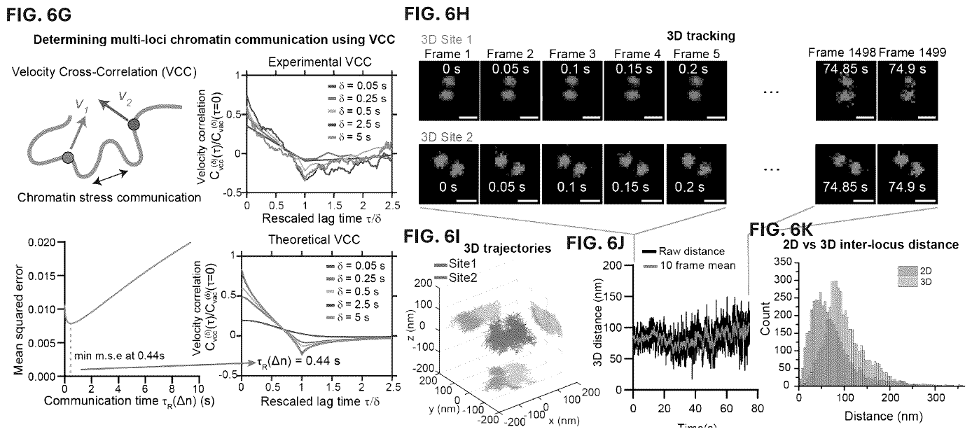

The detailed understanding of the real-time dynamics of pairs of loci in mammalian cells with different genomic distances remains elusive. Because Oligo-LiveFISH offers a versatile platform to label both repetitive and non-repetitive sites with desired distance, we applied multi-color Oligo-LiveFISH to capture chromatin looping dynamics in live cells and to reveal the physical organization of chromatin in 3D. We focused on two sites on Chr3: site 1:195.46 Mb and site 2:195.49 Mb (Table 2, FIG. 6A).

We first quantified the 2D spatial dynamics between site 1 and site 2 (28 kb apart) at a temporal resolution of 50 ms per frame, noting that the two-color imaging with microscope A4 allows both loci to be observed simultaneously, even though they are only ~70 nm apart. Representative trajectories of two sites from two Chr3 alleles in the same cell are shown in FIG. 6B. The movement in the x and y directions reveals a highly correlated motion of the two sites at timescales from 50 ms to several seconds (FIG. 6C) with 2D inter-locus distance fluctuating around a value of ~75 nm over time (FIG. 6D). As a control, tracking beads (FIG. 13F-13G) and nuclear motion (FIG. 13H) at the same temporal resolution precludes the possibility that the correlated motion is due to the stage drift or nucleus movement.

Such detailed time-dependent data now allow a deeper analysis. To test whether fRNP binding affects intrinsic chromatin dynamics, we performed MSD and velocity autocorrelation (VAC) analysis. Both loci exhibited sub-diffusive motion (FIG. 6E), with the MSD fitting revealing effective diffusion coefficients for each: D*=(1.4±0.1)×10⁻³ μm²/s^{α}, α=0.48±0.05, localization error σ=37±1 nm for site 1 and D*=(11.0±0.2)×10⁻⁴ μm²/s^{α}, α=0.43±0.01, localization error o=18±1 nm for site 2. These measurements aligned with previous observations.

The time-dependent position measurements allow for the calculation of velocity, and statistical methods can extract more information based on the velocity correlations at one time compared to another time by the time lag t, expressed by the VAC. VAC analysis (Eq.7-8, see Methods) for both loci revealed characteristic negative peaks at τ=δ for all δ, indicating negative correlations in DNA motion due to both polymer elasticity and medium viscoelasticity, confirming sub-diffusive behavior of chromatin at this time scale (FIG. 6F). In a simple picture, when a thermal kick moves a locus, the stretchiness of the DNA causes a restoring response in the opposite direction. Here δ is the measurement time between locus position measurements used to estimate velocity and τ is the time lag over which the correlation of velocities is examined (see Methods). Moreover, rescaling lag time τ by δ results in a universal curve for all values of δ, which quantitatively agrees with predictions from the analytical theory of fractional Brownian motion (Eq.9) with α=0.38.

The VAC results, combined with the fact that MSD follows a power-law without reaching a plateau, precludes other possible subdiffusive models and unambiguously concludes that the imaged loci move subdiffusively by an fBM model in a viscoelastic nuclear environment (data not shown). This observed fBM dynamics using Oligo-LiveFISH across multiple endogenous genomic sites aligns with those observed using the LacI/LacO approach in yeast chromosomes or the ParB/parS system in bacterial chromosomes, revealing that labeling endogenous chromatin sites using dCas9 fRNP does not affect intrinsic chromatin dynamics. These findings suggest that fBM is likely a universal mode of dynamic genomic DNA movement under cellular conditions. Importantly, fBM helps maintain a given chromatin conformation for extended periods. Furthermore, when two genomic loci are in proximity, fBM facilitates more frequent and recurrent interactions, leading to stronger regulatory responses such as enhancer-promoter interactions. For example, during B lymphocyte development, the immunoglobulin heavy-chain variable (VH), diversity (DH) and joining (JH) segments display fBM, which may provide a physiological advantage by preventing displacement of the DHJH element away from the VH region.

Chromatin loci do not move independently, as biological processes such as chromatin segregation, recombination, and transcriptional regulation involve dynamic communication between chromatin loci for orchestrated action. The motion of different loci on a single chromosome is primarily coupled via stress communication through the elasticity of the chromatin, but stress communication is not instantaneous and depends on the genomic distance between the loci. By performing velocity cross correlation analysis (VCC, Eq.10-11), we characterize the dynamical coupling of the two sites by calculating their VCC at different values of rescaled time lag t/8 (FIG. 6G). By fitting our experimental VCC data to a theoretical framework which models chromatin as a Rouse polymer in a viscoelastic environment, we extracted the relaxation time TR (An)=28 kb) (Eq.14-15), which provides the timescale for stress communication between site 1 and site 2 (28 kb) through the intervening segment, to be 0.44 s (FIG. 6G). By combining robust Oligo-LiveFISH labeling of endogenous chromatin loci, fast tracking, and biophysical modeling, we found that two sites 28 kb apart communicate their dynamic movement in 0.44 s. This time serves as a reference for chromatin coupling over different genomic lengths in the next section.

We further applied the two-color 3D DHPSF microscope C to track the same two sites to confirm the 2D tracking results with the same time resolution of 50 ms (exposure time). The images (FIG. 6H) and trajectories (FIG. 6I, 13I) of a representative loci pair indicates robust tracking of the two sites using Oligo-LiveFISH in 3D for ~1500 frames (~75 s), a rich data set on a time scale that would be missed with much slower time-lapse measurements. The motion in x, y, z directions indicate a correlated behavior of the two loci in 3D space (FIG. 13J), validated by a control measurement using fixed bead tracking (FIG. 13K-13L). A closer look indicates a 3D inter-locus distance of ~80 nm (FIG. 6J).

We compared several biophysical parameters in 2D and 3D. Statistical analysis of the distance distributions indicated a mean separation of 75.1±57.5 nm per localization for the 2D projections (FIG. 6K), with individual trajectory analyses further supporting these findings (mean distance of 72.3±32.0 nm per trajectory, FIG. 13M). When accounting for z positions, the true 3D inter-locus distance between sites 1&2 is larger, as expected, at 101.3±44.1 nm (FIG. 6K). Similar VAC curves are observed in 3D as in the 2D cases (FIG. 13N-13P), confirming the subdiffusive behavior of chromatin with either 2D or 3D methods, while fixed bead motion controls show no correlation (FIG. 13O). Even though the 2D measurements underestimate the actual distance, the intrinsic subdiffusive dynamics and the velocity correlation remain consistent for 2D and 3D measurements.

### Multi-Color Oligo-LiveFISH Tracking of Multiple Sites at Different Timescales Distinguishes Two Modes of Chromatin Interaction

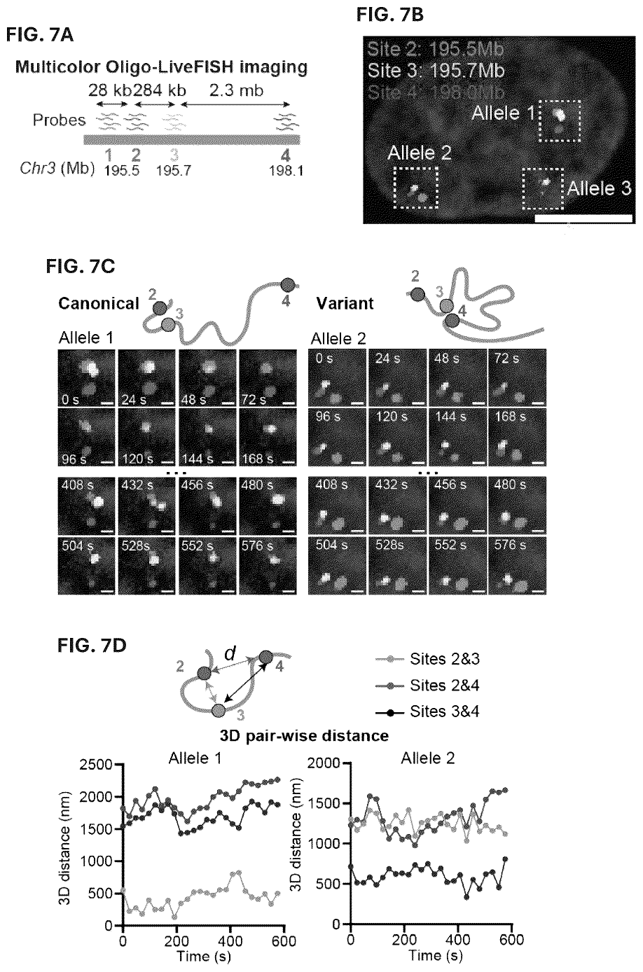

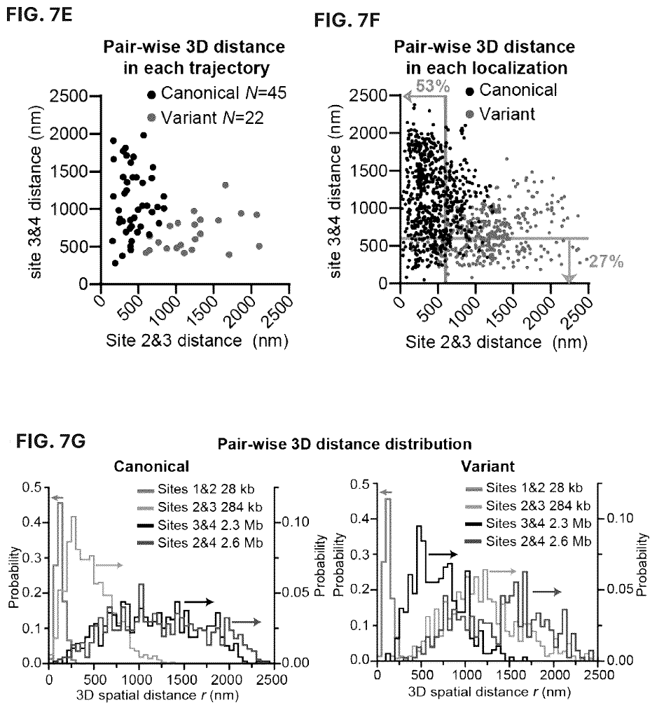

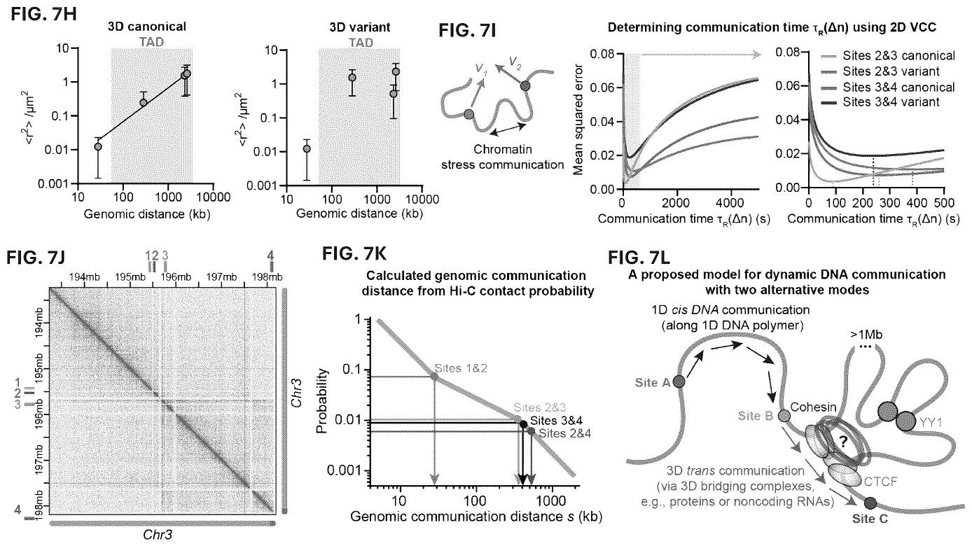

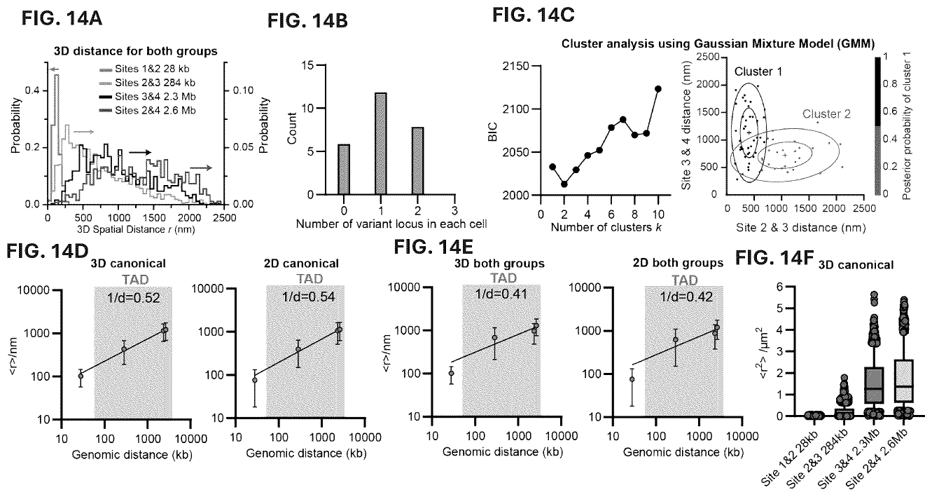

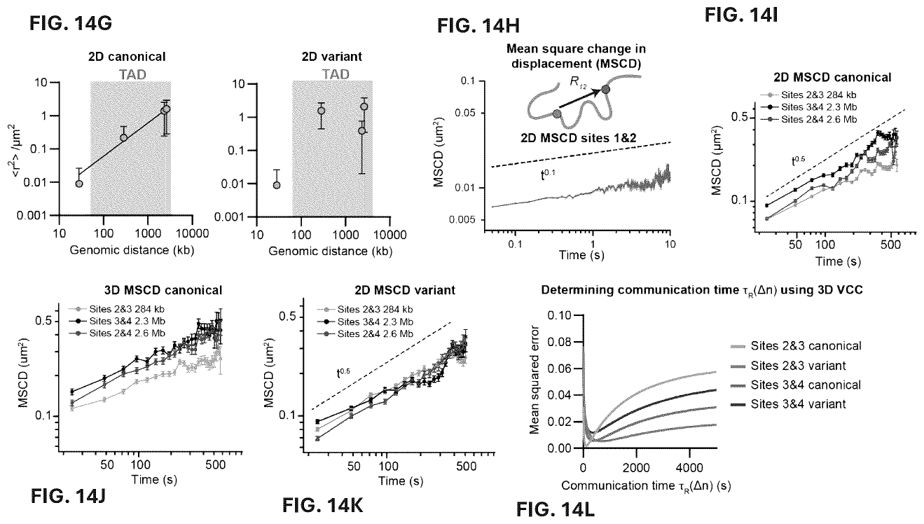

We next expanded to simultaneously image three sites (site 2,3,4, FIG. 7A) using confocal microscope B (data not shown) with A488-, A565- and A647-labeled fRNPs respectively, with 24 s between time points (time-lapse mode). Additional fascinating dynamics of chromatin regions far apart (>200 kb) can be explored at this longer time scale. Sites 2 and 3 are 284 kb apart (proximal pair) and sites 3 and 4 are 2.3 Mb apart (distal pair). A representative image of three locus groups from the 3 alleles in a single U2OS cell is shown in FIG. 7B-C. We expected loci with smaller genomic distances to have smaller spatial distance. On average, our data follow this trend (FIG. 14A), with mean spatial 3D distances between sites 2 & 3, sites 3 & 4, and sites 2 & 4 being 682±471 nm, 963±482 nm, and 1294±542 nm (mean±1SD) respectively.

Surprisingly, we found that a fraction of loci groups demonstrated the opposite behavior, with the distal pair 3 & 4 in close spatial proximity (FIG. 7B-C). In the example cell shown, in allele 1, the proximal pairs 2 & 3 were spatially closer, while in allele 2 the distal pairs 3 & 4 were spatially closer (FIG. 7C). The difference can also be visualized by the 3D pairwise distance in FIG. 7D. To systematically investigate this phenomenon, we assigned the loci group to a “canonical” group if the mean distance of the proximal pair (sites 2 & 3) is shorter than the distal pair (sites 3 & 4) (FIG. 7E). The rest of the trajectories, whose distal pairs are closer in space, are assigned to a “variant” group (FIG. 7E). It appears that this simple procedure breaks all the cases into two groups, but a more careful statistical clustering analysis is described below. Among 67 trajectory groups we counted, ⅓ belonged to the variant group, indicating that the variant behavior is a significant phenomenon related to the intrinsic chromatin organization (FIG. 7E). This behavior is easily observed here with single-cell imaging and would be obscured by bulk sequencing methods.

We counted the number of variant loci groups in cells with exactly three Chr3 loci and obtained similar fractions (FIG. 14B). 23% of cells have all three loci belonging to the canonical group, and 77% have 1 or 2 variant groups. Interestingly, all the cells have at least one Chr3 in the canonical group (FIG. 14B). To quantify the spatial relationship on the same chromosome, we calculated the distance between loci within the same locus group (FIG. 7E-F). A Gaussian mixture model (GMM) was applied to data points and number of clusters, k, was selected as 2 to minimize the Bayesian information criterion (BIC) (FIG. 14C). The resulting bimodal distribution identified by GMM-BIC aligns with the canonical and variant groups we identified, supporting the existence of two distinct chromatin organization groups. (FIG. 7E-F, 14C). Using 600 nm as a cut off distance of spatial proximity, 53% of proximal pair 2 & 3 and 27% of distal pair 3 & 4 were in spatial proximity (FIG. 7F). Taken together, the spatial proximity of sites 2 & 3 (canonical group) and spatial proximity of sites 3 & 4 (variant group), occurs at an approximate probability of 2/3:1/3 respectively.

Next, we quantified the chromatin organization at the single cell level in the two groups separately. In the canonical group, the larger genomic distance exhibited larger mean spatial pair-wise inter-locus distance. The distance of sites 2 & 3 is smaller in canonical groups while the distance of sites 3 & 4 is smaller in variant groups (FIG. 7G). An important question from polymer physics is how the mean inter-locus spatial distance <r> varies with the linear genomic distance s. From theory, <e>~s^{1/d}, where d is the fractal dimension. The scaling exponent 1/d is ½ for ideal chain and ⅓ for ideal fractal-globule polymer model. We have obtained a scaling exponent 1/d of 0.52 for the canonical group (FIG. 14D-14E). This value is larger than the previous reported values in Drosophila and human diploid IMR90 cells but closer to a value predicted by a traditional Rouse model. The Rouse polymer model predicts that mean squared inter-locus distance <r²> scales linearly with the genomic distance with <r²=nb² in which b is the Kuhn length, which defines the statistical average random-walk step size of the polymer segments. By fitting measured <r²> with genomic distance and assuming DNA length of 0.34 nm/bp, we obtain Kuhn length=(1.97±0.02) nm (mean±1S.D.) in the canonical group, indicating that DNA in live mammalian genomes exhibits more flexibility than a free polymer (FIG. 7H, 14F-14G). In contrast, the scaling law fails in the variant group where the distal pair shows a smaller mean square inter-locus distance (FIG. 7H). Similar scaling exponents and Kuhn lengths were obtained when using the 2D spatial distance (FIG. 14D-14G). Further differences characterized by the mean square change in displacement (MSCD) are provided in (FIG. 14H-14K).

Finally, we analyzed chromatin communication over time. By calculating the VCC and comparing it with the modeling results, we obtained the communication time between sites 2 & 3 in the canonical group TR (An-284 kb)=89 s (FIG. 7I, FIG. 14L). Importantly, this value is very similar to a predicted value of TR (An-284 kb)=76 s, assuming that Rouse intersegment relaxation times follows Eq.16 using our previously determined data on the short 28 kb genomic τ_{R} (Δn=28 kb)=0.44 s (See Methods). Additionally, based on the communication time between any two chromatin sites determined via VCC, we can use Eq. 16 to calculate the genomic communication distance, the shortest length between two sites to communicate stress, either through the chromatin segment or through the linking protein complex. The communication distance between sites 2 & 3 in the canonical group is 305 kb (See Methods), very close to the actual genomic distance (284 kb). In contrast, sites 2 & 3 in the variant group have a communication time of 384 s, larger than the predicted value and consistent with their relatively large spatial distance (data not shown).

Surprisingly, while polymer theory predicts that the communication time of sites 3 & 4, whose genomic distance is 2.3 Mb, would be ~2.3 h if the stress is merely communicated through the chromatin segment without looping, our observed communication time in both groups are <300 s, much shorter than the prediction. This corresponds to a genomic communication distance of ~500 kb, significantly shorter than their actual genomic distance (data not shown). This short genomic communication distance suggests that DNA communication across sites 3 & 4 occurs more efficiently via an alternative mechanism, which may be a result of bridging protein and noncoding RNA complexes that create loops to reduce the communication distance.

Oligo-LiveFISH is Consistent with Bulk Hi-C, Revealing Different DNA Interaction on the Single Cell

To better understand our dynamic tracking results from Oligo-LiveFISH, we analyzed corresponding bulk Hi-C data for these sites. The spatial proximity of sites 3 & 4 in the variant group, along with their fast communication time, are consistent with the architectural stripe near site 3 observed in bulk Hi-C data (FIG. 7J). Oligo-LiveFISH, however, uniquely captures dynamic chromatin interactions at the single cell level, revealing heterogeneity among cells and identifying distinct populations with chromatin dynamics that bulk Hi-C or multiplexed DNA FISH cannot resolve.

We speculated that the variant group mainly contributes to the architectural stripe in Hi-C. To confirm this, we calculated pair-wise spatial contact probabilities for the four sites using Hi-C data and converted each contact probability into genomic communication distances based on previously reported simulations (FIG. 7J-K, see Methods). The genomic communication distances of sites 2 & 3 and sites 3 & 4, calculated from sequencing data, are 357 kb and 406 kb, respectively, which closely match the effective genomic lengths we derived from Oligo-LiveFISH dynamic tracking (data not shown).

Taken together, we propose two modes of DNA interaction in 3D space (FIG. 7L): 1D cis-communication and 3D trans-communication. In 1D cis mode, genomic regions primarily communicate via the elastic behavior of the polymer segment. This mode dominates chromatin dynamics in the canonical group up to several hundred kb and can be accurately predicted by the Rouse polymer model. However, 1-D cis-communication along is too slow for distal pairs to communicate over megabase distances during dynamic biological processes. For example, our calculations show that it would take more than 2 hours for two sites 2.3 Mb apart to communicate via 1D cis-communication and this communication time increases rapidly with genomic distance with a power of ~2 (the exact value is 2/α′=2.22, α′=2α=0.9 in our case). Conceptually, this mode of DNA communication would be too slow for many biological processes like DNA damage repair. In contrast, 3D trans mode allows two sites to circumvent the long chromatin polymer and accelerates communication through possible bridging complexes, which we speculate could involve protein and/or RNA. Thus, cells modulate the chromatin interaction dynamics via orchestrated 1D cis- and 3D trans-communication via both DNA polymer and bridging complexes, a new observation made through the unique power of Oligo-LiveFISH combined with dynamic modeling.

### Discussion

In summary, we developed a versatile, high resolution, multi-color chromatin imaging platform, Oligo-LiveFISH, that can flexibly image non-repetitive chromatin loci in diverse living cell types, including primary cells. Because most promoters, enhancers, insulators, introns, and UTRs are non-repetitive regions, successful implementation of our technique will markedly expand the scope of live cell chromatin imaging. By combining purified, nuclease-deactivated dCas9 protein and dye-gRNA probe pools, Oligo-LiveFISH offers a reagent-based approach, bypassing laborious genetic engineering, to target various non-repetitive genomic regions with high resolution in live cells. Our imaging platform integrates computational library design, library-based in vitro RNA transcription, chemical fluorophore labeling and purification. The mammalian genome contains a vast number of potential off-targeting sites when using a pool of gRNAs, particularly considering the mismatch of gRNAs. Therefore, we applied a strict computational design pipeline to filter out sgRNAs that possibly generate a detectable off-target region. Moreover, when combined with SL microscopy and dynamic modeling, Oligo-LiveFISH offers an exciting opportunity to advance our understanding of the principles governing the genome's dynamic structure and function relationship across short- and long-time scales.

A powerful imaging platform must preserve chromatin integrity. Harsh treatments, such as the high temperatures used in FISH or the insertion of large exogenous arrays like LacO/LacI, can disrupt the native 3D genomic organization. In contrast, CRISPR-based systems generally provide a way to image specific chromatin regions without harsh interference to the local chromatin structure. However, due to lower signals in the presences of backgrounds, it has remained a challenge to tag non-repetitive genomic sequences in live cells using previous LiveFISH or other CRISPR-based imaging approaches, restricting their applications. Although several efforts have been made to tag non-repetitive genomic loci based on CRISPR, these systems usually require constructing special cell lines and fine-tuning expression of multiple constructs. Oligo-LiveFISH addresses this major need by offering a simple-to-use, robust, and efficient method to tag essentially any non-repetitive genomic loci in diverse types of cells, including primary cells.

CAS-LiveFISH achieves non-repetitive loci imaging using fluorescent dCas9 and non-fluorescent IVT gRNAs. Compared to CAS-LiveFISH, Oligo-LiveFISH uses organic dyes, which are generally brighter, smaller, and more photostable (typically emitting ~10⁶ photons before photobleaching) compared to fluorescent proteins (~10⁵ photons before photobleaching). Moreover, fluorescent gRNAs typically achieve higher SNR due to the rapid degradation of background gRNAs (FIG. 9D-9F). Additionally, dCas9-EGFP tends to accumulate in nucleoli when concentrations are too high (FIG. 9D-9F), further highlighting the advantages of dye-labeled fRNPs over fluorescent proteins.

Nevertheless, using pools of individually synthesized and purified fluorescent crRNAs is not cost-effective. Oligo-LiveFISH addresses this issue by employing a common fluorescently labeled tracrRNA with an array of IVT crRNAs and delivering a pool of pre-assembled fRNPs in vitro. We also explored improved RNA chemistry to label and purify the IVT crRNA or sgRNA with dyes. A multiplexed IVT crRNA or gRNA library generated from a DNA library significantly reduces the cost and time needed to produce distinctive crRNAs, thus allowing studying many genomic loci in different types of cells in parallel. Successful labeling of crRNA and tracrRNA at both 5′ and 3′ ends also provides potential ability to further increase SNR by using a Fluorescence Resonance Energy Transfer (FRET) or molecular beacon dye pair. Multi-color CRISPR DNA imaging is usually achieved with orthogonal dCas9 proteins or fusing various RNA-binding motifs to the gRNA. In contrast, Oligo-LiveFISH achieves multi-color imaging by simply using tracrRNAs or sgRNAs labeled with different dyes.

Localization uncertainty is inevitable in imaging and can be more pronounced for live cell DNA imaging due to motion blur. Simulation results indicate that an enhancer-promoter contact distances smaller than 50 nm could lead to apparent enhancer-promoter distance of ~150-300 nm due to measurement errors. If not carefully examined, localization error can lead to incorrect mechanisms for enhancer-promoter dynamics. Typical DNA FISH probe sizes are ~50-200 kb, which generally broadens the fluorescence spot and increases localization uncertainties. Uniquely, Oligo-LiveFISH offers flexibility in probe size. The 24 probes in Oligo-FISH usually target a ~3 kb genomic region and 96 probes target ~12 kb regions, making them approximable as a diffraction-limited point. Similar to single-molecule localization microscopy approaches such as PALM and STORM, SL-Oligo-LiveFISH extracts the most probable position of such probes with ~10-30 nm localization precision (FIG. 5D). If the dynamics of larger chromatin regions are of interest, Oligo-LiveFISH could simply be adapted to paint this region by designing a larger gRNA pool.

To develop effective principles for designing “efficient” probes, we applied machine learning to identify key parameters influencing imaging efficiency. We found that probe number and chromatin accessibility are the most critical parameters. When designing gRNA pools to tile a region, the pool unavoidably contains both highly “efficient” and less “efficient” gRNA species. Using a constant total concentration for delivery, the less efficient gRNAs can dilute the overall effectiveness of the pool. Therefore, to enhance the binding efficiency of the gRNA pool to the target region, we summarize a set of design principles that can serve as a general guide for designing gRNA pools (see Methods) to benefit users in CRISPR-based imaging and perturbation.

DNA communication is significant for many biological processes. Determining the position of chromosome loci and inter-locus distances is critical for understanding chromatin topology and its roles in gene regulation. Incorporating the dimension of time into chromatin research can significantly enhance our understanding. Our study of the dynamic position of several sites on the same chromosome, with varying genomic distances, revealed that some distal pairs are unexpectedly close in space and communicate faster than predicted. By combining our imaging data and dynamic modeling, we proposed two modes of chromatin communication in live cells. While polymer theory predicts the dynamics across genomic sites up to ~300 kb, where DNA primarily communicates through elastic polymers via 1D cis-communication, our experimental results and computational model explain the observed accelerated communication beyond Mb scales via 3D trans-communication. Various structural proteins in the genome can form loops, bridging structures, or compartments, which can aid 3D trans-communication. For example, the boundaries of topologically associating domains (TADs) are usually delimited by CCCTC-binding-factor (CTCF) which stall cohesin. Previous study using super-resolution microscopy demonstrated that CTCF forms clusters that often couple with cohesin to create complexes, potentially playing a role in the observed 3D trans-communication and facilitating exchange of dynamic information between sites in real-time. Future studies should investigate the generality and structural origins of these phenomena, particularly how chromatin is organized in 3D to achieve such rapid communication between distal pairs. Among options of microscopy, we suggest using true 3D imaging over 2D projections for these questions.

With new methods to capture chromatin looping dynamics, it becomes possible to address fundamental biological questions, such as the functional relevance and causality between dynamic enhancer-promoter communication and real-time transcription. Oligo-LiveFISH could enable multimodal imaging when combined with RNA and protein imaging, and it has the potential to reveal the causality between dynamic genome structure and transcription when integrated with perturbation techniques like inducible CRISPR interference/activation (CRISPRi/a).

### Methods

### Cell Culture

The U2OS and HeLa cells were cultured in DMEM with GlutaMax (Thermo Fisher Scientific, 10569-044) supplemented with 10% FBS (Sigma-Aldrich, F0926). T cells were cultured in 0.22 μm sterile-filtered RPMI with L-Glutamine and 25 mM HEPES, supplemented with 10% FBS, 100 U mL⁻¹ penicillin, and 100 μg mL-1 streptomycin (Gibco). Human recombinant IL-2 (STEMCELL Technologies) was provided at 100 U mL-1. Dynabeads were magnetically removed on day 3 of culture. A human embryonic stem cells (hESC) parent line, H1 (WiCell, #WA01), that harbors the stable and random integration TetO-NGN2-Puro was used. NGN2-H1 cells were cultured according to standard stem cell culturing protocols. In brief, cells were grown in mTeSR Plus medium with the accompanying supplement (STEMCELL Technologies, #100-0276) and the media was changed every 2 days. Cells were passaged when colonies reached the appropriate sizes and the edges of each colony almost touched the other. When passaging the NGN2-H1 cells, a 6-well plate was coated with iMatrix-511 (amsbio, #AMS.892012) and incubated for 1 h at 37 °C in a CO2-regulated tissue culture incubator. Cells were detached using ReLeSR (STEMCELL Technologies, #100-0484) according to the vendor's protocol and ReLeSR activity was quenched using mTeSR Plus medium and cells. The iMatrix-511 solution was removed from each respective well of a 6-well plate and approximately 80,000-100,000 cells were seeded into each well. The passaged NGN2-H1 cells were grown in mTeSR Plus that was supplemented with 10 μM of rock inhibitor, Y-27632 (dihydrochloride) (STEMCELL Technologies, #72302) for 12-24 hours, followed by a media change with fresh mTeSR Plus (without rock inhibitor). All cells were cultured at 37 °C and 5% CO₂ in a humidified incubator.

### Primary Human T Cell Isolation and Expansion

Primary human T cells were isolated as previously described⁷⁴. Briefly, buffy coats derived from anonymous healthy blood donors were purchased from the Stanford Blood Center. Primary human T cells were isolated using the EasySep Human T cell Isolation kit (STEMCELL Technologies, #17951) according to the manufacturer's protocol with Ficoll-Paque PLUS (GE Healthcare, #17144002) and SepMate-50 tubes (STEMCELL Technologies, #85450). Isolated T cells were immediately cryopreserved at 2-4×10⁶ cells per vial in FBS supplemented with 10% DMSO (Sigma). Cryopreserved T cells were thawed and activated on the same-day using Human T-Activator CD3/CD28 Dynabeads (Gibco, #11132D) at a 1:1 bead-to-cell ratio.

Design Tiling crRNAs/sgRNAs Targeting Non-Repetitive Regions.

To design dense crRNA/sgRNA targeting non-repetitive regions for imaging, we retrieved DNA sequences from human genome hg38 at multiple loci, including regions on Chr3, Chr8, Chr13, Chr14, and ChrX (region coordinates in Table 2). The enhancer regulating MYC and FOS were predicted enhancers by the active-by-contact (ABC) model. Home script was used to design all the possible crRNAs/sgRNAs with different spacer lengths, from 13 bp to 20 bp. First, all these crRNAs/sgRNAs had the NGG PAM sequences and started with G for higher in vitro transcription efficiency. Additionally, the GC content was between 35% and 80%. Finally, these crRNAs/sgRNAs were filtered if they contained sequences: TTTT; 10×G; CTCGAG; GCTNAGC; CCANNNNNNTGG. To ensure no imaging signal caused by off-target crRNAs/sgRNAs, we set up a pipeline to remove crRNAs/sgRNAs which possibly target other regions with a short interval distance. Cas-OFFinder was used to search all potential off-target sites in human genome hg38 with ≤1 mismatch in spacer length <16 bp, with ≤2 mismatches in spacer length ≥16 bp. We observed that some short crRNAs/sgRNAs had many off-target loci. Thus we removed these crRNAs/sgRNAs with >100 off-target loci. Next, we defined off-target regions, which contained ≥2 off-target loci with interval distance <2000 bp. All the crRNAs/sgRNAs involved in these off-target regions were filtered. Finally, to control the interval distance between tilling crRNAs/sgRNAs, we removed crRNAs/sgRNAs which were too close to the previous crRNAs/sgRNA (<10 bp).

Calculate/Retrieve Epigenomic Occupancy in sgRNA Targeting Regions.

To examine the occupancy of histone modification (HM) profiles and chromatin accessibility at probe targeting site, the genome-wide profiles of HMs in U2OS and HeLa cell line were generated from Gene Expression Omnibus (GEO) database. The sample ID in GEO database for each HM and ATAC-seq in U2OS cells are listed in the table below. Data downloaded from GEO were processed with Bowtie (60) and MACS2 (61) to generate the sequence depth normalized bigwig file. Then, we retrieved the enrichment signal of each HM at each probe targeting site.

| Sample | GEO ID |
|---|---|
| H3K27ac | GSM3734223 |
| H3K4me1 | GSM4861712 |
| H3K18ac | GSM1709274 |
| H3K56ac | GSM1709278 |
| H3K9ac | GSM1709280 |
| H3K9me3 | GSM1709282 |
| H3K4me3 | GSM4079841 |
| ATAC-seq | GSM4133297 |

### Preparation of DNA Oligo Pools

The DNA library was either purchased from IDT or Twist Bioscience. The DNA libraries from Twist Bioscience are in the form of oligo pools. Each DNA oligo contains a unique forward adaptor sequence for PCR, a T7 promoter, a spacer sequence, and a common crRNA/sgRNA backbone (Table 1). The library was amplified by PCR (Q5 Hot Start High-Fidelity 2× Master Mix, NEB, M0494S) using a unique forward primer and a general reverse primer. The DNA libraries from IDT are in the form of ssDNA plates and we mix a subset of them each time to form the sub libraries. Each DNA oligo contains a T7 promoter, a spacer sequence, and a common crRNA/sgRNA backbone. The library was amplified by PCR using the library as the forward primer and a general reverse primer. The PCR product was purified by agarose gel electrophoresis (Monarch DNA Gel Extraction Kit, NEB, T1020S) and the purified PCR products were used as templates for in vitro transcription.

### RNAs Synthesis, Purification, and Preparation

The universal 5′-dye-tracrRNAs and unlabeled tracrRNA were synthesized by IDT. The unlabeled crRNA, and sgRNA were synthesized in vitro by T7 RNA polymerase (HiScribe™ T7 Quick High Yield RNA Synthesis Kit, NEB, E2050S). The IVT RNA was treated with RNase-free DNaseI to digest the DNA template for 20 min and purified using RNA Clean & Concentrator™-25 (Zymo Research, R1018). The concentration of IVT RNA was measured based on absorption (Nanodrop). crRNA and tracrRNA were annealed in RNA folding buffer (20 mM HEPES, pH 7.5 and 150 mM KCl) with a molar ratio 2:1-5:1 with the component without dye in access, or with an equal molar ratio if both tracrRNA and crRNA are labeled with dye. The crRNA: tracrRNA complex was incubated at 95 °C for 5 min, 70 °C for 10 min, gradually cooled down to room temperature and supplemented with 1 mM MgCl2, then kept at 40 °C for 5 min and gradually cooled down. The sgRNA was incubated at 70 °C for 10 min, gradually cooled down to room temperature and supplemented with 1 mM MgCl2, then kept at 40 °C for 5 min and gradually cooled down. We included an A-U flip (Table 1) in the original Cas9 crRNA: tracrRNA backbone sequence based on previously reported improvement with the sgRNAs for imaging applications.

### Dye Labeling of RNAs

To label the 3′ end of IVT RNA with fluorescent dye, yeast poly(A) polymerase and click chemistry were applied. Briefly, 1-10 μM RNA purified from IVT was mixed with 2 mM 2′-Azido-2′-dATP (NU-976S, Jena Bioscience), 1200 U yeast ploly(A) polymerase (PAP, RNT-006-S, Jena Bioscience), and 1×PAP reaction buffer at 37 °C overnight in an RNase free environment followed by purification using RNA Clean & Concentrator™-25 (Zymo Research, R1018). The click reaction was then performed with the purified 3′-azido-modified RNA, 1 mM DBCO-dye (Click Chemistry Tools), and 1 U/uL RNase inhibitor (M0314S, NEB) at 37 °C for 1 hour, followed by purification using RNA Clean & Concentrator™-25 (Zymo Research, R1018).

To label the 5′ end of IVT RNA with fluorescent dye, 120 mM GMP solution, instead of H2O was added to the IVT system. 13.5 μL purified IVT RNA was dissolved in 1.5 μL 10×PBS buffer (1×PBS final concentration). To label the RNA with-Cy3, 0.6 μL 0.8 M Cy3 amine (BP-22558, BroadPharm) was added to 9.4 μL 0.1 M imidazole (pH=6) to form Cy3 amine/imidazole solution. 1.44 mg (1-ethyl-3-(3-dimethylamino) propyl carbodiimide, hydrochloride) (EDC) was weighed into 1.5 mL tube, followed by adding 15 μL RNA solution (diluted in 1×PBS) and immediately adding 10 μL Cy3 amine/imidazole solution. The solution was vortexed and spun down. 40 μL 0.1 M imidazole (pH=6) was further added to the system, followed by briefly vortexing and incubating at 37 °C overnight. The system was purified by RNA Clean & Concentrator™-25 (Zymo Research, R1018) for three times. To label the RNA with Alex Fluor-647 (AF-647), 2 μL ethylenediamine was added to 98 μL 0.1 M imidazole (pH=6) to form ethylenediamine/imidazole solution. 1.44 mg (1-ethyl-3-(3-dimethylamino) propyl carbodiimide, hydrochloride) (EDC) was weighed into 1.5 mL tube, followed by adding 15 μL RNA solution (diluted in 1×PBS) and immediately adding 10 μL ethylenediamine/imidazole solution. The solution was vortexed and spun down. 40 μL 0.1 M imidazole (pH=6) was further added to the system, followed by briefly vortexing and incubating at 37 °C overnight. The system was purified by RNA Clean & Concentrator™-25 (Zymo Research, R1018) once and elute in 60 μL H2O. The RNA solution was further added 6 μL 3M CH3COONa (pH=5.5) and 200 μL EtOH prechilled at −80 °C and stored at −80 °C overnight. The solution was centrifuge in 4 °C for 20 min with 13000 rpm to form the RNA precipitate. The RNA precipitate was washed with 70% EtOH (−80 °C) for three times, air dried and dissolved in H2O. 18 uL NH2-RNA solution was added by 2 μL 1M NaHCO₃(pH=8.5) and 2 μL Alex Fluor-647 NHS ester (Invitrogen, A37573, dissolved in DMSO), and incubated at 37 °C overnight. The system was purified by RNA Clean & Concentrator™-25 (Zymo Research, R1018) for three times.

Assembly and Live Cell Delivery of fRNP Complexes

RNP delivery by electroporation was performed using Neon Transfection System 10 μL kit (Thermo Fisher, MPK1096). For delivery of fluorescently labeled RNP complexes, 0.5-10 μmol of dye-labeled crRNA: tracrRNA or sgRNA were mixed with equal molar amount of dCas9-EGFP (GenScript, T2211048) or x1.2-1.5 molar amount of dCas9 (IDT, 1081067), and then incubated at room temperature for 10 min to allow for fRNP assembly. The assembled fRNP complexes were transfected into 2-4×10⁵ suspended cells using the standard protocol for the Neon Transfection System 10 μL kit. Electroporation was performed in U2OS cells at 1400V/15 ms/4 pulses, in activated T cells at 1400V/10 ms/3 pulses, in hESC iNs at 1300V/20 ms/3 pulses, and in HeLa cells at 1400V/15 ms/4 pulses. For U2OS and HeLa cells, the transfected cells were immediately plated in either 8-well u-plates (ibidi, 80827-90) or 24-well μ-plates (ibidi, 82426) containing prewarmed culture medium. Hoechst 33342 was added to cells at 0.1 μg/mL to stain the nucleus 6 hours after transfection or right before imaging. The growth medium was changed to imaging medium [BrightCell MEMO Photostable Media (Sigma, SCM144) containing 10% FBS] for live-cell imaging.

The Oligo-LiveFISH procedure was optimized for suspension cells. For suspension primary human T cell imaging, transfected T cells were recovered in pre-warmed complete medium for 3 hours. Cells were then stained with 0.1 μg/mL Hoechst 33343, washed once, and then transferred to 8-well μ-plates (ibidi, 80827-90) or 24-well μ-plates (ibidi, 82426) containing prewarmed imaging medium. Plates were pre-treated with 1 μg/ml poly-D-lysine (PDL) (Thermo Fisher Scientific, A3890401) for 1 hour at 37 °C to accelerate cell attachment. Cells were incubated for another 3 hours to facilitate attachment before imaging.

Plasmid Transfection and Sequential Delivery of fRNP Complexes

To further confirm the specificity of Oligo-LiveFISH, we labeled telomere simultaneously using two methods: (1) transfecting a plasmid (pSLQ14825) expressing TRF1-Halo Tag driven by PGK promoter; (2) delivering Oligo-LiveFISH probes with crRNA targeting telomere annealed with 5′-dye-tracrRNAs. Telomeres are composed of tandem repeats bound by a six-protein complex known as shelterin and TRF1 is one component of the shelterin. Plasmid pSLQ14825 were transfected to U2OS cells with Lipofectamine 2000 (Thermo Fisher Scientific, 11668030) according to vendor's protocol. 24 hours later, fRNPs of Oligo-LiveFISH were delivered to the cells by electroporation using Neon Transfection System as above and the transfected cells were immediately plated in 24-well μ-plates (ibidi, 82426) containing prewarmed culture medium. Before imaging, 200 nM Janelia Fluor 503 HaloTag Ligand (Promega, HT1010) was added to cells for 30 minutes and Hoechst 33342 was added to cells at 0.1 μg/mL to stain the nucleus. The growth medium was changed to imaging medium [BrightCell MEMO Photostable Media (Sigma, SCM144) containing 10% FBS] for live-cell imaging.

### Sequential (Oligo-) LiveFISH-FISH Experiment.

The FISH probes (Empire Genomics) were designed to target a neighboring region of the (Oligo-) LiveFISH probes (Table 2). Because the harsh condition in FISH disrupts the signals of (Oligo-) LiveFISH probes, we developed a sequential protocol in which the (Oligo-) LiveFISH signals were recorded first in the microscope and FISH signals were taken again by finding the same cells using a gridded coverslip. Specifically, the (Oligo-) LiveFISH experiment is conducted as above. The cells were cultured at 37 °C in growth medium on gridded glass coverslips Grid-50 (ibidi, 10187) after the delivery of the (Oligo-) LiveFISH probes by electroporation. The coverslip has imprinted grid with a repeat distance of 50 μm to find the same cell. 4-6 h after electroporation, the growth medium was removed, and the cells were washed once by 1×PBS solution. The chamber was incubated with 4% formaldehyde solution for 10 min at room temperature and then washed twice with 1×PBS solution. Hoechst 33342 was added to cells at 0.1 μg/mL to stain the nucleus. The samples were imaged under microscope with both fluorescent channels (for (Oligo-) LiveFISH signals) and bright field channels (for grid coordinates). After imaging, the medium was changed to 70% EtOH at −20 °C overnight. The coverslip was air dried, washed with 1×PBS once, incubated with 0.1 mg/mL RNase A (Thermo Fisher Scientific, EN0531) in 1×PBS solution for 1 hour at 37 °C, washed with 1×PBS, 70% EtOH, 100% EtOH sequentially and then air dried. 2 μL FISH probe and 13 μL hybridization buffer (Empire Genomics) were mixed and added to the cellular area on the coverslip, which was further assembled with a 24 mm×60 mm coverslip (Globe Scientific, 1419-10). For the three-color FISH experiment, 1.5 μL FISH probe of each chromosome and 10.5 μL hybridization buffer were mixed to make the final 15 μL solution. The assembled coverslip was wrapped by aluminum foil and heated for 10 min at 95 °C and then incubated in a humidified chamber at 37 °C overnight. The sample was washed twice with 50% wash buffer (50% formamide and 0.1% TritonX-100 in 2×SSC buffer), incubated in 50% wash buffer for 10-20 min in dark, washed once with 2×SSC at RT, and mounted onto slides with VECTASHIELD PLUS Antifade Mounting Medium (Vector Laboratories, H-1900-10) for imaging. The colors of FISH probes and (Oligo-) LiveFISH probes are different to exclude the possibility that the FISH signal is the remaining signal from (Oligo-) LiveFISH probes.

### Induced Neuron Differentiation and Electroporation

NGN2-H1 hESCs were induced into neurons based on established protocols with slight adaptations. In brief, Accutase (STEMCELL Technologies, #07922) was used to detach NGN2-H1 cells. The singularized cells were seeded onto Matrigel-coated 6-well plates at a density of approximately 400,000 cells per well. “Pre-differentiation” medium was prepared to achieve the final working concentrations: KnockOut DMEM/F12 medium (Gibco, #12660012), 1×MEM NEAA (Gibco, #11140050), 1×N2 supplement (Gibco, #17502048), 1× GlutaMAX (Gibco, #35050061), 1 μg/mL doxycycline, 1 μg/mL Laminin (Sigma-Aldrich, #L02020), 1 μg/mL puromycin. The pre-differentiation medium was supplemented with rock inhibitor on Day 1. The cells underwent medium change with pre-differentiation medium without rock inhibitor on Days 2 and 3. A 24-well glass bottom imaging plate was coated with 0.1 mg/mL of poly-D-Lysine (A3890401) for 24 h at 37 °C, rinsed thoroughly with sterile double distilled water, coat ed with 200 μL of 1 μg/mL of laminin per well, and incubated at 4 °C overnight. On day 4 (also called “DIV0”), the cells were pushed into neuron induction. First, cells were detached with Accutase according to the vendor's protocol. Accutase was next inactivated with complete “maturation” medium: Neurobasal-A (without phenol-red, Gibco, #12349015), 1×B27 supplement (Gibco, #17504044), 1×MEM NEAA, 1× GlutaMAX, 1 g/mL doxycycline, 1 μg/mL laminin, 10 ng/mL BDNF (PeproTech, #45002), 10 ng/mL NT3 (PeproTech, #45003) in a 1:4 ratio of Accutase to medium. The cells were pelleted at 300×g for 2 minutes. The cells were rinsed once with DPBS, counted, centrifuged at 300×g for 2 minutes, and resuspended in electroporation buffer R from the Neon Transfection System 10 μL kit (Thermo Fisher, MPK1096). Meanwhile, 500 μL of maturation medium was added to each well of a 24-well plate. The cells were electroporated at 1.5-2×10⁵ cells per electroporation condition, washed once and seeded directly into their respective wells within the PDL/laminin-coated 24-welled plate. Hoechst 33342 was added to cells at 0.1 μg/mL to stain the nucleus 6 hours after transfection or right before imaging.

### Cell Cycle Analysis Assay

Primary human T cells were thawed and split in two groups, with one group activated with Human T-Activator CD3/CD28 Dynabeads at a 1:1 bead-to-cell ratio. On day 3 of culture, 5×10⁵ T cells and U2OS cells were stained with Vybrant DyeCycle Ruby Stain (Invitrogen) according to the manufacturer's protocol in 1 ml RPMI with L-Glutamine and 25 mM HEPES, supplemented with 10% FBS, 100 U mL-1 penicillin, and 100 μg mL-1 streptoMYCin (Gibco). After staining, cells were immediately analyzed via flow cytometry, which was conducted on a CytoFLEX S (Beckman Coulter). For all samples, a minimum of about 1×10⁴ events was collected within the final gated population-of-interest. Data were analyzed using FlowJo v10 (BD Biosciences).

### Cell Proliferation Assay

U2OS cells and primary human T cells thawed same-day were stained with CellTrace Far Red dye (Invitrogen) according to the manufacturer's protocol. Immediately after staining, freshly thawed primary human T cells were split into two groups, with one group activated with Human T-Activator CD3/CD28 Dynabeads (Gibco) at a 1:1 bead-to-cell ratio. Cells were incubated for 5 days at 37 C and 5% CO2. Dynabeads were magnetically removed before analysis. Cells were washed twice with FACS buffer (DPBS supplemented with 2% FBS and 1 mM EDTA (Gibco)) prior to flow cytometry, which was conducted as previously described.

### Microscopy Imaging

Six different microscopes were used for imaging in this study (data not shown).

Microscope A1: Widefield microscopy was performed with a Leica dMi8 microscope equipped with a Leica-DFC9000GT-VSC10166 camera, a SOLA Light Engine, and a live cell imaging environment control system. Images were taken using LAS X system with Z stacks at 0.3 μm step size. The magnified camera pixel size is 0.103 μm/pixel with the HC PL APO 63× oil objective (NA=1.40). For live cell imaging, the cells were kept at humidified 37 °C and 5% CO2.

Microscope A2: Single-color live-cell 2D tracking of loci and some of loci bleaching data were acquired using a custom-built single-molecule inverted widefield microscope. A 100 mW 561 nm CW laser was used to excite the sample. The laser beam was magnified twice by a pair of lenses before entering the backport of the microscope body (Olympus IX71). A Köhler lens was placed near the backport to ensure widefield illumination after the excitation light was collimated by the objective (Olympus, UPlanSAPo 100×/1.4 oil). Fluorescence was separated from the excitation light using a multi-band dichroic mirror (Semrock, Di01-R405/488/561/635-25×36) and then focused by a tube lens (f=180 mm) to an intermediate image plane. The fluorescence emission was further relayed by a pair of lenses (f=90 mm for both) in a 4f optical configuration, as previously reported. This emission pathway enables 3D imaging experiments using dielectric phase masks, but for these experiments, no phase masks were inserted into the emission pathway, enabling standard 2D imaging. The emission light was further filtered with a combination of a 561-notch filter (Chroma, ZET561NF) and a 605/70 band-pass filter (Chroma, ET605/70M) placed in the 4f emission pathway. The fluorescence light was finally focused by the last 4f lens onto an EMCCD camera (Andor, iXon897) with a pixel size of 160 nm in sample coordinates. To image the nucleus, the samples were excited with a 405 nm laser (Coherent Cube), and the fluorescence was collected and filtered using the same dichroic mirror mentioned earlier, along with a 440 long-pass filter (Chroma, HQ440LP). For live-cell 2D tracking, samples were imaged at 37 °C using a stage-top incubator (INUB-PI-F1, Tokai Hit) and illuminated at approximately 3-10 W/cm². The camera exposure time was set either 18 ms (FIG. 5G-H) or 50 ms (FIG. 13C).

Microscope A3: Loci bleaching data were acquired using a custom-built single-molecule widefield microscope, consisting of an inverted Nikon Diaphot 200 frame with an oil-immersion objective 60×/1.35 NA (Olympus UPLSAPO60XO) and an EMCCD camera (Andor iXon Ultra 897). 642 nm and 560 nm 1 W continuous-wave (CW) lasers (MPB Communications Inc.) were used for excitation of Cy5 and Cy3, accordingly, and a 405 nm 50 mW CW diode laser (Coherent OBIS) was used for Hoechst 33342. All laser beams were expanded, co-aligned in free space and coupled into a square-core multi-mode fiber with a shaker for speckle reduction (Newport F-DS-ASQR200-FC/PC). The output tip of the fiber (200×200 μm2 core size) was imaged with a 10×/0.25 NA objective and magnified to achieve a square sample illumination region of 47.6× 47.6 μm2 with a constant intensity in the image plane of the main objective. The fluorescence was split from the excitation light with a multi-band dichroic mirror (ZT405/488/561/640rpcv2, Chroma). The fluorescence light was then filtered with a combination of the ZET635NF and ET685/70M filters (Chroma) for Cy5, with a combination of the FF01-609/57-25 and 561LP EdgeBasic filters (Semrock) for Cy3, or with the FBH450-40 filter (Thorlabs) for Hoechst 33342. The image of the sample was focused with a tube lens (f=400 mm) on the EMCCD camera, providing a pixel size of 117×117 nm2 in sample coordinates. The sample was mounted on two stacked piezo stages (PI U-780.DOS for coarse and P-545.3C8S for fine movement). To correct axial (Z direction) drifts, an infrared beam (Thorlabs S1FC808) was directed toward the sample through the main objective. This beam was aligned so that its reflection from the coverslip-imaging buffer interface moves laterally when the sample moves axially. During imaging, the Z position of the fine stage was directed to move proportionally to the shift of the reflected beam, compensating for Z drifts.

Microscope A4: Live-cell two-color 2D tracking of loci labeled with AZ488 and Cy3 was performed with the Microscope C with the following modifications. The 647 nm notch filter was replaced with a 488 nm notch filter (Semrock, NF03-488E-25). The emission pathway dichroic was replaced with a 560 nm long-pass (Semrock, FF560-FDi01). Bandpass filters were selected appropriately for each dye (Semrock, FF03-525/50-25; Chroma, ET605/70M). The double-helix phase masks were removed to produce the open aperture point-spread function. Each cell was first imaged with 6 W/cm² intensity of 405 nm with 50 exposure time and 272 EM gain to locate the nucleus. Loci were then imaged simultaneously with 27 W/cm² intensity for 488 nm and 29 W/cm² intensity for 561 nm with 50 ms exposure time and 272 EM gain until the loci photobleached. After imaging cells, 100 nm Tetraspeck beads were imaged to fully sample the microscope field-of-view to compute the transformation function to register localizations in each channel.

Microscope B: Confocal microscopy was performed at the Stanford University Cell Sciences Imaging Facility. Most images were taken with a Nikon TiE inverted spinning disk confocal microscope (SDCM) equipped with a Photometrics Prime 95B sCMOS camera and a CSU-X1 confocal scanner unit with microlenses. The magnified camera pixel size of SDCM is 0.183 μm/pixel with 60×PLAN APO IR water objective (NA=1.27). The Hoechst, gRNA-A488 (or dCas9-GFP and Fluorescein), gRNA-Cy3 (or gRNA-A565) and gRNA-Cy5 (or gRNA-Atto647 and gRNA-AF647) fluorescence were excited using 405 nm, 488 nm, 561 nm, and 642 nm lasers lines and collected with a 445/20, 525/30, 605/15 and 676/29 emission filters (Semrock) respectively. FIGS. 4B, 4K and 8C were taken using the Nikon Ti2 Crest SDC microscope equipped with a Photometrics Kinetix camera, a Perfect Focus (PFS) focus lock system, and 60× Plan Apo oil objective (NA=1.40), and 365 nm, 488 nm, 561 nm, and 640 nm lasers. For both SDC microscopes, the pinhole size is 50 μm. Z-series images were taken using a piezo Z-axis stage (Mad City Labs) and the NIS Elements software with Z stacks at 0.3 μm step size except the three-color tracking in Figure, which used 0.2 μm step size for better axial position. When imaging 4 color channels (FIG. 7), this microscope captures 5 z slices for 30 frames, with each frame completed every 24 s due to filter changes. For other situations, the time between position measurements is changed appropriately.

Microscope C: Live-cell 3D tracking of loci labeled with Cy3 and AlexaFluor647 was performed with a home-built 3D single-molecule imaging microscope. CW 647 nm (MPB Communications), CW 561 nm (Coherent Sapphire), and CW 405 nm (Coherent OBIS) lasers were coaligned and focused onto the fiber input of 200-μm diameter circular-core multimode fiber de-speckler (Newport, F-DS-AFS200-FC/PC). The fiber output was imaged with a 10× objective (Newport, LIO-10) and relayed to the sample plane through the back port of the microscope body (Olympus, IX71) via 3 achromatic lenses, a quad-pass dichroic (Chroma, ZT405/488/561/640rpcv2), and the objective lens (Olympus, UPlanSAPo 100×/1.4 oil), producing a 35-μm diameter flat, circular illumination profile. Fluorescence was collected with the objective and focused with the tube lens before passing through a 561-notch filter (Chroma, ZET561NF), a 647-notch filter (Chroma, ZET647NF) and entering a two-channel 4f system. The first 4f lens (f=90 mm; Qioptiq, G322389000) was positioned one focal length away from the intermediate image plane after the tube lens. After this lens, a 640 nm long-pass dichroic (Semrock, FF640-FDi02-t3-25×36) was used to split emission into two color channels. A double-helix phase mask (2012-2.4 μm-DH2.7-615-SQ, fabricated in-house81; 2018-2.4 μm-DH2.7-686-DHO, Double-Helix Optics) was placed one focal length after the first lens in each arm of the emission pathway, followed by bandpass filters (Chroma, ET605/70M; Chroma, ET700/75M) for each channel. A second 4f lens (f=120 mm; Edmund Optics, 32921) was placed 120 mm after the phase masks in each channel. Finally, emission from each arm of the emission pathway was reflected off the two faces of a knife-edge mirror to form an image of each channel onto separate portions of an EMCCD camera (Andor, iXon 897 Ultra) with a pixel size of 124 nm in the 605/70 channel and 117 nm in the 700/75 channel. Each cell was first imaged with 6 W/cm² intensity of 405 nm with 50 ms exposure time and 272 EM gain to locate the nucleus. Loci were then imaged simultaneously at 29 W/cm² for 561 nm and 37 W/cm² intensity for 647 nm with 50 ms exposure time and 272 EM gain until the loci photobleached. After imaging cells, 100 nm Tetraspeck beads (Invitrogen, T7279) were imaged to fully sample the microscope field-of-view to compute the transformation function to register localizations in each channel (see Calibration Bead Sample Preparation for details). A calibration z-stack of 100 nm Tetraspeck beads was also acquired simultaneously in each color channel (3.2 μm range, 50 nm steps).

To assess the impact of microscope stability and sample drift on observed loci motion, 100 nm Tetraspeck beads were imaged with 561 nm and 647 nm excitation in two color channels with Microscopes A4 (2D) and C (3D), allowing us to see the same bead in both color channels at the same time. Excitation power and white light background were selected to produce similar signal and background levels as the chromosomal loci. Beads were imaged with the same camera parameters as the chromosomal loci. Beads were then localized and registered onto each other.

To assess the drift of typical nuclei of live U20S cells, cells were stained with 0.1 μg/mL Hoechst and then mounted on Microscope A4. Nuclei were imaged with ~6 W/cm² 405 nm. Nucleus images were then thresholded and converted to binary to isolate the nucleus. The center of mass of the binary nucleus images was then calculated and plotted against time as a measure of live-cell nuclear drift.

Calibration Bead Sample Preparation: CellVis sample chambers (D29-20-1.5H) were incubated with 1M potassium hydroxide for 10 minutes, washed with water, incubated with 0.44% polyetheylenimine for 10 minutes, washed with water, and dried with nitrogen. 100 nm Tetraspeck beads (Invitrogen, T7279) were diluted in hot agarose (1% w/v) and placed onto the prepared sample chamber. The agarose-bead mixture was then allowed to cool and solidify before the chamber was mounted on the coverslip for imaging.

### Image Processing and Quantitative Analysis.

Image processing was performed in Fiji (ImageJ) and Matlab. A single microscope plane showing fluorescence of labeled genomic loci or the projection of adjacent Z planes showing maximum loci fluorescence is shown in figures. The contrast levels were kept the same for each channel and merged. Line scans were performed using the “Analyze/Plot Profile” function in Fiji. To compare the fluorescence intensity in different channels with different backgrounds, the relative fluorescence intensity was calculated by setting the background fluorescent intensity as 1. For FIGS. 1G, 3B, 3I, 3L, 3M, 4D, 4K, 8C, 12H and 13B, the Oligo-LiveFISH fluorescent signal only within the nucleus is shown. To quantify the specificity, the Oligo-LiveFISH loci and FISH dots were manually counted, and the number of co-localized loci were divided by the total number of Oligo-LiveFISH dots to obtain the specificity. The doublet loci, which occur in less than 5% of all loci, were excluded for localization and tracking in analysis in FIG. 6-7. FIG. 5A was generated by Matlab simulation.

### SNR and SBN Characterization

To characterize the signal-to-noise ratio (SNR) of Oligo-LiveFISH labeling, we performed a two-color Oligo-LiveFISH experiment targeting a repetitive genomic region in one color (G) and its nearby non-repetitive genomic region in a second color (R) in confocal microscope. We assumed that the repetitive loci were labeled with 100% specificity (from FISH results) and used it as the marker for the corresponding non-repetitive loci when they were imaged simultaneously. Each channel was imaged as a z-stack and a single z plane showing the highest fluorescence of the labeled loci in each channel was used to calculate the signal in that channel by a custom Matlab code. In practice, we first selected the position of the locus (x0, y0) approximately based on the image of the repetitive color channel (G). A 15-pixel×15-pixel region of interest (ROI) centered on (x0, y0) was selected for both repetitive (G) and non-repetitive (R) loci. For each channel, the central 7-pixel×7-pixel region around the peak of the loci (xl, y1) was removed and the mean signal count of the remaining 176 pixels was used as the background value. This background value was subtracted from the signal counts of all pixels of the initial 15 pixel×15 pixel ROI. The integrated signal I was calculated as a sum of these background-subtracted pixels and the signal amplitude A was calculated as

$$A = \frac{I}{2 π {σ}^{2}} \qquad ( Equation 1 )$$

using the integral of a 2D Gaussian function:

$$I = {\int }_{- ∞}^{∞} {\int }_{- ∞}^{∞} {Ae}^{- ( \frac{{( x - {x}_{0} )}^{2}}{2 {σ}_{x}^{2}} + \frac{{( y - {y}_{0} )}^{2}}{2 {σ}_{y}^{2}} )} dxdy = 2 π A {σ}_{x} {σ}_{y} = 2 π A {σ}^{2}$$

where σ is the standard deviation width of the image of the locus assuming a symmetrical Gaussian shape: σ=σₓ=σ_{y}. The signal-to-noise ratio (SNR) was then calculated using the following canonical equation:

$$SNR = \frac{Amplitude of signal}{\sqrt{( Amplitude of signal ) + ( STD of background ) \hat{} 2}} = \frac{A}{\sqrt{A + s \hat{} 2}} = \frac{\frac{I}{2 π {σ}^{2}}}{\sqrt{\frac{I}{2 π {σ}^{2}} + s \hat{} 2}} \qquad ( Equation 2 )$$

The signal-to-background-noise ratio (SBN) was calculated using the following equation:

$$SBN = \frac{Amplitude of signal}{STD of background} = \frac{A}{S} = \frac{I}{2 π {σ}^{2} s} \qquad ( Equation 3 )$$

We used σ=1.5 pixels for all non-repetitive loci and σ=1.4 pixels for all repetitive loci based on a manual measurement of the image profiles of several loci. Note that σ indicates the Gaussian width of the image of a locus, while s signifies the pixel-based standard deviation of the signal counts of the 176 background pixels from the periphery of the ROI.

### Single Particle Localization and Tracking

The centers of the DNA loci were obtained by fitting the raw images in the ThunderStorm plugin in Fiji. To obtain the position of the loci from confocal images: First, the z projection images were filtered with a wavelet filter with a b-spline order of 3 and a scale of 2. The coarse localizations were found as local maxima with an 8-neighborhood connectivity and a threshold of 2.std (Wave.F1). These localizations were weighted least squares-fitted with the integrated Gaussian model using a radius of 5 pixels and an initial sigma of 1.0 to get the (x,y) position of the chromosome loci. A 7 pixel×7 pixel region around the fitting position was selected and its intensity with different z slices were examined. The z slice with maximum intensity was selected as the z position of that locus. For two-color 2D tracking using widefield microscope, localizations were fitted to an integrated Gaussian model with maximum likelihood estimation using a radius of 8 and an initial sigma of 1.6. Tetraspeck bead data for channel registration were similarly localized with ThunderSTORM (Wavelet Filter: b-spline order 3, scale of 2; Localization Detection: local maxima, 8-neighborhood connectivity, threshold of 3·std (Wave.F1); Fitting: integrated Gaussian model, maximum likelihood estimation, radius 8, initial sigma 1.6.), and a 2D locally weighted mean quadratic transformation is computed mapping the reflected channel onto the transmitted channel. Locus localizations from the nucleus are then cropped.

Raw images of two-color 3D DHPSF tracking experiments were analyzed with the software Easy-DHPSF v.2 The software uses a model function of 2 Gaussians separated by a distance, with the axial position given by the angle formed between the line connecting both Gaussians and the horizontal. Z-stacks of the same Tetraspeck bead in each channel produced only by axial sample translation are fitted with the model function to produce a calibration curve mapping angle to z position for each color channel. Templates of the DHPSF at 8 evenly spaced angles throughout the PSFs full angular range are then chosen from the raw loci tracking data. These templates are then used to identify DHPSF signal in the raw data via phase correlation in the Fourier domain. Candidate DHPSFs are then fit with the model function using non-linear least squares fitting. Tetraspeck bead data for channel registration are similarly localized, and a 2D locally weighted mean quadratic transformation is computed mapping the reflected channel onto the transmitted channel. Chromosomal locus localizations are then filtered for lobe separation (4.5 px<lobe separation <10 px) and then registered. Z localizations are scaled by 0.7 to account for depth-dependent focal shift. If two localizations with distance <100 nm are detected in the cropped localizations, the average position of the two localizations was assigned as the localization in that frame. If the two localization has distance >100 nm or if more than 3 localizations are detected in the cropped localization, these localizations are removed.

### Single-Molecule Bleaching Experiment

To acquire single-molecule bleaching data, loci were initially imaged at a relatively low excitation power density (2 W/cm² for Cy5 and 1.4 W/cm² for Cy3) to prevent any potential photobleaching for several seconds. This data served to estimate the initial locus brightness before photobleaching. Subsequently, to observe stepwise bleaching of individual fluorophores, the power density was increased to 19 W/cm² for Cy5 or to 71 W/cm² for Cy3, and the loci were imaged until complete photobleaching occurred. Nuclei labeled with Hoechst 33342 were imaged separately at 1 W/cm². To quantify the number of fRNP bound to a single locus, we employed an approach similar to previous methods⁸⁴. Initially, an 11×11 region of interest (ROI) centered on the image of a given locus was cropped from both the low and high excitation power movies. For each frame, the background was estimated as a mean value of the 40 edge pixels of this ROI and was subtracted from the values of all pixels in the ROI. After background subtraction, a smaller 5×5 ROI was cropped from the center of the 11×11 ROI. The number of photons in each frame (the brightness) was estimated as the sum of the values of all pixels in the 5×5 ROI, considering the photoconversion gain of the camera. Plotting the brightness against time, an intensity curve was generated with discrete steps, as shown in FIG. 13A. Adapting from a previous approach⁸⁵, a semi-automated maximum likelihood change-point finding algorithm was deployed to determine the time points of each step. As each step likely represents the photobleaching of a single molecule, the difference of the averaged brightness values before and after the step was computed to estimate the brightness of a single emitter. Multiple single-molecule estimates in a given locus were further averaged to reduce noise. The brightness of the locus in the low excitation power dataset was estimated by averaging the brightness within several frames at the beginning of the low power movie. This low power brightness estimate was scaled up to the high excitation power case by using the ratio between the laser powers from the low and high power conditions. This initial locus brightness was then divided by the estimate for the brightness of a single molecule, thereby yielding the number of fRNPs bound to an individual locus. MATLAB was used to perform all calculations in this analysis.

### MSD, MSCD, VAC and VCC Analysis

The ensemble-averaged mean-square displacement (MSD) was calculated as:

$$MSD ( τ ) = 〈 {( \vec{R} ( t + τ ) - \vec{R} ( t ) )}^{2} 〉 = \frac{1}{N} {\sum }_{i} \frac{1}{m} {\sum }_{m} {[ \vec{{R}_{i}} ( mt + τ ) - {\vec{R}}_{i} ( mt ) ]}^{2} \qquad ( Equation 4 )$$

Here, N is the number of trajectories over which the ensemble average is taken. The index m runs from 1 to a specific value given the lag time τ=t_{E}×n, providing the time average of each trajectory. Here the is the exposure time and n is the number of frames spanning a time lag. The final MSD is an average over the ensemble and over time. We used the following expression for fitting the experimental MSD curves, which accounts for both static localization errors due to finite localization precision and dynamic errors due to motion blur.

$$MSD ( n ) = d \{ \frac{2 D * {t}_{E}^{α}}{( α + 2 ) ( α + 1 )} [ {( n + 1 )}^{α + 2} + {( n - 1 )}^{α + 2} - 2 {n}^{α + 2} ] - \frac{2 D * {t}_{E}^{α}}{( α + 2 ) ( α + 1 )} + 2 {σ}^{2} \} \qquad ( Equation 5 )$$

The expression was developed for anomalous diffusion consistent with fractional Brownian motion (FBM). Here d is the dimensionality, D″ is the effective diffusion coefficient, α∈(0,2] is the power law exponent of MSD with α<1 indicates subdiffusion, and 2σ² is the static error term including the spreading of the PSF due to the movement of the locus. The second to last term describes the dynamic error effects. We fitted the first 50 lags of MSDs to Equation 5 in Matlab with free parameter D*, α, and σ by minimizing the square error in the log space.

Mean square change in distance (MSCD) was calculated as

$$MSCD ( τ ) = 〈 {( Δ \vec{R} ( t + τ ) - Δ \vec{R} ( t ) )}^{2} 〉 = \frac{1}{N} {\sum }_{i} \frac{1}{m} {\sum }_{m} {[ Δ \vec{{R}_{i}} ( mt + τ ) - Δ {\vec{R}}_{i} ( mt ) ]}^{2} \qquad ( Equation 6 )$$

In Equation 6, Δ\vec{R}(t)=\vec{R}ᵢ(t)−\vec{R}ⱼ(t) is the displacement between the two loci in channel i and channel j. For velocity autocorrelation (VAC), each curve is calculated as

$${c}_{v}^{δ} ( τ ) = 〈 v ( t + τ ) \cdot v ( t ) 〉 \qquad ( Equation 7 )$$

Here δ is the time interval during which displacement occurred for calculating the velocity:

$$v ( t ) = \frac{1}{δ} ( r ( t + δ ) - r ( t ) ) \qquad ( Equation 8 )$$

And the τ is the time lag over which the correlation of velocities is examined. The normalized discrete velocity autocorrelation function for fractional Brownian motion (FBM) is

$$\frac{{c}_{v}^{δ} ( τ )}{{c}_{v}^{δ} ( 0 )} = \frac{{\vert τ - δ \vert}^{α} + {\vert τ + δ \vert}^{α} - 2 {\vert τ \vert}^{α}}{2 {δ}^{α}} \qquad ( Equation 9 )$$

For velocity cross-correlation (VCC) of two sites i and j, each curve is calculated as

$${c}_{vv}^{δ} ( τ ) = 〈 vi ( t + τ ) \cdot vj ( t ) 〉 \qquad ( Equation 10 )$$

δ is the time interval during which displacement occurred for calculating the velocity for each channel:

$$vi ( t ) = \frac{1}{δ} ( ri ( t + δ ) - ri ( t ) ) \qquad ( Equation 11 )$$

And the τ is the time lag over which the correlation of velocities is examined.

### Polymer Modeling

We applied a viscoelastic coarse-grained Rouse polymer model developed before to fit Oligo-LiveFISH tracking data. Briefly, the polymer chain is discretized into monomer segments which experience hydrodynamic drag and are connected into a linear chain by Hookean springs. The spring coefficient between the monomers is k=3k_{B}Tb⁻², where kB is the Boltzmann constant, T is the temperature and b is the Kuhn length. In our viscoelastic Rouse polymer model, each monomer exhibits a drag coefficient ξ and a viscoelastic response to motion that is mediated by the memory kernel

K(t)=(2−α′)(1−α′)|t|^{-α}′ (Equation 12)

α′ refers to the power-law exponent associated with the MSD of a single particle in viscoelastic medium and equals to 2 α, where α is the MSD power-law exponent for the polymer as defined in Eq. 5. By fitting experimental MSD using Eq.5, we got α=0.43 and 0.48 for site 1 and 2 and we chose a mean α=0.45. Therefore, we assign α′=2 α=0.9. The dynamic motion of polymer is governed by the fractional Langevin equation:

$$ξ {\int }_{0}^{t} {dt}^{′} K ( t - {t}^{′} ) \frac{\partial \overline{R} ( n , {t}^{′} )}{\partial t} = k \frac{{\partial }^{2} \overline{R} ( n , t )}{\partial {n}^{2}} + \vec{F} ( n , t ) \qquad ( Equation 13 )$$

where the left-hand term indicates contributions from viscoelastic drag described by the memory kernel K(t−t′), first right-hand term denotes Gaussian springs with spring constant k and, \vec{F}(n, t) denotes Brownian force that obeys the fluctuation-dissipation theorem. For a finite polymer chain of N Kuhn segments, the Rouse time TR is given by

$${τ}_{R} = {[ \frac{{N}^{2} {b}^{2} ξ}{{k}_{B} T} ]}^{\frac{1}{{a}^{′}}} \qquad ( Equation 14 )$$

and in the intermediate regime where & is large enough for stress to communicate between two sites but small enough that it has not reach the chain ends, τ_{R}(Δn), can be approximated as

$${τ}_{R} ( Δ n ) = {[ \frac{{( Δ n )}^{2} {b}^{2} ξ}{{k}_{B} T} ]}^{\frac{1}{{a}^{′}}} \qquad ( Equation 15 )$$

and in the intermediate regime where δ is large enough for stress to communicate between two sites but small enough that it has not reach the chain ends, τ_{R}(Δn), can be approximated as

$$\frac{δ}{{τ}_{R} ( Δ n )} = \frac{δ}{{τ}_{R}} {( \frac{\vert Δ n \vert}{N} )}^{\frac{2}{{a}^{′}}} \qquad ( Equation 16 )$$

By generating a series of theoretical VCC using τ_{R}(Δn) as variable and find the VCC which has lowest mean squared error (MSE) with respect to the experimental VCC, we find τ_{R}(Δn) for different sites. An MSE is calculated for each δ and τ_{R}(Δn) separately and these MSEs are averaged to obtain the final MSE to be minimized. For sites 1&2, τ_{R}(Δn) was examined from 0.01 s to 10 s with 0.01 s increments. For sites 2&3 and 3&4, τ_{R}(Δn) was examined from Is to 5000 s with 1 s increments. The predicted τ_{R}(Δn) from polymer theory can be calculated from Eq15 using τ_{R}(Δn=28 kb)=0.44 s as a reference. For example, τ_{R}

$${τ}_{R} ( Δ n = 284 kb ) = {( \frac{284 kb}{28 kb} )}^{\frac{2}{0.9}} \times 0.44 s = 76 s ,$$

which is very close to the 89 s by fitting experimental VCC. On the other hand, the effective genomic length Δn can also be calculated using Eq 15 by inputting experimental τ_{R}(Δn) as known variable (data not shown).
Spatial Contact and Looping Probability from Hi-C

Spatial interaction between sites were generated with Juicebox using vanilla coverage square root (VC_SQRT) normalized Hi-C signal (GSM4194449) at 50 Kb resolution. Hi-C was plotted using Juicebox. The relationship between genome-averaged contact probability P(s) and genomic distance was adopted from a previously simulations with steady-state extrusion. The spatial contacts from Hi-C were normalized to match the contact probability P(s) using sites 1 & 2 as a reference.

### Cluster Analysis for the Pair-Wise Inter-Locus 3D Distance

A Gaussian Mixture Model (GMM) was applied to identify clusters in FIG. 14C by assuming that all data points are drawn from a probability density function composed of k independent Gaussian components. We fit the GMM parameters using the built-in Matlab function “fitgmdist”, which uses an expectation maximization algorithm to determine maximum likelihood parameters based on the observed data (1000 max iterations). We repeated this for k=1 to k=10 and selected the final model (k=2) based on the minimum value of the Bayesian Information Criterion (BIC), which introduces a penalty for each additional term to prevent overfitting. For each point, the posterior probability of being a member of a cluster was calculated using the Matlab function “posterior” and the data points were assigned to the clusters with higher membership probability (>0.5).

### Machine Learning to Investigate the Key Parameters

We used experimentally calculated SNR as predictors in a linear regression model:

SNR=β₀+×β^{T}+ε (Equation 17)
x: =(number of probes, GC content, crRNA length, crRNA structure, distance to H3K27ac peak, ATAC, H3K4me3, H3K4mel). β: =(B₁, . . . , βₖ)^{T}, k=8.

The regression model is fitted using the glmnet R package (version 4.1-8) by elastic net regularization with the a set to 0.5, and the penalty parameter 2 which is given by the model with the minimum mean squared error in cross-validation curve. We first examined 15 features with nonzero coefficients including: number of distinct gRNA species (12, 24, 48, 96 or 192), the GC percentage of the spacer sequence, the average length of crRNA, the average secondary structure scores of crRNAs, the average secondary structure scores of crRNA: tracrRNA duplexes, the average distance of the target regions to the closest H3K27ac peak, the average distance of the target regions to the closest ATAC peak, average H3K27ac signal, average ATAC signal, average H3K4mel signal, average H3K18ac signal, average H3K56ac signal, average H3K9ac signal, average H3K9me3 signal, and average H3K4me3 signal. We performed correlation analysis and removed the highly-correlative variables and the selected 8 features: number of distinct gRNA species, the average GC percentage in the spacer sequence, the average length of crRNA, the average secondary structure scores of crRNAs, the average distance of the target regions to the closest H3K27ac peak, average ATAC signal, average H3K4mel signal, and average H3K4me3 signal. The average secondary structure scores of crRNAs were calculated by RNAfold [--gquad] on the crRNA sequences and the crRNA: tracrRNA duplexes. The model was selected by 10-fold cross-validation.

We randomly selected two samples as test set and remaining 17 samples as training set. We used the training set for parameter selection and model training. Finally, we used the model to predict the variable and calculate the square error according to the real value. To evaluate the performance of the model, we compute the squared errors 100 times and the average square error is smaller for the model trained by selected variables compared to the model trained by all variables.

### Statistics Analysis

For FIG. 2B, 10B-10F, the p values were calculated using two-tailed unpaired t test in Graph Pad. For FIG. 10C-10F, the Peason correlation coefficient r were calculated in Graph Pad. For FIG. 2G, 2H, 11C, 11K, 11L, the Peason correlation coefficient r and its significance level (p value) were calculated in R.

### Data and Code Availability

Source data for all figures are available online. Custom scripts used for image analysis are available at Github.com (github (.) com/QilabGitHub) and upon request.

### REFERENCES

1. Zheng, H.; Xie, W., The role of 3D genome organization in development and cell differentiation. Nature Reviews Molecular Cell Biology 2019, 20 (9), 535, DOI: 10.1038/s41580-019-0132-4.
2 Hnisz, D., et al., Activation of proto-oncogenes by disruption of chromosome neighborhoods. Science 2016, 351 (6280), 1454, DOI: 10.1126/science.aad9024.
3. Lupianez, D. G., et al., Disruptions of topological chromatin domains cause pathogenic rewiring of gene-enhancer interactions. Cell 2015, 161 (5), 1012, DOI: 10.1016/j.cell.2015.04.004.
4. Beagan, J. A., et al., YY1 and CTCF orchestrate a 3D chromatin looping switch during early neural lineage commitment. Genome Res 2017, DOI: 10.1101/gr.215160.116.
5. Waldman, T., Emerging themes in cohesin cancer biology. Nat Rev Cancer 2020, 20 (9), 504, DOI: 10.1038/s41568-020-0270-1.
6. Weintraub, A. S., et al., YY1 Is a Structural Regulator of Enhancer-Promoter Loops. Cell 2017, 171 (7), 1573, DOI: 10.1016/j.cell.2017.11.008.
7. Sanyal, A., et al., The long-range interaction landscape of gene promoters. Nature 2012, 489 (7414), 109, DOI: 10.1038/nature11279
8. Javierre, B. M., et al., Lineage-Specific Genome Architecture Links Enhancers and Non-coding Disease Variants to Target Gene Promoters. Cell 2016, 167 (5), 1369, DOI: 10.1016/j.cell.2016.09.037.
9. Bintu, B., et al., Super-resolution chromatin tracing reveals domains and cooperative interactions in single cells. Science 2018, 362 (6413), 419.
10. Dekker, J., et al., Exploring the three-dimensional organization of genomes: interpreting chromatin interaction data. Nat Rev Genet 2013, 14 (6), 390, DOI: 10.1038/nrg3454.
11. Dixon, J. R., et al., Topological domains in mammalian genomes identified by analysis of chromatin interactions. Nature 2012, 485 (7398), 376.
12. Wang, S. Y., et al., Spatial organization of chromatin domains and compartments in single chromosomes. Science 2016, 353 (6299), 598, DOI: 10.1126/science.aaf808.
13. Lakadamyali, M.; Cosma, M. P., Visualizing the genome in high resolution challenges our textbook understanding. Nature Methods 2020, 17 (4), 371.
14. Lieberman-Aiden, Comprehensive mapping of long-range interactions reveals folding principles of the Human Genome. Science 2009, 326, 289, DOI: 10.1126/science. 1181369.
15. Roukos, V., et al., Spatial dynamics of chromosome translocations in living cells. Science 2013, 341 (6146), 660, DOI: 10.1126/science.1237150.
16. Chen, B., et al., Imaging Specific Genomic DNA in Living Cells. Annu Rev Biophys 2016, 45, 1, DOI: 10.1146/annurev-biophys-062215-010830.
17. Brandao, H. B., et al., Tracking and interpreting long-range chromatin interactions with super-resolution live-cell imaging. Current Opinion in Cell Biology 2021, 70, 18, DOI: 10.1016/j.ceb.2020.11.002.
18. Robinett, C. C., et al., In vivo localization of DNA sequences and visualization of large-scale chromatin organization using lac operator/repressor recognition. J Cell Biol 1996, 135 (6 Pt 2), 1685, DOI: 10.1083/jcb. 135.6.1685.
19. Ghosh, R. P., et al., A fluorogenic array for temporally unlimited single-molecule tracking. Nat Chem Biol 2019, 15 (4), 401, DOI: 10.1038/s41589-019-0241-6.
20. Chen, B. H., et al., Dynamic Imaging of Genomic Loci in Living Human Cells by an Optimized CRISPR-Cas System. Cell 2013, 155 (7), 1479.
21. Ma, H. H., et al., Multiplexed labeling of genomic loci with dCas9 and engineered sgRNAs using CRISPRainbow. Nature Biotechnology 2016, 34 (5), 528.
22. Ma, H., et al., CRISPR-Sirius: RNA scaffolds for signal amplification in genome imaging. Nat Methods 2018, 15 (11), 928, DOI: 10.1038/s41592-018-0174-0.
23. Tanenbaum, M. E., et al., A protein-tagging system for signal amplification in gene expression and fluorescence imaging. Cell 2014, 159 (3), 635, DOI: 10.1016/j.cell.2014.09.039.
24. Shao, S., et al., Long-term dual-color tracking of genomic loci by modified sgRNAs of the CRISPR-Cas9 system. Nucleic Acids Res 2016, 44 (9), e86, DOI: 10.1093/nar/gkw066.
25. Wu, X., et al., A CRISPR/molecular beacon hybrid system for live-cell genomic imaging. Nucleic Acids Res 2018, 46 (13), e80, DOI: 10.1093/nar/gky304.
7. Wang, H., et al., CRISPR-mediated live imaging of genome editing and transcription. Science 2019, 365 (6459), 1301, DOI: 10.1126/science.aax7852.
8. Qin, P. W., et al., Live cell imaging of low- and non-repetitive chromosome loci using CRISPR-Cas9. Nature Communications 2017, 8.
9. Lyu, X. Y., et al., CRISPR FISHer enables high-sensitivity imaging of nonrepetitive DNA in living cells through phase separation-mediated signal amplification. Cell Res 2022, 32 (11), 969, DOI: 10.1038/s41422-022-00712-z.
10. Gu, B., et al., Transcription-coupled changes in nuclear mobility of mammalian cis-regulatory elements. Science 2018, 359 (6379), 1050, DOI: 10.1126/science.aao3136.
11. Geng, Y. T.; Pertsinidis, A., Simple and versatile imaging of genomic loci in live mammalian cells and early pre-implantation embryos using CAS-LiveFISH. Scientific Reports 2021, 11 (1), DOI: ARTN 12220 10.1038/s41598-021-91787-y.
12. Clow, P. A., et al., CRISPR-mediated multiplexed live cell imaging of nonrepetitive genomic loci with one guide RNA per locus. Nature Communications 2022, 13 (1), DOI: ARTN 1871 10.1038/s41467-022-29343-z.
13. Zhou, Y. X., et al., Painting a specific chromosome with CRISPR-Cas9 for live-cell imaging. Cell Research 2017, 27 (2), 298, DOI: 10.1038/cr.2017.9.
14. Van Tricht, C., et al., Imaging the unimaginable: leveraging signal generation of CRISPR-Cas for sensitive genome imaging. Trends Biotechnol 2023, 41 (6), 769, DOI: 10.1016/j.tibtech.2022.10.003.
15. Jinek, M., et al., A Programmable Dual-RNA-Guided DNA Endonuclease in Adaptive Bacterial Immunity. Science 2012, 337 (6096), 816.
16. Anhauser, L., et al., Multiple covalent fluorescence labeling of eukaryotic mRNA at the poly(A) tail enhances translation and can be performed in living cells. Nucleic Acids Res 2019, 47 (7), e42, DOI: 10.1093/nar/gkz084.
17. Lin, X., et al., Nested epistasis enhancer networks for robust genome regulation. Science 2022, 377 (6610), 1077, DOI: 10.1126/science.abk3512.
18. Xu, H. Y., et al., TriTag: an integrative tool to correlate chromatin dynamics and gene expression in living cells (vol 48, pg e127, 2020). Nucleic Acids Research 2020, 48 (22), 13013.
19. Chen, B., et al., Efficient labeling and imaging of protein-coding genes in living cells using CRISPR-Tag. Nat Commun 2018, 9 (1), 5065, DOI: 10.1038/s41467-018-07498-y.
20. Yang, L. Z., et al., CRISPR-array-mediated imaging of non-repetitive and multiplex genomic loci in living cells. Nat Methods 2024, DOI: 10.1038/s41592-024-02333-3.
21. Qi, L. S., et al., Repurposing CRISPR as an RNA-Guided Platform for Sequence-Specific Control of Gene Expression. Cell 2013, 152 (5), 1173, DOI: 10.1016/j.cell.2013.02.022.
22. Akan, P., et al., Comprehensive analysis of the genome transcriptome and proteome landscapes of three tumor cell lines. Genome Med 2012, 4 (11), 86, DOI: 10.1186/gm387.
23. Greenberg, M. E.; Ziff, E. B., Stimulation of 3t3 Cells Induces Transcription of the C-Fos Proto-Oncogene. Nature 1984, 311 (5985), 433, DOI: https://doi.org/10.1038/311433a0.
24. Yap, E. L.; Greenberg, M. E., Activity-Regulated Transcription: Bridging the Gap between Neural Activity and Behavior. Neuron 2018, 100 (2), 330, DOI: 10.1016/j.neuron.2018.10.013.
25. Beagan, J. A., et al., Three-dimensional genome restructuring across timescales of activity-induced neuronal gene expression. Nature Neuroscience 2020, 23 (6), 707, DOI: 10.1038/s41593-020-0634-6.
26. Sato, H., et al., Imaging of DNA and RNA in Living Eukaryotic Cells to Reveal Spatiotemporal Dynamics of Gene Expression. Annu Rev Biochem 2020, 89, 159, DOI: 10.1146/annurev-biochem-011520-104955.
27. Kempfer, R.; Pombo, A., Methods for mapping 3D chromosome architecture. Nat Rev Genet 2020, 21 (4), 207, DOI: 10.1038/s41576-019-0195-2.
28. Dekker, J., et al., The 4D nucleome project. Nature 2017, 549 (7671), 219.
29. Mockl, L.; Moerner, W. E., Super-resolution Microscopy with Single Molecules in Biology and Beyond-Essentials, Current Trends, and Future Challenges. J Am Chem Soc 2020, 142 (42), 17828, DOI: 10.1021/jacs.0c08178.
49. von Diezmann, L., et al., Three-Dimensional Localization of Single Molecules for Super-Resolution Imaging and Single-Particle Tracking. Chem Rev 2017, 117 (11), 7244, DOI: 10.1021/acs.chemrev.6b00629.
30. Gelles, J., et al., Tracking kinesin-driven movements with nanometre-scale precision. Nature 1988, 331 (6155), 450, DOI: 10.1038/331450a0.
31. Gabriele, M., et al., Dynamics of CTCF- and cohesin-mediated chromatin looping revealed by live-cell imaging. Science 2022, 376 (6592), 496, DOI: 10.1126/science.abn6583.
32. Knight, S. C., et al., Dynamics of CRISPR-Cas9 genome interrogation in living cells. Science 2015, 350 (6262), 823.
33. Gustavsson, A. K., et al., Fast and parallel nanoscale three-dimensional tracking of heterogeneous mammalian chromatin dynamics. Mol Biol Cell 2022, 33 (6), ar47, DOI: 10.1091/mbc.E21-10-0514.
34. Pavani, S. R., et al., Three-dimensional, single-molecule fluorescence imaging beyond the diffraction limit by using a double-helix point spread function. Proc Natl Acad Sci USA 2009, 106 (9), 2995, DOI: 10.1073/pnas.0900245106.
35. Backlund, M. P., et al., Chromosomal locus tracking with proper accounting of static and dynamic errors. Phys Rev E Stat Nonlin Soft Matter Phys 2015, 91 (6), 062716, DOI: 10.1103/PhysRevE.91.062716.
36. Weber, S. C., et al., Analytical tools to distinguish the effects of localization error, confinement, and medium elasticity on the velocity autocorrelation function. Biophys J 2012, 102 (11), 2443, DOI: 10.1016/j.bpj.2012.03.062.
37. Weber, S. C., et al., Bacterial chromosomal loci move subdiffusively through a viscoelastic cytoplasm. Phys Rev Lett 2010, 104 (23), 238102, DOI: 10.1103/PhysRevLett. 104.238102.
38. Zhu, Y., et al., Rigidification of the Escherichia coli cytoplasm by the human antimicrobial peptide LL-37 revealed by superresolution fluorescence microscopy. Proceedings of the National Academy of Sciences of the U.S. Pat. No. 2,019,116 (3), 1017, DOI: 10.1073/pnas.1814924116.
39. Zhu, Y., et al., Biophysical Properties of Escherichia coli Cytoplasm in Stationary Phase by Superresolution Fluorescence Microscopy. mBio 2020, 11 (3), DOI: 10.1128/mBio.00143-20.
40. Zhang, Y.; Dudko, O. K., First-Passage Processes in the Genome. Annu Rev Biophys 2016, 45, 117, DOI: 10.1146/annurev-biophys-062215-010925.
41. Lucas, J. S., et al., 3D trajectories adopted by coding and regulatory DNA elements: first-passage times for genomic interactions. Cell 2014, 158 (2), 339, DOI: 10.1016/j.cell.2014.05.036.
42. Backlund, M. P., et al., Correlations of three-dimensional motion of chromosomal loci in yeast revealed by the double-helix point spread function microscope. Mol Biol Cell 2014, 25 (22), 3619, DOI: 10.1091/mbc.E14-06-1127.
43. Lampo, T. J., et al., Physical Modeling of Dynamic Coupling between Chromosomal Loci. Biophysical Journal 2016, 110 (2), 338, DOI: 10.1016/j.bpj.2015.11.3520.
44. Squires, A. H.; Moerner, E., Direct single-molecule measurements of phycocyanobilin photophysics in monomeric C-phycocyanin. Proc Natl Acad Sci USA 2017, 114 (37), 9779, DOI: 10.1073/pnas. 1705435114.
45. Squires, A. H., et al., Single-molecule trapping and spectroscopy reveals photophysical heterogeneity of phycobilisomes quenched by Orange Carotenoid Protein. Nat Commun 2019, 10 (1), 1172, DOI: 10.1038/s41467-019-09084-2.
46. Mirny, L. A., The fractal globule as a model of chromatin architecture in the cell. Chromosome Res 2011, 19 (1), 37, DOI: 10.1007/s10577-010-9177-0.
47. Bruckner, D. B., et al., Stochastic motion and transcriptional dynamics of pairs of distal DNA loci on a compacted chromosome. Science 2023, 380 (6652), 1357, DOI: 10.1126/science.adf5568.
48. Chan, B.; Rubinstein, M., Activity-driven chromatin organization during interphase: Compaction, segregation, and entanglement suppression. Proc Natl Acad Sci USA 2024, 121 (21), e2401494121, DOI: 10.1073/pnas.2401494121.
49. Wu, X., et al., Progress and Challenges for Live-cell Imaging of Genomic Loci Using CRISPR-based Platforms. Genomics Proteomics Bioinformatics 2019, 17 (2), 119, DOI: 10.1016/j.gpb.2018.10.001.
50. Saurabh, S., et al., Super-resolution Imaging of Live Bacteria Cells Using a Genetically Directed, Highly Photostable Fluoromodule. J Am Chem Soc 2016, 138 (33), 10398, DOI: 10.1021/jacs.6b05943.
71. Gu, B., et al., Opposing Effects of Cohesin and Transcription on CTCF Organization Revealed by Super-resolution Imaging. Mol Cell 2020, 80 (4), 699, DOI: 10.1016/j.molcel.2020.10.001.
51. Gao, Y., et al., Complex transcriptional modulation with orthogonal and inducible dCas9 regulators. Nature Methods 2016, 13 (12), 1043, DOI: 10.1038/NMETH.4042.
52. Fernandopulle, M. S., et al., Transcription Factor-Mediated Differentiation of Human iPSCs into Neurons. Curr Protoc Cell Biol 2018, 79 (1), e51, DOI: 10.1002/cpcb.51.
53. Tieu, V., et al., A versatile CRISPR-Cas13d platform for multiplexed transcriptomic regulation and metabolic engineering in primary human T cells. Cell 2024, 187 (5), 1278, DOI: 10.1016/j.cell.2024.01.035.
54. Bae, S., et al., Cas-OFFinder: a fast and versatile algorithm that searches for potential off-target sites of Cas9 RNA-guided endonucleases. Bioinformatics 2014, 30 (10), 1473, DOI: 10.1093/bioinformatics/btu048.
55. Barrett, T., et al., NCBI GEO: archive for functional genomics data sets--update. Nucleic Acids Res 2013, 41 (Database issue), D991, DOI: 10.1093/nar/gks1193.
56. Zhang, Y., et al., Rapid single-step induction of functional neurons from human pluripotent stem cells. Neuron 2013, 78 (5), 785, DOI: 10.1016/j.neuron.2013.05.029.
57. Nehme, R., et al., Combining NGN2 Programming with Developmental Patterning Generates Human Excitatory Neurons with NMDAR-Mediated Synaptic Transmission. Cell Rep 2018, 23 (8), 2509, DOI: 10.1016/j.celrep.2018.04.066.
58. Roy, A. R., et al., Exploring Cell Surface-Nanopillar Interactions with 3D Super-Resolution Microscopy. ACS Nano 2022, 16 (1), 192, DOI: 10.1021/acsnano. 1c05313.
59. Andronov, L., et al., Nanoscale cellular organization of viral RNA and proteins in SARS-CoV-2 replication organelles. Nat Commun 2024, 15 (1), 4644, DOI: 10.1038/s41467-024-48991-x.
60. Gahlmann, A., et al., Quantitative multicolor subdiffraction imaging of bacterial protein ultrastructures in three dimensions. Nano Lett 2013, 13 (3), 987, DOI: 10.1021/n1304071h.
61. Schindelin, J., et al., Fiji: an open-source platform for biological-image analysis. Nat Methods 2012, 9 (7), 676, DOI: 10.1038/nmeth.2019.
62. Ovesny, M., et al., ThunderSTORM: a comprehensive ImageJ plug-in for PALM and STORM data analysis and super-resolution imaging. Bioinformatics (Oxford, England) 2014, 30 (16), 2389, DOI: 10.1093/bioinformatics/btu202 [doi].
63. Jiang, Y., et al., Sensing cooperativity in ATP hydrolysis for single multisubunit enzymes in solution. Proc Natl Acad Sci USA 2011, 108 (41), 16962, DOI: 10.1073/pnas.1112244108.
64. Squires, A. H., et al., Interferometric Scattering Enables Fluorescence-Free Electrokinetic Trapping of Single Nanoparticles in Free Solution. Nano Lett 2019, 19 (6), 4112, DOI: 10.1021/acs.nanolett.9b01514.
65. Zhu, Y., et al., Local rigidification and possible coacervation of the Escherichia coli DNA by cationic nylon-3 polymers. Biophys J 2021, 120 (23), 5243, DOI: 10.1016/j.bpj.2021.10.037.
66. Newman, T. A. C., et al., Diffusion and distal linkages govern interchromosomal dynamics during meiotic prophase. Proc Natl Acad Sci USA 2022, 119 (12), e2115883119, DOI: 10.1073/pnas.2115883119.

1. A method for generating a list of chromatin accessible target sequences for CRISPR-Cas guide RNAs that target a contiguous genomic region, the method comprising using one or more computer processors to:
(1) generate a master list of all possible target sequences in a genomic region of interest for a PAM sequence of interest, wherein each target sequence is 13-20 nucleotides in length; and
(2) generate a list of chromatin accessible target sequences from said master list, via:
(i) removing any target sequence from the master list that would result in any of the following sequences being contained in a CRISPR-Cas guide RNA that targets said target sequence: UUUU; (G) 10, CUCGAG, GCUNAGC, or CCANNNNNNUGG;
(ii) removing any target sequence from the master list that has more than 100 off-target sites, wherein an off-target site is a sequence outside of the genomic region of interest that is adjacent the PAM sequence of interest and:
(a) has 0 or 1 mismatch to the target sequence for target sequences that are less than 16 nucleotides in length; or
(b) has 0, 1, or 2 mismatches to the target sequence for target sequences that are more than 15 nucleotides in length;
(iii) removing any target sequence from the master list that has an off-target site in an off-target region, wherein an off-target region is a genomic region outside the genomic region of interest that:
(a) is less than 2000 nucleotides in length; and
(b) contains two or more off-target sites as defined in (2) (ii);
(iv) after performing (i), (ii), and (iii): removing, in 5′ to 3′ order along the genomic region of interest, any remaining target sequence from the master list that is 3′ of, and within 10 nucleotides of, another remaining target sequence of the master list; and
(v) selecting chromatin accessible target sequences from the remaining target sequences of the master list by:
(a) providing one or more histone modification and/or ATAC-seq datasets corresponding to the genomic region of interest, wherein the one or more histone modification datasets comprise H3K27 acetylation enrichment peak data; and
(b) selecting target sequences in the list within 5000 nucleotides in distance from an H3K27 acetylation peak and/or an ATAC-seq peak for inclusion in the list of target sequences,
thereby generating a list of chromatin accessible target sequences for CRISPR-Cas guide RNAs that target a genomic region of interest.

2. The method of any of the preceding claims, wherein the genomic region of interest is a non-repetitive genomic region.

3. The method of claim 1 or 2, wherein the genomic region of interest is at least 500 contiguous nucleotides in length.

4. The method of claim 3, wherein the genomic region of interest is 500 to 2,000,000 contiguous nucleotides in length.

5. The method of any one of claims 3-4, wherein the method generates a list of at least 3 chromatin accessible target sequences.

6. The method of any one of claims 3-5, wherein the method generates a list of 3 to 20,000 chromatin accessible target sequences.

7. The method of any one of claims 3-6, wherein the method generates a list of one chromatin accessible target sequence for every 100 bp-150 bp in the genomic region of interest.

8. The method of any one of claims 1-7, wherein the method further comprises producing a plurality of CRISPR-Cas guide RNAs that target sequences from the list of chromatin accessible target sequences.

9. The method of claim 8, wherein the producing comprises in vitro transcribing the plurality of CRISPR-Cas guide RNAs from a plurality of template nucleic acids.

10. The method of claim 9, wherein the template nucleic acids are linear single-stranded oligonucleotides.

11. A plurality of in vitro transcribed CRISPR-Cas guide RNAs, wherein each guide RNA comprises:
(a) a guide sequence that hybridizes to a target sequence; and
(b) a scaffold sequence that can bind to a CRISPR-Cas effector protein,
wherein the plurality comprises 3 or more of said CRISPR-Cas guide RNAs that hybridize to different target sequences within a genomic region of interest at least 500 contiguous nucleotides in length.

12. The plurality of in vitro transcribed CRISPR-Cas guide RNAs of claim 11, wherein the plurality comprises 3 to 20,000 of said CRISPR-Cas guide RNAs that hybridize to different target sequences within the genomic region of interest.

13. The plurality of in vitro transcribed CRISPR-Cas guide RNAs of claim 12, wherein the plurality comprises 20 to 20,000 of said CRISPR-Cas guide RNAs that hybridize to different target sequences within the genomic region of interest.

14. The plurality of in vitro transcribed CRISPR-Cas guide RNAs of any one of claims 11-13, wherein the genomic region of interest is 500 to 2,000,000 contiguous nucleotides in length.

15. The plurality of in vitro transcribed CRISPR-Cas guide RNAs of any one of claims 11-14, wherein the genomic region of interest is a non-repetitive genomic region.

16. The plurality of in vitro transcribed CRISPR-Cas guide RNAs of any one of claims 11-15, wherein said guide RNAs target sequences selected from a list of chromatin accessible target sequences generated according to the method of any one of claims 1-7.

17. The plurality of in vitro transcribed CRISPR-Cas guide RNAs of any one of claims 11-16, wherein the guide RNAs are single-molecule guide RNAs such that (a) and (b) are present on the same nucleic acid molecule.

18. The plurality of in vitro transcribed CRISPR-Cas guide RNAs of any one of claims 11-16, wherein the guide RNAs are not single-molecule guide RNAs such that (a) and (b) are present on different nucleic acid molecules.

19. The plurality of in vitro transcribed CRISPR-Cas guide RNAs of claim 17, wherein the guide RNAs comprise a 3′ poly(A) tail, wherein at least one adenosine of the poly(A) tail comprises a heterologous moiety.

20. The plurality of in vitro transcribed CRISPR-Cas guide RNAs of claim 17 or claim 19, wherein the guide RNAs comprise a heterologous moiety at their 5′ end.

21. The plurality of in vitro transcribed CRISPR-Cas guide RNAs of claim 18 or 20, wherein the 5′ end of the nucleic molecule that comprises (a), and/or the 5′ end of the nucleic molecule that comprises (b), comprises a heterologous moiety.

22. The plurality of in vitro transcribed CRISPR-Cas guide RNAs of claim 18 or 20, wherein the nucleic molecule that comprises (a), and/or the nucleic molecule that comprises (b), comprises a 3′ poly(A) tail, wherein at least one adenosine of the poly(A) tail comprises a heterologous moiety.

23. The plurality of in vitro transcribed CRISPR-Cas guide RNAs of any one of claims 19-22, wherein the heterologous moiety comprises a fluorescent dye.

24. The plurality of in vitro transcribed CRISPR-Cas guide RNAs of claim 23, wherein the fluorescent dye is selected from: Cy3, Cy5, A488, A565, A647, and any derivatives thereof.

25. The plurality of in vitro transcribed CRISPR-Cas guide RNAs of any one of claims 11-24, wherein the CRISPR-Cas effector protein is a Class 2 CRISPR-Cas effector protein.

26. The plurality of in vitro transcribed CRISPR-Cas guide RNAs of claim 25, wherein the Class 2 CRISPR-Cas effector protein is a Type II or Type V CRISPR-Cas effector protein.

27. A CRISPR-Cas guide RNA comprising:
(a) a guide sequence that hybridizes to a target sequence;
(b) a scaffold sequence that can bind to a CRISPR-Cas effector protein; and
(c) a 3′ poly(A) tail, wherein at least one adenosine of the poly(A) tail comprises a heterologous moiety.

28. The CRISPR-Cas guide RNA of claim 27, wherein the guide RNA is a single-molecule guide RNA such that (a) and (b) are present on the same nucleic acid molecule.

29. The CRISPR-Cas guide RNA of claim 27, wherein the guide RNA is not a single-molecule guide RNA such that (a) and (b) are present on different nucleic acid molecules.

30. The CRISPR-Cas guide RNA of claim 29, wherein the nucleic acid that comprises (b) comprises the 3′ poly(A) tail.

31. The CRISPR-Cas guide RNA of any one of claims 27-30, where said at least one adenosine is an azido-modified adenosine.

32. The CRISPR-Cas guide RNA of any one of claims 27-31, wherein the heterologous moiety comprises a fluorescent dye.

33. The CRISPR-Cas guide RNA of claim 32, wherein the fluorescent dye is selected from: Cy3, Cy5, A488, A565, A647, and any derivatives thereof.

34. The CRISPR-Cas guide RNA of any one of claims 27-33, wherein the CRISPR-Cas effector protein is a Class 2 CRISPR-Cas effector protein.

35. The CRISPR-Cas guide RNA of claim 34, wherein the Class 2 CRISPR-Cas effector protein is a Type II or Type V CRISPR-Cas effector protein.

36. The CRISPR-Cas guide RNA of any one of claims 27-35, wherein said guide RNAs target a target sequence selected from a list of chromatin accessible target sequences generated according to the method of any one of claims 1-7.

37. A method of labeling a CRISPR-Cas guide RNA, the method comprising: contacting a CRISPR-Cas guide RNA with a poly(A) polymerase and adenosine triphosphate (ATP) molecules, wherein at least one ATP molecule comprises a heterologous moiety, thereby generating a CRISPR-Cas guide RNA comprising a poly(A) tail in which at least one adenosine of the poly(A) tail comprises the heterologous moiety.

38. The method of claim 37, wherein the heterologous moiety is an azide moiety.

39. The method claim 37 or 38, wherein the method further comprises, conjugating a fluorescent dye to the heterologous moiety.

40. The method of claim 39, wherein the fluorescent dye comprises an azadibenzylcyclooctyne (DBCO) moiety.

41. The method of any one of claims 39-40, wherein the conjugation is performed via Click chemistry.

42. The method of any one of claims 39-41, wherein the fluorescent dye is selected from: Cy3, Cy5, A488, A565, A647, and any derivatives thereof.

43. A plurality of guide RNA template nucleic acids, each template nucleic acid comprising:
(a) a first primer binding domain common to a subset of the plurality of template nucleic acids;
(b) an RNA-polymerase promoter; and
(c) a sequence encoding a guide RNA, wherein the guide RNA comprises:
(i) a guide sequence that hybridizes to a target sequence; and
(ii) a sequence common to the plurality of template nucleic acids,
wherein the guide sequences encoded in the template nucleic acids hybridize to target sequences within a genomic region of interest at least 500 contiguous nucleotides in length.

44. The plurality of guide RNA template nucleic acids of claim 43, wherein the sequence common to the plurality of template nucleic acids of (c) (ii) comprises a scaffold sequence that can bind to a CRISPR-Cas effector protein.

45. The plurality of guide RNA template nucleic acids of claim 43 or 44, wherein the plurality comprises 3 to 20,000 of said template nucleic acids.

46. The plurality of guide RNA template nucleic acids of any one of claims 43-45, wherein the guide sequences encoded in the template nucleic acids hybridize to a target sequence within a genomic region of interest 500 to 2,000,000 contiguous nucleotides in length.

47. The plurality of guide RNA template nucleic acids of any one of claims 43-46, wherein said guide RNAs target sequences selected from a list of chromatin accessible target sequences generated according to the method of any one of claims 1-7.

48. The plurality of guide RNA template nucleic acids of any one of claims 43-47, wherein the template nucleic acids are linear single-stranded oligonucleotides.

49. A method of preparing a plurality of CRISPR-Cas guide RNAs for targeting a contiguous genomic region, the method comprising:
(a) contacting a plurality of template nucleic acids according to any one of claims 43-48, or a subset thereof, with an RNA polymerase; and
(b) transcribing the plurality of template nucleic acids, or a subset thereof, to produce a plurality of in vitro transcribed CRISPR-Cas guide RNAs for targeting a contiguous genomic region.

50. The method of claim 49, wherein the method further comprises, prior to said contacting of (a): contacting the template nucleic acid composition with a forward primer and a reverse primer to amplify the plurality of template nucleic acids, or a subset thereof.

51. The method of claim 50, wherein the forward primer is complementary to the first primer binding domain, and wherein the reverse primer is complementary to a sequence common to the plurality of template nucleic acids.

52. The method of any one of claims 49-51, wherein the method further comprises labeling the plurality of in vitro transcribed CRISPR-Cas guide RNAs according to the methods of any one of claims 35-40.

53. The method of any one of claims 49-52, wherein the method further comprises, after said transcribing of (b): contacting the plurality of in vitro transcribed CRISPR-Cas guide RNAs with a CRISPR-Cas effector protein to produce a plurality of CRISP-Cas effector complexes.

54. The method of claim 53, wherein the CRISPR-Cas effector protein is a Class 2 CRISPR-Cas effector protein.

55. The method of claim 54, wherein the Class 2 CRISPR-Cas effector protein is a Type II or Type V CRISPR-Cas effector protein.

56. A method of guiding a plurality of CRISPR-Cas effector complexes to a contiguous genomic region of interest, the method comprising contacting a contiguous genomic region in a target cell with a plurality of CRISPR-Cas effector complexes, wherein each CRISPR-Cas effector complex in the plurality of complexes comprises:
(a) a CRISPR-Cas effector protein;
(b) an in vitro transcribed guide RNA that hybridizes to a target sequence within the contiguous genomic region, thereby guiding the CRISPR-Cas effector complex to the contiguous genomic region,
wherein the plurality comprises 3 or more of said guide RNAs, and wherein the genomic region of interest is at least 500 contiguous nucleotides in length.

57. The method of claim 56, wherein the plurality comprises 3 to 20,000 of said guide RNAs, and wherein the guide RNAs to a target sequence within a genomic region of interest 500 to 2,000,000 contiguous nucleotides in length.

58. The method of claim 56 or 57, wherein the genomic region of interest is a non-repetitive genomic region.

59. The method of any one of claims 56-58, wherein the method comprises imaging, perturbing, altering, and/or regulating the genomic region of interest.

60. The method of any one of claims 56-59, wherein said guide RNAs target a target sequence selected from a list of chromatin accessible target sequences generated according to the method of any one of claims 1-7.

61. The method of any one of claims 56-60, wherein said guide RNAs are labeled according to the methods of any one of claims 35-40.

62. The method of any one of claims 56-61, wherein said CRISPR-Cas effector protein is a Class 2 CRISPR-Cas effector protein.

63. The method of claim 62, wherein the Class 2 CRISPR-Cas effector protein is Type II or Type V CRISPR-Cas effector protein.

64. The methods of any one of claims 56-63, wherein the CRISPR-Cas effector protein is catalytically inactive or has nickase activity.

65. The methods of any one of claims 56-64, wherein the CRISPR-Cas effector protein comprises a heterologous protein.

66. The method of claim 65, wherein the heterologous protein is a transcription repressor, a transcription activator, a base editor, a reverse transcriptase, or an epigenetic modifier.

67. The methods of any one of claims 56-66, wherein the target cell is a eukaryotic cell.

68. The method of claim 67, wherein the eukaryotic cell is a mammalian cell.

69. The method of claim 68, wherein the mammalian cell is a human cell.

70. The method of any one of claims 56-69, wherein the cell is in vitro.

71. The method of any one of claims 56-69, wherein the cell is ex vivo.
